(19) United States
(12) Patent Application Publication
(10) Pub. No.: US 2011/0230420 A1
(43) Pub. Date: Sep. 22, 2011

(54) RELEASABLE CONJUGATES FOR NUCLEIC ACIDS DELIVERY SYSTEMS

(75) Inventors: Hong Zhao, Edison, NJ (US); Jing Xia, Warren, NJ (US); Prasanna Reddy, Somerset, NJ (US)

(73) Assignee: ENZON PHARMACEUTICALS, INC., Bridgewater, NJ (US)

(21) Appl. No.: 13/129,705

(22) PCT Filed: Nov. 17, 2009
(86) PCT No.: PCT/US09/64711

§ 371 Date: May 24, 2011

Related U.S. Application Data
(60) Provisional application No. 61/115,350, filed on Nov. 17, 2008.
(60) Provisional application No. 61/115,326, filed on Nov. 17, 2008.

(51) Int. Cl. A61K 38/14; C12N 5/00; C07K 9/00; A61P 35/00

(52) U.S. Cl. 514/19.3; 435/375; 530/322

(57) ABSTRACT

The present invention is directed to nucleic acids delivery systems and methods of modulating an expression of a target gene using the same. In particular, the invention relates to nucleic acids conjugates containing an endosomal release-promoting moiety. The nucleic acids conjugates further contain a nuclear localization signal moiety, and/or a cell targeting moiety.

### CROSS-REFERENCE TO RELATED APPLICATION

This application claims the benefit of priority from U.S. Provisional Patent Application Ser. Nos. 61/115,350 and 61/115,326 filed Nov. 17, 2008, the contents of each of which are incorporated herein by reference.

### BACKGROUND OF THE INVENTION

Targeted delivery is a promising approach to improve the efficacy of therapeutic molecules. Over the years, numerous methods have been proposed for selectively delivering therapeutic molecules, such as oligonucleotides, into the body and improving bioavailability of these medicinal agents. However, there have been obstacles for clinicians to use nucleic acids because nucleic acids such as oligonucleotides have a highly negatively charged backbone which hinders nucleic acids from crossing cellular membranes.

It is desirable to provide a targeted delivery system which enhances cellular uptake and increases bioavailability of oligonucleotides in cells, i.e., cancer cells. In spite of the attempts and advances, there continues to be a need to provide an improved targeted delivery system. The present invention addresses this need.

### SUMMARY OF HE INVENTION

In order to overcome the above problems and improve the technology for the delivery of oligonucleotides, there are provided ad oleic acids conjugates containing an acid labile linker.

In one aspect of the present invention, there are provided compounds of Formula (I):

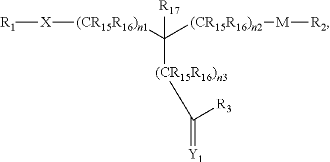

wherein

R₁ is a group of Formula (Ia₁) or (Ia₂):

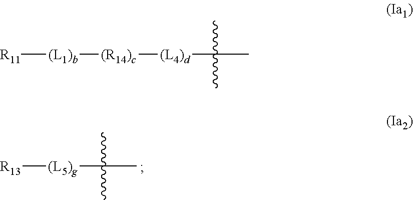

X is O or S;

R₂ is hydrogen, a leaving group, a functional group, a targeting group, a non-antigenic polymer, or a group of Formula (Ib₁), (Ib₂), or (Ib₃):

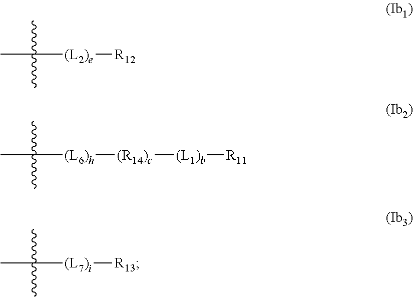

M is O, or NR₅;
R₃ is OH, OR₆, SH, SR₇, a leaving group, a functional group, a targeting group, a non-antigenic polymer or a group of Formula (Ic₁) or (Ic₂) or (Ic₃);

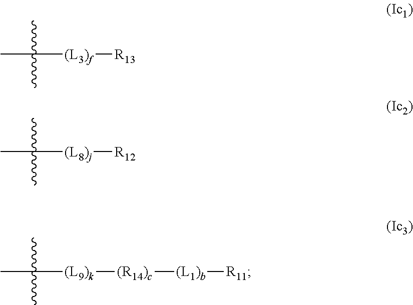

Y₁ is O, S, or NR₈;

R₄ alkyl, C₁₋₆ alkyl, C₁₋₆ branched alkyl or

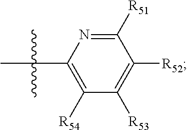

wherein R₅₁₋₅₄ are independently selected from among hydrogen, amino, azido, carboxy, cyano, halo, hydroxyl, nitro, hydrogen, C₁₋₆ alkyl, C₃₋₈ branched alkyl, C₃₋₈ cycloalkyl, C₁₋₆ substituted alkyl, C₃₋₈ substituted cycloalkyl, aryl and substituted aryl;

R₅ and R₈ are independently selected from among hydrogen, amino, azido, carboxy, cyano, halo, hydroxyl, nitro, C₁₋₆ alkyl, C₃₋₈ branched alkyl, C₃₋₈ cycloalkyl, C₁₋₆ substituted alkyl, C₃₋₈ substituted cycloalkyl, aryl and substituted aryl;

R₆ and R₇ are independently C₁₋₆ alkyl, or C₁₋₆ branched alkyl;

R₁₁ is hydrogen, C₁₋₆ alkyl, a functional group, a targeting group, or an endosomal release-promoting moiety;

R₁₂ is hydrogen, C₁₋₆ alkyl, a leaving group, a functional group, a targeting group, a nuclear localization signal peptide, or a non-antigenic polymer;

R₁₃ is selected from among OH, OR₆, SH, SR₇, a leaving group, a functional group, a targeting group, a biologically active agent, and a non-antigenic polymer, or

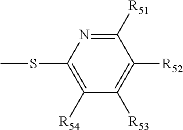

wherein a group of Formula (Ia₂) is present and (g) is zero;

R₁₄ is an endosomal release-promoting moiety;

R₁₅₋₁₇ are independently selected, from among hydrogen, hydroxyl, C₁₋₆ alkyls, C₂₋₆ alkenyl, C₂₋₆ alkynyl, C₃₋₁₉ branched alkyl, C₃₋₈ cycloalkyl, and C₁₋₆ alkoxy, wherein R₁₅₋₁₇ in each occurrence are independently the same or different;

L₁₋₃ and L₆₋₉ are independently selected bifunctional linkers, wherein L₁₋₃ and L₆₋₉ in each occurrence are independently the same or different;

L₄₋₅ are independently selected bifunctional spacers containing a terminal sulfur adjacent to X;

(c) is zero or 1;

(d) and (g) are independently zero or 1;

(b), (e), (f), (h), (i), (j) and (k) are independently zero or positive integers;

(n1) is zero or a positive integer of from about 1 to about 10;

(n2) and (n3) are independently zero or positive integers of from about 1 to about 10, provided that at least one of R₁₋₃ includes an endosomal release-promoting moiety, and provided that at least one of the remaining R₁₋₃ includes a biologically active agent, or

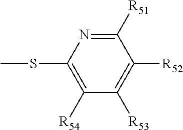

wherein a group of Formula (Ia₂) is present and (g) is zero.

In another aspect of the invention, there are provided methods of preparing the compounds described herein.

In yet another aspect of the invention, there are provided methods of inhibiting gene expression in a mammal for the treatment of various diseases e.g., cancer. Preferably, the targeted gene includes oncogenes, pro-angiogenesis pathway genes, pro-cell proliferation pathway genes, viral infectious agent genes, and pro-inflammatory pathway genes.

One advantage of the present invention is that the nucleic acids transport systems provide a means for intracellular delivery of therapeutic agents such as oligonucleotides. The present invention facilitates cellular uptake of oligonucleotides and allows selective regulation of target gene expression. This selective regulation technology allows enhanced efficacy of therapeutic agents and decrease in toxicity.

Another advantage is that the present invention allows targeted delivery of therapeutic agents. For example, folate receptor is highly expressed in many cancer cells and tissues. Folic acid is bound to folate receptors expressed on the cancer cell membranes, and enters the cells through a process called a receptor mediated endocytosis. Useful therapeutic agent conjugates attached to folate can be internalized into the cells via the folate-targeted process, folate receptor mediated endocytosis

Yet another advantage is that the present invention enhances endosomal release of therapeutic agents to the cytoplasm. Without being bound by any theory, the endosomal release-promoting groups such as histidine-rich peptides can destabilize the endosomal membranes, thereby facilitating cytoplasmic delivery of therapeutic agents. Histidine-rich peptides can undergo a shift in their properties (e.g., a shift in; hydrophobicity or ability to interact with endosomal membranes) in acidic environment by proton sponge effect, thereby disrupting and/or destabilizing endosome and promoting release of endosomal contents into the cytoplasm. Then, the intracellularly released therapeutic agents can translocate to the nucleus.

Yet another advantage is that the nucleic acids transport systems contain an acid labile linker which facilitate release of therapeutic agents and escape from endosomal compartments to cytoplasm.

Oligonucleotides attached to the compounds described herein can enter targeted area, such as cancer cells, thus allowing the artisan to achieve a desired bioavailability of therapeutic oligonucleotides at a targeted area. In addition, release of the oligonucleotides can be modified in different cellular compartments. Thus, the nucleic acids transport systems described, herein allow sufficient amounts of the therapeutic oligonucleotides to be selectively available at the desired target area, i.e. the cytoplasm and the nucleus.

A further advantage of the present invention is that the conjugates described herein allow cellular uptake and specific mRNA down regulation in cancer cells in the absence of transfection agents. This is a significant advantage over prior art technologies, and thus significantly simplifies treatment regimens, i.e. the in vivo administration of oligonucleotide drugs. This technology can be applied to the in vivo administration of therapeutic oligonucleotides including LNA oligomers.

For purposes of the present invention, the term “residue” shall be understood to mean that portion of a compound, to which it refers, i.e. endosomal release-promoting group, PEG, oligonucleotide, etc. that remains after it has undergone a substitution reaction with another compound.

For purposes of the present invention, the term “polymeric residue” or “PEG residue” shall each be understood to mean that portion of the polymer or PEG which remains after it has undergone a reaction with other compounds, moieties, etc.

For purposes of the present invention, the term “alkyl” as used herein refers to a saturated aliphatic hydrocarbon, including straight-chain, branched-chain, and cyclic alkyl groups. The term “alkyl” also includes alkyl-thio-alkyl, alkoxyalkyl, cycloalkylalkyl, heterocycloalkyl, C₁₋₆ hydrocarbonyl, groups. Preferably, the alkyl group has 1 to 12 carbons. More preferably, it is a lower alkyl of from about 1 to 7 carbons, yet more preferably about 1 to 4 carbons. The alkyl group can be substituted or unsubstituted. When substituted, the substituted group(s) preferably include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

For purposes of the present invention, the term “substituted” as used herein refers to adding or replacing one or more atoms contained within a functional group or compound with one of the moieties from the group of halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

The term “alkenyl” as used herein refers to groups containing, at least one carbon-carbon double bond, including straight-chain, branched-chain, and cyclic groups. Preferably, the alkenyl group has about 2 to 12 carbons. More preferably, it is a lower alkenyl of from about 2 to 7 carbons, yet more preferably about 2 to 4 carbons. The alkenyl group on be substituted or unsubstituted. When substituted, the substituted group(s) preferably include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

The term “alkynyl” as used herein, refers to groups containing at least one carbon-carbon triple bond, including straight-chain, branched-chain, and cyclic groups. Preferably, the alkynyl group has about 2 to 12 carbons. More preferably, it is a lower alkynyl of from about 2 to 7 carbons, yet more preferably about 2 to 4 carbons. The alkynyl group can be substituted or unsubstituted. When substituted, the substituted group(s) preferably include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups. Examples of “alkynyl” include propargyl, propyne, and 3-hexyne.

The term “aryl” as used herein refers to an aromatic hydrocarbon ring system containing at least one aromatic ring. The aromatic ring can optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl.

The term “cycloalkyl” as used herein refers to a C₃₋₈ cyclic hydrocarbon. Examples of cycloalkyl include cyclopropyl, cyclobutyl, cyclopentyl, cyclohexyl, cycloheptyl and cyclooctyl.

The term “cycloalkenyl” as used herein refers to a C₃₋₈ cyclic hydrocarbon containing at least one carbon-carbon double bond. Examples of cycloalkenyl include cyclopentenyl, cyclopentadienyl, cyclohexenyl, 1,3-cyclohexadienyl, cycloheptenyl, cycloheptatrienyl, and cyclooctenyl.

The term “cycloalkylalkyl” as used herein refers to an alklyl group substituted with a C₃₋₈ cycloalkyl group. Examples of cycloalkylalkyl groups include cyclopropylmethyl and cyclopentylethyl.

The term “alkoxy” as used herein refers to an alkyl group of indicated number of carbon atoms attached to the parent molecular moiety through an oxygen bridge. Examples of alkoxy groups include, for example, methoxy, ethoxy, propoxy and isopropoxy.

An “alkylaryl” group as used herein refers to an aryl group substituted with an alkyl group.

An “aralkyl” group as used herein refers to an alkyl group substituted with an aryl group.

The term “alkoxyalkyl” group as used herein refers to an alkyl group substituted with an alkloxy group.

The term “alkyl-thio-alkyl” as used herein refers to an alkyl-S-alkyl thioether, for example, methylthiomethyl or methylthioethyl.

The term “amino” as used herein refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups, respectively.

The term “alkylcarbonyl” as used herein refers to a carbonyl group substituted with alkyl group.

The terms “halogen” or “halo” as used herein refer to fluorine, chlorine, bromine, and iodine.

The term “heterocycloalkyl” as used herein refers to a non-aromatic ring system containing at least one heteroatom selected from nitrogen, oxygen, and sulfur. The heterocycloalkyl ring can be optionally fused to or otherwise attached to other heterocycloalkyl rings and/or non-aromatic hydrocarbon rings. Preferred heterocycloalkyl groups have from 3 to 7 members. Examples of heterocycloalkyl groups include, for example, piperazine, morpholine, piperidine, tetrahydrofuran, pyrrolidine, and pyrazole. Preferred heterocycloalkyl groups include piperazinyl, piperazinyl, morpholinyl, and pyrrolidinyl.

The term “heteroaryl” as used herein refers to an aromatic ring system containing at least one heteroatom selected from nitrogen, oxygen, and sulfur. The heteroaryl ring can be fused or otherwise attached to one or more heteroaryl rings, aromatic or non-aromatic hydrocarbon rings or heterocycloalkyl rings. Examples of heteroaryl groups include, for example, pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl.

The term “heteroatom” as used herein refers to nitrogen, oxygen, and sulfur.

In some embodiments, substituted alkyls include carboxyalkyls, aminoalkyls, dialkylaminos, hydroxyalkyls and mercaptoalkyls; substituted alkenyls include carboxyalkenyls, aminoalkenyls, dialkenylaminos, hydroxyalkenyls and mercaptoalkenyls; substituted alkynyls include carboxyalkynyls, aminoalkynyls, dialkynylaminos, hydroxyalkynyls and mercaptoalkynyls; substituted cycloalkyls include moieties such as 4-chlorocyclohexyl; aryls include moieties such as napthyl; substituted aryls include moieties such as 3-bromo phenyl; aralkyls include moieties such as tolyl; heteroalkyls include moieties such as ethylthiophene; substituted heteroaryls include moieties such as 3-methoxythiophene; alkoxy includes moieties such as methoxy; and phenoxy includes moieties such as 3-nitrophenoxy. Halo shall be understood to include fluoro, chloro, iodo and bromo.

For purposes of the present invention. “positive integer” shall be understood to include an integer equal to or greater than 1 and as will be understood by those of ordinary skill to be within the realm of reasonableness by the artisan of ordinary skill, preferably from 1 to about 10, more preferably 1 or 2 in some embodiments.

For purposes of the present invention, the term “linked” shall be understood to include covalent (preferably) or noncovalent attachment of one group to another, i.e., as a result of a chemical reaction.

The terms “effective amounts” and “sufficient amounts” for purposes of the present invention shall mean an amount which achieves a desired effect or therapeutic effect as such effect is understood by those of ordinary skill in the art.

For purposes of the present invention, the term “therapeutic oligonucleotide” refers to an oligonucleotide used as a pharmaceutical or diagnostic agent.

For purposes of the present invention, “modulation of genie expression” shall be understood as broadly including down-regulation or up-regulation of any types of genes, preferably associated with cancer and inflammation, compared to a gene expression observed in the absence of the treatment with the compounds described herein, regardless of the route of administration.

For purposes of the present invention, “inhibition of gene expression” of a target gene shall be understood to mean that mRNA expression or protein translated are reduced or attenuated when compared to that observed in the absence of the treatment with the compound described herein. Suitable assays include, e.g., examination of protein or mRNA levels using techniques known to those of skill in the art such as dot blots, northern blots, in situ hybridization, ELISA, immunoprecipitation, enzyme function, as well as phenotypic assays known to those of skill in the art. The treated conditions can be confirmed by, for example, decrease in mRNA levels in cells, preferably cancer cells or tissues.

Broadly speaking, successful inhibition or treatment shall be deemed to occur when the desired response is obtained. For example, successful inhibition or treatment earl be defined by obtaining e.g., 10% or higher (i.e. 20% 30%, 40%) down regulation of genes associated with tumor growth inhibition. Alternatively, successful treatment can be defined by obtaining at least 20% or preferably 30%, more preferably 40% or higher (i.e., 50% or 80%) decrease in oncogene mRNA levels in cancer cells or tissues, including other clinical markers contemplated by the artisan in the field, when compared to that observed in the absence of the treatment with the compound described herein.

Further, the use of singular terms for convenience in description is in no way intended to be so limiting. Thus, for example, reference to an oligonucleotide, a compound of Formula (I), a cationic lipid, a fusogenic lipid, a PEG lipid etc., refers to one or more molecules of that oligonucleotide, compound of Formula (I), cationic lipid, fuosogenic lipid, PEG lipid, etc. It is also contemplated that the oligonucleotide can be the same or different kind of gene. It is also to be understood that this invention is not limited to the particular configurations process steps, and materials disclosed herein as such configurations, process steps, and materials may vary somewhat.

It is also to be understood that the terminology employed herein is used for the purpose of describing particular embodiments only and is not intended to be limiting, since the scope of the present invention will be limited by the appended claims and equivalents thereof.

### BRIEF DESCRIPTION OF THE DRAWINGS

FIG. 1 shows a schematic diagram of components of compound of Formula (I).

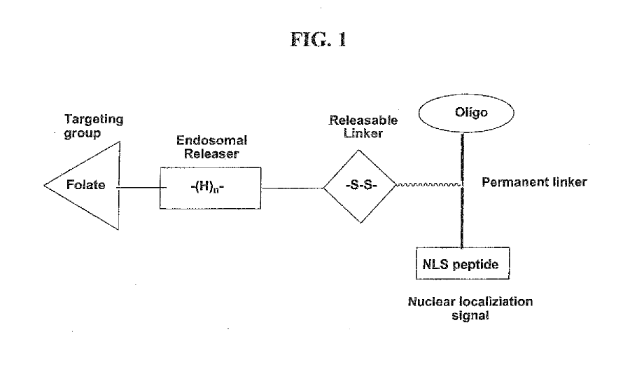

FIG. 2 schematically illustrates a reaction scheme of compounds 5 and 5a, as described in Examples 6-10.

FIG. 3 schematically illustrates a reaction scheme of compounds 16 and 16a, as described in Examples 11-23.

FIG. 4 is an image of cells treated with oligonucleotides labelled with FAM, shown fluorescing, and illustrating cellular uptake and cytoplasmic localization of oligonucleotides, as described in Example 24.

### DETAILED DESCRIPTION OF THE INVENTION

### A. Compounds of Formula (I)

### 1. Overview

It one aspect of the present invention, there are provided compounds of Formula (I):

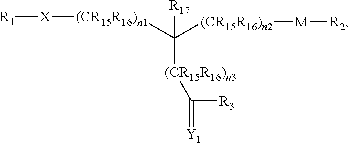

wherein

R₁ is a group of Formula (Ia₁) or (Ia₂):

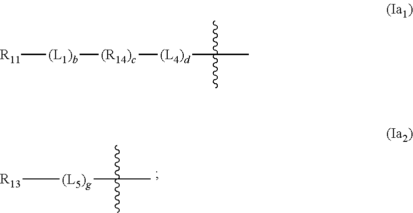

X is O or S, preferably S;

R₂ is hydrogen, a leaving group, a functional group, a targeting group, a non-antigenic polymer, or a group of Formula (Ib₁), (Ib₂), or (Ib₃):

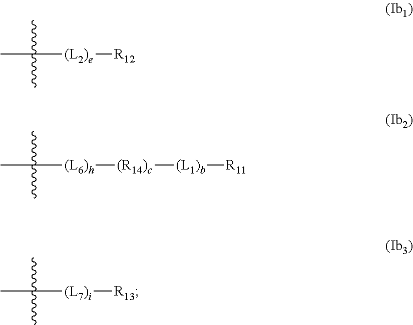

M is O, or NR₅, preferably NR₅:

R₃ is OH, OR₆, SH, SR₇, a leaving group, a functional group, a targeting group, a non-antigenic polymer or a group of Formula (Ic₁), (Ic₂) or (Ic₃);

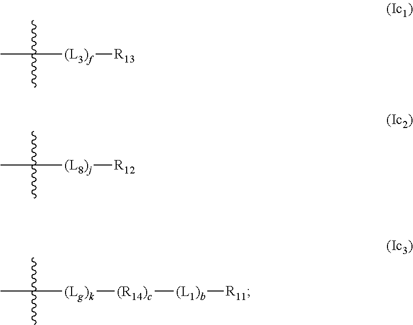

Y₁ is O, S, or NR₈, preferably O;

R₄ is C₁₋₆ alkyl, C₁₋₆ branched alkyl or

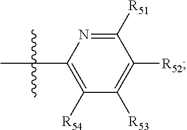

wherein R₅₁₋₅₄ are independently selected from among hydrogen, amino, azido, carboxy, cyano, halo, hydroxyl, nitro, C₁₋₆ alkyl, C₃₋₈ branched alkyl, C₃₋₈ cycloalkyl, C₁₋₆ substituted alkyl, C₃₋₈ substituted cycloalkyl, aryl and substituted aryl, preferably R₅₁ is nitro and R₅₂₋₅₄ are hydrogen;

R₅ and R₈ are independently selected from among hydrogen, amino, azido, carboxy, cyano, halo, hydroxyl, nitro, C₁₋₆ alkyl, C₃₋₈ branched alkyl, C₃₋₈ cycloalkyl, C₁₋₆ substituted alkyl, C₃₋₈ substituted cycloalkyl, aryl and substituted aryl, preferably, hydrogen, methyl, ethyl and propyl;

R₆ and R₇ are independently C₁₋₆ alkyl (e.g., methyl, ethyl, propyl) or C₃₋₈ branched alkyl (tertiary butyl);

R₁₁ is hydrogen, C₁₋₆ alkyl (e.g., methyl, ethyl, propyl), a functional group, a targeting group, or an endosomal release-promoting moiety;

R₁₂ is hydrogen, C₁₋₆ alkyl (e.g., methyl, ethyl, propyl), a leaving group, a functional group, a targeting group, a nuclear localization signal peptide, or a non-antigenic polymer;

R₁₃ is selected from among OH, OR₆, SH, SR₇, a leaving group, a functional group, a targeting group, a biologically active agent, and a non-antigenic polymer, or

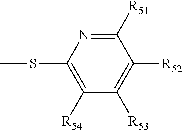

wherein a group of Formula (Ia₂) is present and (g) is zero;

R₁₄ is an endosomal release-promoting moiety;

R₁₅₋₁₇ are independently selected from among hydrogen, hydroxyl, C₁₋₆ alkyls, C₂₋₆ alkenyl, C₂₋₆ alkynyl, C₃₋₁₉ branched alkyl, C₃₋₈ cycloalkyl, and C₁₋₆ alkoxy, wherein R₁₅₋₁₇ in each occurrence are independently the same or different when (n1), (n2) or (n3) is equal to or greater than 2;

L₁₋₃ and L₆₋₉ are independently selected bifunctional linkers, wherein L₁₋₃ and L₆₋₉ in each occurrence are independently the same or different when (b), (e), (f), (h), (i), (j) or (k) is equal to or greater than 2;

L₄₋₅ are independently selected bifunctional spacers containing a terminal sulfur adjacent to X;

(c) is zero or 1;

(d) and (g) are independently zero or 1, preferably 1;

(b), (e), (f), (h), (i), (j) and (k) are independently zero or positive integers 1, 2, 3, 4, 5, 6);

(n1) is zero or a positive integer of from about 1 to about 10, preferably 0, 1, 2, 3, 4, 5, 6, more preferably 0, 1, 2, 3, and yet more preferably 1;

(n2) and (n3) are independently zero or positive integers of from about 1 to about 10, preferably 0, 1, 2, 3, 4, 5, 6, more preferably, 0, 1, 2, 3, and yet more preferably 1, provided that at least one of R₁₋₃ (i.e., R₁) includes an endosomal release-promoting moiety, and provided that at least one of the remaining R₁₋₃ (e.g., R₁ or R₃) includes a biologically active agent, or

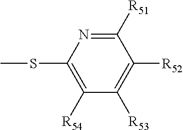

wherein a group of Formula (Ia₂) is present and (g) is zero.

In one preferred aspect, the present invention provides compounds of Formula (I) in which one of R₁₋₃ includes an endosomal release-promoting moiety, and at least one of the remaining R₁₋₃ includes a biologically active agent.

In another preferred aspect, the present invention provides compounds in which R₁ includes an endosomal release-promoting moiety, and one of the remaining R₂₋₃ includes a biologically active agent; or

R₁ includes a biologically active agent or

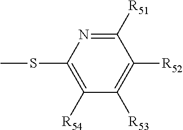

wherein (g) is zero, and one of the remaining R₂₋₃ includes, an endosomal release-promoting moiety.

Preferably, R₁ includes an endosomal release-promoting moiety, and one of the remaining R₂₋₃ includes a biologically active agent; or R₁ includes a biologically active agent and one of the remaining R₂₋₃ includes an endosomal release-promoting moiety. The present invention provides compounds in which an endosomal release-promoting group or a biologically active agent is releasably linked to the core structure of the compounds.

In certain embodiments, the present invention provides compound of Formula (I) wherein:

R₁ is a group of Formula (Ia₁) or (Ia₂):

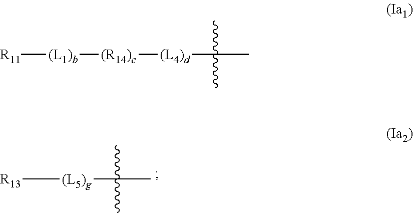

R₂ is a group of Formula (Ib₁), (Ib₂), or (Ia₃):

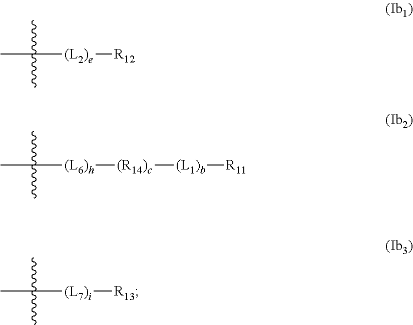

and

R₃ is OH, OR₆, or a group of Formula (Ic₁), (Ic₂) or (Ic₃):

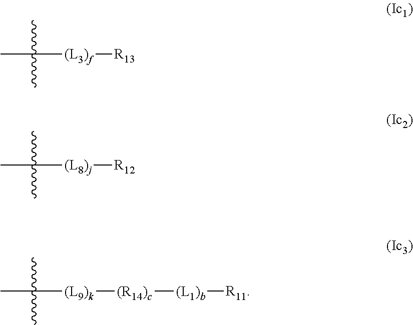

Preferably, at least one of R₁₁ and R₁₄ includes an endosomal release-promoting moiety, R₁₂ is a nuclear localization signal peptide; and R₁₃ includes a biologically active agent.

In certain embodiments, the compounds described herein have Formula (IIa) or (II′a):

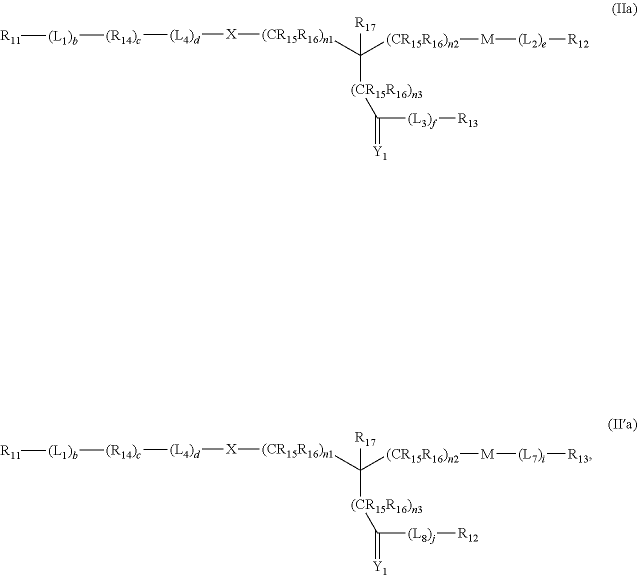

wherein at lest one of R₁₁ and R₁₄ includes an endosomal release-promoting moiety and R₁₃ includes a biologically active agent.

In certain embodiments, the compounds described herein have Formula (IIb) or (II′b):

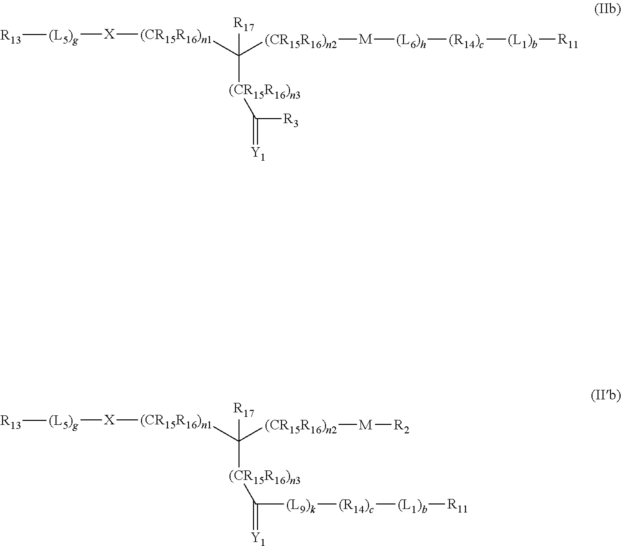

wherein

at least one of R₁₁ and R₁₄ includes an endosomal release-promoting moiety;

R₁₃ is biologically active agent when (g) is zero or 1, or

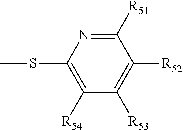

when (g) is zero;

R₂ is hydrogen, a leaving group, a functional group, a targeting group, a non-antigenic polymer; and

R₃ is OH, OR₆, SH, SR₇, a leaving group, a functional group, a targeting group, a non-antigenic polymer.

In one preferred embodiment, R₁₃ is a biologically active agent and (g) is zero.

In another, aspect of the present invention, the biologically active agent is selected from among —NH₂ containing moieties, —OH containing moieties and —SH containing moieties. Alternatively, the biologically active agents include, but are not limited to, pharmaceutically active compounds/agents and nucleic acids such as oligonucleotides.

In certain embodiments, the biologically active agent is a biologically active agent containing neutral or negative charges. Such negatively charged compounds include, but are not limited to, pharmaceutically active compounds, and nucleic acids such as an oligonucleotide.

For purposes of the present invention, pharmaceutically active compounds shall be mean to include small molecules such as those having an average molecular weight of less than about 1,500 daltons).

For ease of description and not limitation, it will be understood that the term “small molecules” are interchangeable with “pharmaceutically active compounds”.

In one preferred aspect, the biologically active agent includes an oligonucleotide.

In another aspect of the invention, R₁ is a biologically active agent releasably linked to X via a disulfide bond. In yet another aspect, R₁ includes an endosomal release-promoting moiety releasably linked to X via a disulfide bond.

In yet another preferred aspect of the invention, the compounds of Formula (I) contain an endosomal release-promoting group or a combination plan endosomal release-promoting group and a targeting group, and a biologically active agent.

In one embodiment, R₁ includes an endosomal release-promoting group or a combination of an endosomal release-promoting group and a targeting group; and R₃ includes a biologically active agent.

In another embodiment, R₁ includes an endosomal release-promoting group or combination of an endosomal release-promoting group and a targeting group; and R₂ includes a biologically active agent.

In yet another embodiment, R₁ includes a biologically active agent and R₂ includes an endosomal release-promoting group or a combination of an endosomal release-promoting group and a targeting group.

In yet another embodiment, R₁ includes a biologically active agent, and R₃ includes an endosomal release-promoting group or a combination of an endosomal release-promoting group and a targeting group.

In certain embodiments, R₁ includes an endosomal release-promoting group or a combination of an endosomal release-promoting group and a targeting group; R₂ includes a biologically active agent; and R₂ includes a nuclear localization signal group.

In certain embodiments, R₁ an endosomal release-promoting group or a combination of an endosomal release-promoting group and a targeting group; R₂ includes a biologically active agent; and R₃ includes a nuclear localization signal group.

In certain embodiments, R₁ includes a biologically active agent and R₂ includes an endosomal release-promoting group or a combination of an endosomal release-promoting group and a targeting group, and R₃ is OH.

In certain embodiments, R₁ includes a biologically active agent and R₃ includes an endosomal release-promoting group or a combination of an endosomal release-promoting group and a targeting group, and R₂ is hydrogen.

Preferably, X is S; Y₁ is O; and M is NH.

In a further aspect, compounds of Formula (I) containing a water-soluble and non-antigenic polymer are contemplated. For example, a non-antigenic polymer such as polyalkylene oxide is conjugated to an endosomal release-promoting group or a targeting group. A targeting group-modified polyalkylene oxide is also contemplated. Alternatively, a biologically active agent conjugated to a non-antigenic polymer is also contemplated.

One preferred aspect of the invention is that (n1) is 1, and both (n2) and (n3) are zero. The compounds described herein have Formula (III):

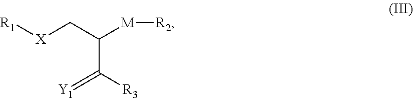

In certain embodiments, R₁, R₂ and R₃ have Formulae (Ia₁), (Ib₁) and (Ic₁):

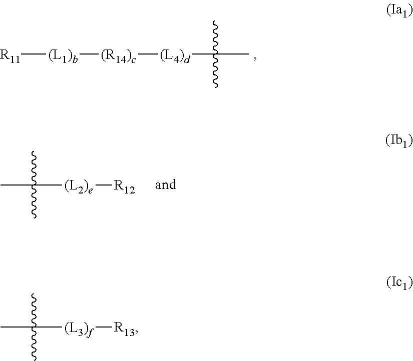

respectively.

In certain embodiments, R₁, R₂ and R₃ have Formulae (Ia₁), (Ib₃) and (Ic₂):

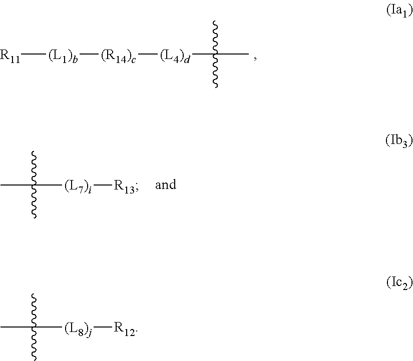

In certain embodiments, R₁₁ is a targeting group (e.g., a cell surface targeting moiety), R₁₄ is an endosomal release-promoting moiety, and (c) is 1.

In certain embodiments, R₁₁ is an endosomal release-promoting moiety, and (c) is zero.

In certain embodiments, (b) is zero or an positive integer (i.e., 0, 1, 2).

Alternatively, the compounds described herein have Formula (IIIa) or (III′a):

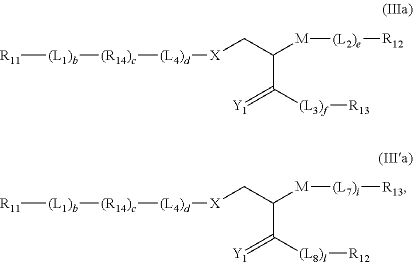

wherein at least one of R₁₁ and R₁₄ includes an endosomal release-promoting moiety, and R₁₃ includes a biologically active agent.

In certain embodiments, at least one of R₁₁ and R₁₄ includes an endosomal release-promoting moiety, R₁₃ includes a biologically active agent, and R₁₂ is a nuclear localization signal peptide.

In one embodiment, R₁₁ is a targeting group (e.g., a cell surface targeting moiety); R₁₄ is an endosomal release-promoting moiety, and (c) is 1; R₁₃ includes a biologically active agent; and R₁₂ is a nuclear localization signal peptide.

In another embodiment, R₁₁ is an endosomal release-promoting moiety, and (c) is zero; R₁₃ includes a biologically active agent; and R₁₂ is a nuclear localization signal peptide.

In certain embodiments, R₁ and R₂ have Formulae (Ia₂), and (Ib₂);

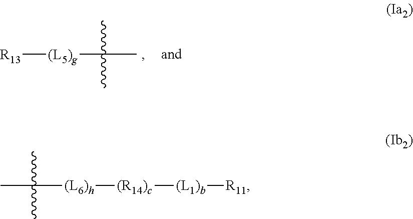

respectively.

In certain embodiments; R₁ and R₃ have Formulae (Ia₂), and (Ic₃):

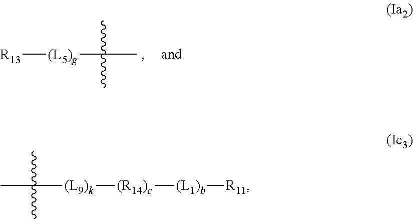

respectively.

Alternatively, the compounds described herein Formula (IIIb) or (III′b):

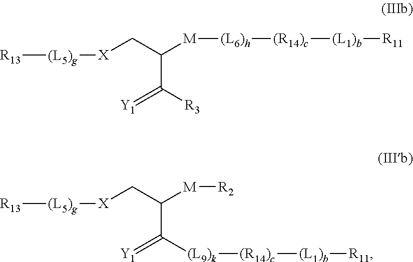

wherein

at least one of R₁₁ and R₁₄ includes an endosomal release-promoting moiety;

R₁₃ is a biologically active agent when (g) is zero or 1, or

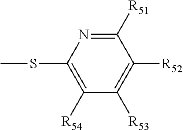

wherein (g) is zero;

R₂ is hydrogen, a leaving group, a functional group, a targeting group, a non-antigenic polymer; and

R₃ is OH, OR₆, a leaving group, a functional group, a targeting group, a non-antigenic polymer.

In certain embodiments, at least one of R₁₁ and R₁₄ includes an endosomal release-promoting moiety, and R₁₃ includes a biologically active agent.

In one embodiment, R₁₁ is a targeting group (e.g., a cell surface targeting moiety); R₁₄ is an endosomal release-promoting moiety, and (c) is 1; and R₁₃ includes a biologically active agent.

In another embodiment, R₁₁ is an endosomal release-promoting moiety, and (c) is zero; R₁₃ includes a biologically active agent.

In certain preferred embodiments, the compounds described herein have Formula (IVa) or (IV′a):

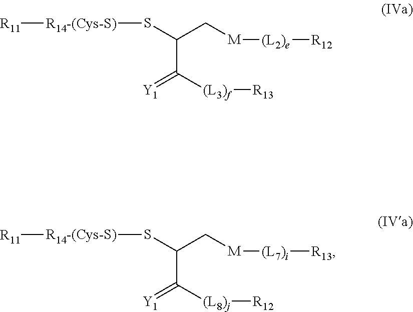

wherein

R₁₁ is hydrogen, a targeting group or a histidine-rich peptide;

R₁₂ is hydrogen, C₁₋₆ alkyl, a leaving group, a functional group, a nuclear localization signal peptide or a non-antigenic polymer;

R₁₃ is a biologically active agent; and

R₁₄ includes a histidine-rich peptide.

In certain embodiments, the compounds described herein have Formula (IVb) or (IV′b):

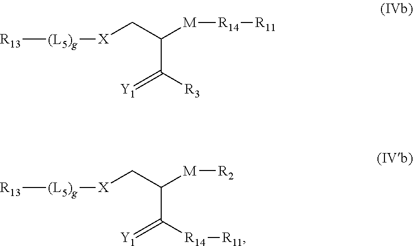

wherein

R₁₁ is hydrogen, a targeting group or a histidine-rich peptide;

R₁₃ is a biologically active agent when (g) is zero or 1, or

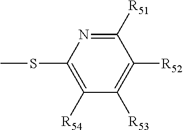

wherein (g) is zero;

R₁₄ includes a histidine-rich peptide;

R₂ is hydrogen, a leaving group, a functional group, a targeting group, a non-antigenic polymer; and

R₃ is OH, OR₆, a leaving group, a functional group, a targeting group, a non-antigenic polymer.

Preferably, R₁₃ is a biologically active agent.

In one preferred embodiment, R₁ includes a biologically active agent releasably linked to X (sulfur).

The histidine-rich peptide contains about 3 to about 40 amino acids, preferably about 3 to about 25 amino acids (e.g., 3, 4, 5, 6, 7, 8, 9, 10, 11, 12, 13, 14, 16, 16, 17, 18, 19, 20, 21, 22, 23, 24, 25).

In one preferred embodiment, the endosomal release-promoting moiety includes (His)ₙ, wherein His is a histidine, and (n) is a positive integer, preferably a positive integer equal to or greater than 3, (e.g., a positive integer of from about 3 to about 20). For example, the endosomal release-promoting moiety includes -His-His-His-.

In one embodiment, there are provided compounds of Formula (Va) or (V′a):

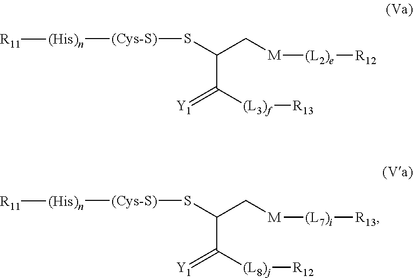

wherein

R₁₁ is hydrogen or a targeting group;

R₁₂ is hydrogen, C₁₋₆ alkyl, a leaving group, a functional group, or a nuclear localization signal peptide;

R₁₃ includes a biologically active agent;

His is histidine; and

(n) is a positive integer equal to or greater than 3 (e.g., 3, 4, 5, 6, 7, 8, 9, 10, 11, 12, 13, 14, 15, 16).

In certain embodiments, the (His)ₙ moiety optionally includes lysine.

In another embodiment, the compounds described herein have Formula (Vb) or (V′b);

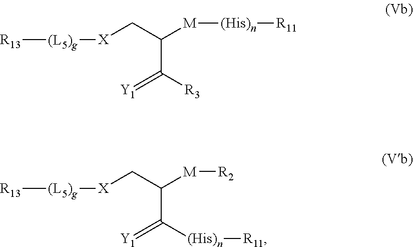

wherein

R₁₁ is hydrogen or a targeting group;

R₁₃ is a biologically active agent when (g) is zero or 1, or

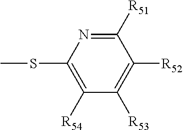

wherein (g) is zero;

R₂ is hydrogen, a leaving group, a functional group, a targeting group, a non-antigenic polymer;

R₃ is OH, OR₆, a leaving group, a functional group, a targeting group, a non-antigenic polymer;

His is histidine; and

(n) is a positive integer equal to or greater than 3 (e.g., 3, 4, 5, 6, 7, 8, 9, 10, 11, 12, 13, 14, 15, 16).

Preferably, R₁₃ is a biologically active agent.

In one embodiment, R₁ includes a histidine-rich peptide, R₂ is permanently linked to M, and R₃ is permanently linked to C(═Y₁).

The compounds described herein include a nuclear localization signal peptide, for example, but not limited to, CGVKRKKKP (SEQ ID NO: 28), CYGRKKRRQRRR (SEQ ID NO: 29), YGRKKRRQRRRC (SEQ ID NO: 30) and YGRKKRRQRRR (SEQ ID NO: 31).

In one preferred embodiment, (c) is 1; R₁₄ is a histidine-rich peptide; and R₁₁ is a cell surface-targeting group. Preferably, the cell surface targeting group is folate or anisamide.

In another preferred embodiment, (b) and (c) are both zero, (d) is one, and R₁₁ is a histine-rich peptide.

In a further embodiment, R₁₁ includes a non-antigenic polymer such as a targeting group modified with polyalkylene oxide (e.g., a targeting group modified with polyalkylene oxide at the distal terminal of the targeting group). Alternatively, R₁₃ includes a non-antigenic polymer such as a biologically active agent modified with polyalkylene oxide (e.g. an oligonucleotide modified with polyalkylene oxide at the distal terminal of the oligonucleotide).

### 2. Linkers: L₁₋₃ and L₆₋₉ Groups

L₁₋₃ and L₆₋₉, as included compounds of Formula (I), are independently selected from among:

—(CR₂₁R₂₂)ₜ₁—[C(═Y₁₆)]ₐ₃—,

—(CR₂₁R₂₂)ₜ₁Y₁₇—(CR₂₃R₂₄)ₜ₂—(Y₁₈)ₐ₂—[C(═Y₁₆)]ₐ₃—,

—(CR₂₁R₂₂CR₂₃R₂₄Y₁₉)ₜ₁—[C(═Y₁₆)]ₐ₃—,

—(CR₂₁R₂₂CR₂₃R₂₄Y₁₇)ₜ₁(CR₂₅R₂₆)ₜ₄—(Y₁₈)ₐ₂—[C(═Y₁₆)]ₐ₃—,

—[(CR₂₁R₂₂CR₂₃R₂₄)ₜ₂Y₁₇]ₜ₃(CR₂₅R₂₆)ₜ₄—(Y₁₈)ₐ₂—[C(═Y₁₆)]ₐ₃—,

—(CR₂₁R₂₂)ₜ₁—[(CR₂₃R₂₄)ₜ₂Y₁₇]ₜ₃(CR₂₅R₂₆)ₜ₄—(Y₁₈)ₐ₂—[C(═Y₁₆)]ₐ₃—,

—(CR₂₁R₂₂)ₜ₁(Y₁₇)ₐ₂[C(═Y₁₆)]ₐ₃(CR₂₃R₂₄)ₜ₂—,

—(CR₂₁R₂₂)ₜ₁(Y₁₇)ₐ₂[C(═Y₁₆)]ₐ₃Y₁₄(CR₂₃R₂₄)ₜ₂—,

—(CR₂₁R₂₂)ₜ₁(Y₁₇)ₐ₂[C(═Y₁₆)]ₐ₃(CR₂₃R₂₄)ₜ₂—Y₁₅—(CR₂₃R₂₄)ₜ₃—,

—(CR₂₁R₂₂)ₜ₁(Y₁₇)ₐ₂[C(Y₁₆)]ₐ₃Y₁₄(CR₂₃R₂₄)ₜ₂—Y₁₅—(CR₂₃R₂₄)ₜ₃—,

—(CR₂₁R₂₂)ₜ₁(Y₁₇)ₐ₂[C(═Y₁₆)]ₐ₃(CR₂₃R₂₄CR₂₅R₂₆Y₁₉)ₜ₂(CR₂₇CR₂₈)ₜ₃—,

—(CR₂₁R₂₂)ₜ₁(Y₁₇)ₐ₂[C(═Y₁₆)]ₐ₃Y₁₄(CR₂₃R₂₄CR₂₅R₂₆Y₁₉)ₜ₂(CR₂₇CR₂₈)ₜ₃—, and

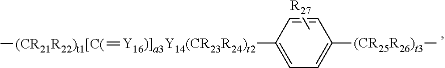

wherein:

Y₁₆ is O, NR₂₈, or S, preferably O;

Y₁₄₋₁₅ and Y₁₇₋₁₉ are independently O, NR₂₉, or S, preferably O or NR₂₉;

R₂₁₋₂₇ are independently selected from among hydrogen, hydroxyl, carboxyl, amine, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy, preferably hydrogen, methyl, ethyl and propyl;

R₂₈₋₂₉ are independently selected from throng hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy, preferably hydrogen, methyl, ethyl and propyl;

(t1), (t2), (t3), and (t4) are independently zero or positive integers, preferably 0 or positive integers of from about 1 to about 10 (e.g., 1, 2, 3, 4, 5, 6); and

(a2) and (a3) are independently zero or 1.

The combinations of the bifunctional linkers contemplated within the scope of the present invention include those in which combinations of variables and substituents of the linkers groups are permissible so that such combinations result in stable compounds of Formula (I). For example, when (a3) is zero; Y₁₄ is not linked directly to Y₁₇.

For purposes of the present invention, when values for bifunctional linkers including releasable linkers are positive integers equal to or greater than 2, the same or different bifunctional linkers can be employed.

In one embodiment, Y₁₄₋₁₅ and Y₁₇₋₁₉ are O or NR₂₉; and R₂₁₋₂₉ are independently hydrogen or methyl.

In another embodiment; Y₁₆ is O; Y₁₄₋₁₅ and Y₁₇₋₁₉ are O or NR₂₉; and R₂₁₋₂₉ are hydrogen.

In certain embodiments. L₁₋₃ and L₆₋₉ are independently selected from among:

—(CH₂)ₜ₁—[C(═O)]ₐ₃—,

—(CH₂)ₜ₁Y₁₂—(CH₂)ₜ₂—(Y₁₈)ₐ₂—[C(═O)]ₐ₃—,

—(CH₂CH₂Y₁₇)ₜ₁—[C(═O)]ₐ₃—,

—(CH₂CH₂Y₁₇)ₜ₁(CH₂)ₜ₄—(Y₁₈)ₐ₂—[C(═O)]ₐ₃—,

—[(CH₂CH₂)ₜ₂Y₁₇]ₜ₃(CH₂)ₜ₄—(Y₁₈)ₐ₂—[C(═O)]ₐ₃—,

—(CH₂)ₜ₁—[(CH₂)ₜ₂Y₁₇]ₜ₃(CH₂)ₜ₄—(Y₁₈)ₐ₂—[C(═O)]ₐ₃—,

—(CH₂)ₜ₁(Y₁₇)ₐ₂[C(═O)]ₐ₃(CH₂)ₜ₂—,

—(CH₂)ₜ₁(Y₁₇)ₐ₂[C(═O)]ₐ₃Y₁₄(CH₂)ₜ₂—,

—(CH₂)ₜ₁(Y₁₇)ₐ₂[C(═O)]ₐ₃(CH₂)ₜ₂—Y₁₅—(CH₂)ₜ₃—,

—(CH₂)ₜ₁(Y₁₇)ₐ₂[C(═O)]ₐ₃Y₁₄(CH₂)ₜ₂—Y₁₅—(CH₂)ₜ₃—,

—(CH₂)ₜ₁(Y₁₇)ₐ₂[C(═O)]ₐ₃(CH₂CH₂Y₁₉)ₜ₂(CH₂)ₜ₃—, and

—(CH₂)ₜ₁(Y₁₇)ₐ₂[C(═O)]ₐ₃Y₁₄(CH₂CH₂Y₁₉)ₜ₂(CH₂)ₜ₃—,

wherein

Y₁₄₋₁₅ and Y₁₇₋₁₉ are independently O, or NH;

(t1), (t2), (t3), and (t4) are independently zero or positive integers, preferably 0 or positive integers of from about 1 to about 10 (e.g., 1, 2, 3, 4, 5, 6); and

(a2) and (a3) are independently zero or 1.

Y₁₇, in each occurrence, the same or different, when (t1) or (t3) is equal to or greater than 2.

Y₁₉, in each occurrence, is the same or different, when (t2) is equal to or greater than 2.

In alternative and further embodiments, L₁ is selected from among:

—(CH₂)₄—C(═O)—, —(CH₂)₅—C(═O)—, —(CH₂)₆—C(═O)—,

—CH₂CH₂O—CH₂O—C(═O)—, —(CH₂CH₂O)₂—CH₂O—C(═O)—,

—(CH₂CH₂O)₃—CH₂O—C(═O)—, —(CH₂CH₂O)₂—C(═O)—,

—CH₂CH₂O—CH₂CH₂NH—C(═O)—,

—(CH₂CH₂O)₂—CH₂CH₂NH—C(═O)—,

—CH₂—O—(CH₂CH₂O—CH₂—CH₂NH—C(═O)—,

—CH₂—O—(CH₂CH₂O)₂—CH₂CH₂NH—C(═O)—,

—CH₂—O—CH₂CH₂O—CH₂C(═O)—,

—CH₂—O—(CH₂CH₂O)₂—CH₂C(═O)—,

—(CH₂)₄—C(═O)NH—, —(CH₂)₅—C(═O)NH—, —(CH₂)₅—C(═O)NH—, —(CH₂)₆—C(═O)NH—,

—CH₂CH₂O—CH₂O—C(═O)—NH—,

—(CH₂CH₂O)₂—CH₂O—C(═O)—NH—,

—(CH₂CH₂O)₃—CH₂O—C(═O)—NH—,

—(CH₂CH₂O)₂—C(═O)—NH—,

—CH₂CH₂O—CH₂CH₂NH—C(═O)—NH—,

—(CH₂CH₂O)₂—CH₂CH₂NH—C(═O)—NH—,

—CH₂—O—CH₂CH₂O—CH₂CH₂NH—C(═O)—NH—,

—CH₂—O—(CH₂CH₂O)₂—CH₂CH₂NH—C(═O)—NH—,

—CH₂—O—CH₂CH₂O—CH₂C(═O)—NH—,

—CH₂—O—(CH₂CH₂O)₂—CH₂C(═O)—NH—,

—(CH₂CH₂O)₂—, —CH₂CH₂O—CH₂O—,

—(CH₂CH₂O)₂—CH₂CH₂NH—, —(CH₂CH₂O)₃—CH₂CH₂NH—,

—CH₂CH₂O—CH₂CH₂NH—, —(CH₂CH₂O)₂—CH₂CH₂NH—,

—CH₂—O—CH₂CH₂O—CH₂CH₂NH—,

—CH₂—O—(CH₂CH₂O)₂—CH₂CH₂NH—,

—CH₂—O—CH₂CH₂O—, —CH₂—O—(CH₂CH₂O)₂—,

—(CH₂)₄—, —(CH₂)₃—, —O(CH₂)₂—, —C(═O)O(CH₂)₃—, —C(═O)NH(CH₂)₃—,

—C(═O)(CH₂)₂—, —C(═O)(CH₂)₃—,

—CH₂—C(═O)—O(CH₂)₃—,

—CH₂—C(═O)—NH(CH₂)₃—,

—CH₂—OC(═O)—O(CH₂)₃—,

—CH₂—OC(═O)—NH(CH₂)₃—,

—(CH₂)₂—C(═O)—O(CH₂)₃—,

—(CH₂)₂—C(═O)—NH(CH₂)₃—,

—CH₂C(═O)O(CH₂)₂—O—(CH₂)₂—,

—CH₂C(═O)NH(CH₂)₂—O—(CH₂)₂—,

—(CH₂)₂C(═O)O(CH₂—O—(CH₂)₂—,

—(CH₂)₂C(═O)NH(CH₂)₂—O—(CH₂)₂—,

—CH₂C(═O)O(CH₂CH₂O)₂CH₂CH₂—,

—(CH₂)₂C(═O)O(CH₂CH₂O)₂CH₂CH₂—,

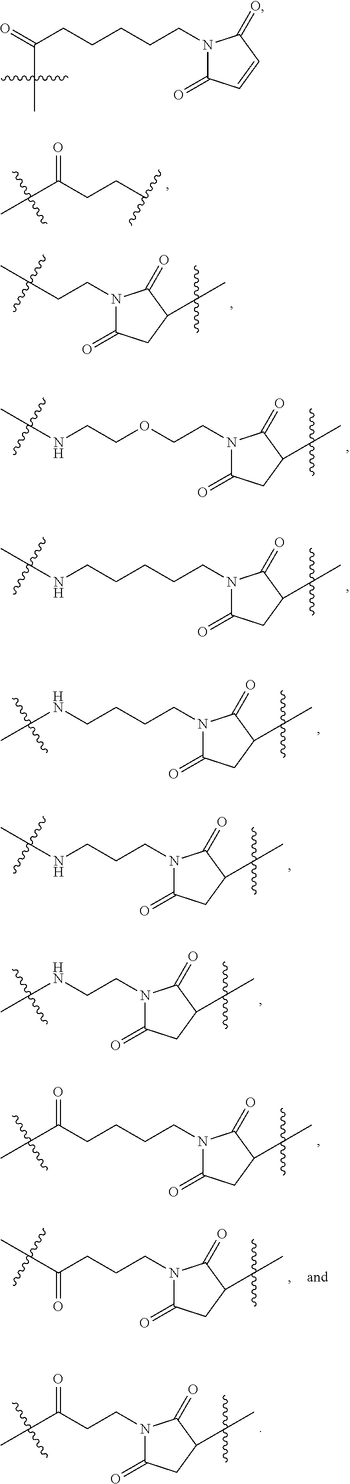

Alternatively, L₂ and L₆₋₇ are independently selected from among:

—(CH₂)₄—C(═O)—, —(CH₂)₅—C(═O)—, —(CH₂)₆—C(═O)—,

—CH₂CH₂O—CH₂O—C(═O)—,

—(CH₂CH₂O)₂—CH₂O—C(═O)—,

—(CH₂CH₂O)₃—CH₂O—C(═O)—,

—(CH₂CH₂O)₂—C(═O)—,

—CH₂CH₂O—CH₂CH₂NH—C(═O)—,

—(CH₂CH₂O)₂—CH₂CH₂NH—C(═O)—,

—CH₂—O—CH₂CH₂O—CH₂CH₂NH—C(═O)—,

—CH₂—O—(CH₂CH₂O)₂—CH₂CH₂NH—C(═O)—,

—CH₂—O—CH₂CH₂O—CH₂C(═O)—,

—CH₂—O—(CH₂CH₂O)₂—CH₂C(═O)—,

—(CH₂)₄—C(═O)NH—, —(CH₂)₅—C(═O)NH—, —(CH₂)₆—C(═O)NH—,

—CH₂CH₂O—CH₂I—C(═O)—NH—,

—(CH₂CH₂O)₂—CH₂O—C(═O)—NH—,

—(CH₂CH₂O)₃—CH₂O—C(═O)—NH—,

—(CH₂CH₂O)₂—C(═O)—NH—,

—CH₂CH₂O—CH₂CH₂NH—C(═O)—NH—,

—(CH₂CH₂O)₂—CH₂CH₂NH—C(═O)—NH—,

—CH₂—O—CH₂CH₂O—CH₂CH₂NH—C(═O)—NH—,

—CH₂—O—(CH₂CH₂O)₂—CH₂CH₂NH—C(═O)—NH—,

—CH₂—O—CH₂CH₂O—CH₂C(═O)—NH—,

—CH₂—O—(CH₂CH₂O)₂—CH₂C(═O)—NH—,

—(CH₂CH₂O)₂—, —CH₂CH₂O—CH₂O—,

—(CH₂CH₂O)₂—CH₂CH₂NH—, —(CH₂CH₂O)₃—CH₂CH₂NH—,

—CH₂CH₂O—CH₂CH₂NH—,

—(CH₂CH₂O)₂—CH₂CH₂NH—,

—CH₂—O—CH₂CH₂O—CH₂CH₂NH—,

—CH₂—O—(CH₂CH₂O)₂—CH₂CH₂NH—,

—CH₂—O—CH₂CH₂O—, —CH₂—O—(CH₂CH₂O)₂—,

—(CH₂)₄—, —(CH₂)₃—, —O(CH₂)₂—, —C(═O)O(CH₂)₃—, —C(═O)NH(CH₂)₃—,

—C(═O)(CH₂)₂—, —C(═O)(CH₂)₃—, —CH₂—C(═O)—O(CH₂)₃—,

—CH₂—C(═O)—NH(CH₂)₃—, —CH₂—OC(═O)—O(CH₂)₃—,

—CH₂—OC(═O)—NH(CH₂)₃—,

—(CH₂)₂—C(═O)—O(CH₂)₃—,

—(CH₂)₂—C(═O)—NH(CH₂)₃—,

—CH₂C(═O)O(CH₂)₂—O—(CH₂)₂—,

—CH₂C(═O)NH(CH₂)₂—O—(CH₂)₂—,

—(CH₂)₂C(═O)O(CH₂)₂—O—(CH₂)₂—,

—(CH₂)₂C(═O)NH(CH₂)₂—O—(CH₂)₂—,

—CH₂C(═O)O(CH₂CH₂O)₂CH₂CH₂—,

—(CH₂)₂C(═O)O(CH₂CH₂O)₂CH₂CH₂—,

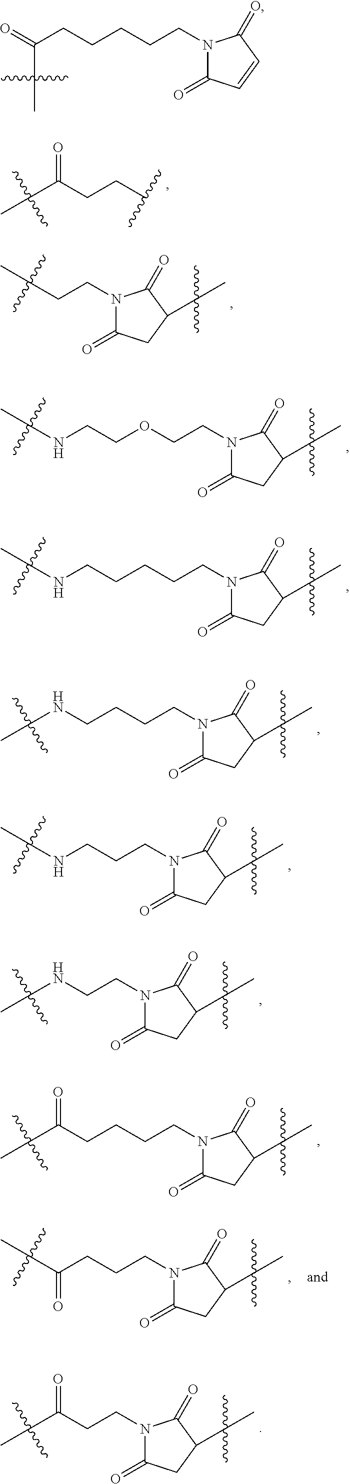

wherein L₂ and L₆₋₇ in each occurrence are independently the same or different when (e), (h) or (i) is equal to or greater than 2.

Alternatively, L₃ and L₈₋₉ are independently selected from among:

—(CH₂)₄—C(═O)—, —(CH₂)₅—C(═O)—, —(CH₂)₆—C(═O)—,

—CH₂CH₂O—CH₂O—C(═O)—, —(CH₂CH₂O)₂—CH₂O'C(═O)—,

—(CH₂CH₂O)₃—CH₂O—C(═O)—, —(CH₂CH₂O)₂—C(═O)—,

—CH₂CH₂O—CH₂CH₂NH—C(═O)—,

—(CH₂CH₂O)₂—CH₂CH₂NH—C(═O)—,

—CH₂—O—CH₂CH₂O—CH₂CH₂NH—C(═O)—,

—CH₂—O—(CH₂CH₂O)₂—CH₂CH₂NH—C(═O)—,

—CH₂—O—CH₂CH₂O—CH₂C(═O)—,

—CH₂—O—(CH₂CH₂O)₂—CH₂C(═O)—,

—(CH₂)₄—C(═O)NH—, —(CH₂)₅—C(═O)NH—, —(CH₂)₆—C(═O)NH—,

—CH₂CH₂O—CH₂O—C(═O)—NH—,

—(CH₂CH₂O)₂—CH₂O—C(═O)—NH—,

—(CH₂CH₂O)₃—CH₂O—C(═O)—NH—,

—(CH₂CH₂O)₂—C(═O)—NH—,

—CH₂CH₂O—CH₂CH₂NH—C(═O)—NH—,

—(CH₂CH₂O)₂—CH₂CH₂NH—C(═O)—NH—,

—CH₂—O—CH₂CH₂O—CH₂CH₂NH—C(═O)—NH—,

—CH₂—O—(CH₂CH₂O)₂—CH₂CH₂NH—C(═O)—NH—,

—CH₂—O—CH₂CH₂O—CH₂C(═O)—NH—,

—CH₂—O—(CH₂CH₂O)₂—CH₂C(═O)—NH—,

—(CH₂CH₂O)₂—, —CH₂CH₂O—CH₂O—,

—(CH₂CH₂O)₂—CH₂CH₂NH—, —(CH₂CH₂O)₃—CH₂CH₂NH—,

—CH₂CH₂O—CH₂CH₂NH—, —(CH₂CH₂O)₂—CH₂CH₂NH—,

—CH₂—O—CH₂CH₂O—CH₂CH₂NH—,

—CH₂—O—(CH₂CH₂O)₂—CH₂CH₂NH—,

—CH₂—O—CH₂CH₂O—, —CH₂—O—(CH₂CH₂O)₂—,

—(CH₂)₄—, —(CH₂)₃—, —O(CH₂)₂—, —C(═O)O(CH₂)₃—,

—C(═O)NH(CH₂)₃—, —C(═O)(CH₂)₂—, —C(═O)(CH₂)₃—,

—CH₂—C(═O)—O(CH₂)₃—,

—CH₂—C(═O)—NH(CH₂)₃—,

—CH₂—OC(═O)—O(CH₂)₃—,

—CH₂—OC(═O)—NH(CH₂)₃—,

—(CH₂)₂—C(═O)—O(CH₂)₃—,

—(CH₂)₂—C(═O)—NH(CH₂)₃—,

—CH₂C(═O)O(CH₂)₂—O—(CH₂)₂—,

—CH₂C(═O)NH(CH₂)₂—O—(CH₂)₂—,

—(CH₂)₂C(═O)O(CH₂)₂—O—(CH₂)₂—,

—(CH₂)₂C(═O)NH(CH₂)₂—O—(CH₂)₂a—,

—CH₂C(═O)O(CH₂CH₂O)₂CH₂CH₂—,

—(CH₂)₂C(═O)O(CH₂CH₂O)₂CH₂CH₂—,

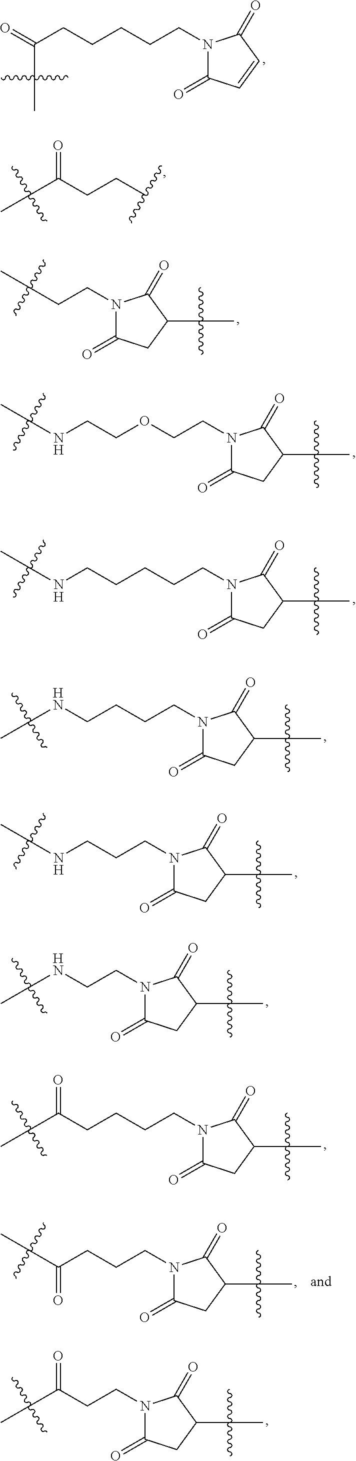

wherein L₃ and L₈₋₉ in each occurrence are independently the same or different when (f), (i) or (j) is equal to or greater than 2.

The combinations of the linker groups contemplated within the scope of the present invention include those in which combinations of variables and substituents of the linkers groups at permissible so that such combinations result in stable compounds of Formula (I). For example, when (a3) is zero, Y₁₇ is not linked directly to Y₁₄ or Y₁₅.

In a further and as an alternative embodiment, bifunctional linkers prior to conjugation to the compound of Formula (I) include amino acids, amino acid derivatives, and peptides. The amino acids can be among naturally occurring and non-naturally occurring amino acids. Derivatives and analogs of the naturally occurring amino acids, as well as various art known non-naturally occurring amino acids (D or L), hydrophobic or nonhydrophobic, are also contemplated to be within the scope of the invention. A suitable non-limiting list of the non-naturally occurring amino acids includes 2-aminoadipic acid, 3-aminoadipic acid, beta-alanine, beta-aminopropionic acid, 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4-aminobutyric acid, desmosine, 2,2-diaminopimelic acid, 2,3-diaminopropionic acid, N-ethylglycine, N-ethylasparagine, 3-hydroxyproline, 4-hydroxyproline, isodesmosine, allo-isoleucine, N4-methylglycine, sarcosine, N-methyl-isoleucine, 6-N-methyl-lysine, N-methylvaline, norvaline, norleucine, and ornithine.

### 3. Bifunctional Spacer Containing a Terminal S: L₄₋₅ Groups

In another aspect of the invention, L₄₋₅, post to being included in compounds of Formula (I), are independently represented by the formula selected from among:

—(CR′₂₁R′₂₂)_{t′1}—[C(Y═Y′₁₆)]_{a′3}(CR′₂₇CR′₂₈)_{t′2}S—,
—(CR′₂₁R′₂₂)_{t′1}Y′₁₄—(CR′₂₃R′₂₄)_{t′2}—(Y′₁₅)_{a′2}—[C(═Y′₁₆)]_{a′3}(CR′₂₇CR′₂₈)_{t′3}S—,
—(CR′₂₁R′₂₂CR′₂₃R′₂₄Y′₁₄)_{t′1}—[C(═Y′₁₆)]_{a′3}(CR′₂₇CR′₂₈)_{t′2}S—,
—(CR′₂₁R′₂₂CR′₂₃R′₂₄Y′₁₄)_{t′1}(CR′₂₅R′₂₆)_{t′2}—(Y′₁₅)_{a′2}—[C(═Y′₁₆)]_{a′3}(CR′₂₇CR′₂₈)_{t′3}S—,
—[(CR′₂₁R′₂₂CR′₂₃R′₂₄)_{t′2}Y′₁₄]_{t′1}(CR′₂₅R′₂₆)_{t′2}—(Y′₁₅)_{a′2}—[C(═Y′₁₆)]_{a′3}(CR′₂₇CR′₂₈)_{t′3}S—,
—(CR′₂₁R′₂₂)_{t′1}—[(CR′₂₃R′₂₄)_{t′2}Y′₁₄]_{t′2}(CR′₂₅R′₂₆)_{t′3}—(Y′₁₅)_{a′2}—[C(═Y′₁₆)]_{a′3}(CR′₂₇CR′₂₈)_{t′4}S—,
—(CR′₂₁R′₂₂)_{t′1}(Y′₁₄)_{a′2}[C(═Y′₁₆)]_{a′3}(CR′₂₃R′₂₄)_{t′2}S—,
—(CR′₂₁R′₂₂)_{t′1}(Y′₁₄)_{a′2}[C(═Y′₁₆)]_{a′3}Y′₁₅(CR′₂₃R′₂₄)_{t′2}S—,
—(CR′₂₁R′₂₂)_{t′1}(Y′₁₄)_{a′2}[C(═Y′₁₆)]_{a′3}(CR′₂₃R′₂₄)_{t′2}—Y′₁₅—(CR′₂₃R′₂₄)_{t′3}S—,
—(CR′₂₁R′₂₂)_{t′1}(Y′₁₄)_{a′2}[C(═Y′₁₆)]_{a′3}Y′₁₄(CR′₂₃R′₂₄)_{t′2}—Y′₁₅—(CR′₂₃R′₂₄)_{t′3}S—,
—(CR′₂₁R′₂₂)_{t′1}(Y′₁₄)_{a′2}[C(═Y′₁₆)]_{a′3}(CR′₂₃R′₂₄CR′₂₅R′₂₆Y′₁₅)_{t′2}(CR′₂₇CR′₂₈)_{t′3}S—,
—(CR′₂₁R′₂₂)_{t′1}(Y′₁₄)_{a′2}[C(═Y′₁₆)]_{a′3}Y′₁₇(CR′₂₃R′₂₄CR′₂₅R′₂₆Y′₁₅)_{t′2}(CR′₂₇CR′₂₈)_{t′3}S—, and

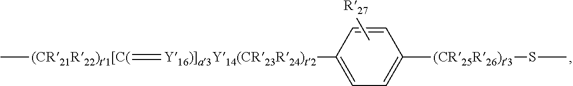

wherein

Y′₁₆ is O, NR′₂₈, or S, preferably O;

Y′₁₄₋₁₅ and Y′₁₇ are independently O, NR′₂₉, or S, preferably O or NR′₂₉;

R′₂₁₋₂₇ are independently selected from the group consisting of hydrogen, hydroxyl, carboxyl, amine, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, heteroalkyls, substituted C₁₋₆ heteroalkyls, C₁₋₆ alkoxy, phenoxy and C₁₋₆ heteroalkoxy, preferably hydrogen, methyl, ethyl and propyl;

R′₂₈₋₂₉ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy, preferably hydrogen, methyl, ethyl and propyl;

(t′1), (t′2), (t′3) and (t′4) are independently zero or positive integers, preferably 0 or positive integers of from about 1 to about 10 (e.g., 1, 2, 3, 4, 5, 6); and

(a′2) and (a′3) are independently zero or 1.

The combinations of the bifunctional spacer groups contemplated within the scope of the present invention include those in which combinations of variables and substituents of the linkers groups are permissible so that such combinations result in stable compounds of Formula (I). For example, when (a′3) is zero, Y′₁₄ is not linked directly to Y′₁₄ or Y′₁₅.

In one preferred embodiment, Y′₁₄₋₁₅ and Y′₁₇ are O or NR′₂₉; and R′₂₁₋₂₉ are independently hydrogen, or methyl.

In a further preferred embodiment. Y′₁₆ is O; Y′₁₄₋₁₅ and Y′₁₇ are O or NR′₂₉; and R′₂₁₋₂₉ are hydrogen.

In certain embodiments, L₁₋₃ and L₆₋₉ are independently selected from among:

—(CH₂)_{t′1}—[C(═O)]_{a′3}(CH₂)_{t′2}S—,

—(CH₂)_{t′1}Y′₁₄—(CH₂)_{t′2}—(Y′₁₅)_{a′2}—[C(═O)]_{a′3}(CH₂)_{t′3}S—,

—(CH₂CH₂Y′₁₄)_{t′1}—[C(═O)]_{a′3}(CH₂)_{t′2}S—,

—(CH₂CH₂Y′₁₄)_{t′1}(CH₂)_{t′2}—(Y′₁₅)_{a′2}—[C(═O)]_{a′3}(CH₂)_{t′3}S—,

—[(CH₂CH₂)_{t′2}Y′₁₄]_{t′1}(CH₂)_{t′2}—(Y′₁₅)_{a′2}—[C(═O)]_{a′3}(CH₂)_{t′3}S—,

—(CH₂)_{t′1}—[(CH₂)_{t′2}Y′₁₄]_{t′2}(CH₂)_{t′3}—(Y′₁₅)_{a′2}—[C(═O)]_{a′3}(CH₂)_{t′4}S—,

—(CH₂)_{t′1}(Y′₁₄)_{a′2}[C(═O)]_{a′3}(CH₂)_{t′2}S—,

—(CH₂)_{t′1}(Y′₁₄)_{a′2}[C(═O)]_{a′3}Y′₁₅(CH₂)_{t′2}S—,

—(CH₂)_{t′1}(Y′₁₄)_{a′2}[C(═O)]_{a′3}(CH₂)_{t′2}—Y′₁₅—(CH₂)_{t′3}S—,

—(CH₂)_{t′1}(Y′₁₄)_{a′2}[C(═O)]_{a′3}Y′₁₄(CH₂)_{t′2}—Y′₁₅—(CH₂)_{t′3}S—,

—(CH₂)_{t′1}(Y′₁₄)_{a′2}[C(═O)]_{a′3}(CH₂CH₂Y′₁₅)_{t′2}(CH₂)_{t′3}S—, and

—(CH₂)_{t′1}(Y′₁₄)_{a′2}[C(═O)]_{a′3}Y′₁₇(CH₂CH₂Y′₁₅)_{t′2}(CH₂)_{t′3}S—,

wherein

Y′₁₄₋₁₅ and Y′₁₇ are independently O, or NH;

(t′1), (t′2), (t′3), and (t′4) are independently zero or positive integers, preferably 0 or positive integers of from about 1 to about 10 (e.g., 1, 2, 3, 4, 5, 6); and

(a′2) and (a′3) are independently zero or 1.

Y′₁₄, in each occurrence, is the same or different, when (t′1) or (t′2) is equal to or greater than 2.

Y′₁₅, in each occurrence, is the same or different, when (t′2) is equal to or greater than 2.

For poses of the present invention, when values for bifunctional spacers including releasable linkers are positive integers equal to or greater than 2, the same or different bifunctional linkers can be employed.

In a further embodiment and as an alternative embodiment, L₄ is selected from among:

—(CH)₆—S—, —(CH)₅—S—, —(CH)₄—S—, —(CH)₃—S—, —(CH)₂—S—,

—(CH₂)₄—C(═O)NH—CH(COOH)CH₂S—,

—(CH₂)₅—C(═O)NH—CH(COOH)CH₂S—,

—(CH₂)₆—C(═O)NH—CH(COOH)CH₂S—,

—CH₂CH₂O—CH₂O—C(═O)NH—CH)COOH)CH₂S—,

—(CH₂CH₂O)₂—CH₂O—C(═O)NH—CH(COOH)CH₂S—,

—(CH₂CH₂O)₃—CH₂O—C(═O)NH—CH(COOH)CH₂S—,

—(CH₂CH₂O)₂—C(═O)NH—CH(COOH)CH₂S—,

—CH₂CH₂O—CH₂CH₂NH—C(═O)NH—CH(COOH)CH₂S—,

—(CH₂CH₂O)₂—CH₂CH₂NH—C(═O)NH—CH(COOH)CH₂S—,

—CH₂—O—CH₂CH₂O—CH₂CH₂NH—C(═O)NH—CH(COOH)CH₂S—,

—CH₂—O—(CH₂CH₂O)₂—CH₂CH₂NH—C(I═O)NH—CH(COOH)CH₂S—,

—CH₂—O—CH₂CH₂O—CH₂C(═O)NH—CH(COOH)CH₂S—,

—CH₂—O—)CH₂CH₂O)₂—CH₂C(═O)NH—CH(COOH)CH₂S—,

—(CH₂)₄—C(═O)NHCH(COOH)CH₂S—,

—(CH₂CH₂O)₂CH₂C(═O)NH—CH(COOH)CH₂S—,

—CH₂CH₂O—CH₂OC(═O)NH—CH(COOH)CH₂S—,

—(CH₂CH₂O)₂—CH₂CH₂NHC(═O)CH(NH₂)CH₂S—,

—(CH₂CH₂O)₃—CH₂CH₂NHC(═O)CH(NH₂)CH₂S—,

—CH₂CH₂O—CH₂CH₂NHC(═O)CH(NH₂)CH₂S—,

—(CH₂CH₂O)₂—CH₂CH₂NHC(═O)CH(NH₂)CH₂S—,

—CH₂—O—CH₂CH₂O—CH₂CH₂NHC(═O)CH(NH₂)CH₂S—,

—CH₂—O—(CH₂CH₂O)₂—CH₂CH₂NHC(═O)CH(NH₂CH₂S—,

—CH₂—O—CH₂CH₂O—CH₂C(═O)NHCH(COOH)CH₂S—, and

—CH₂—O—(CH₂CH₂O)₂—CH₂C(═O)NHCH(COOH)CH₂S—.

In a further embodiment and as an alternative embodiment, L₅ is selected from among:

—(CH)₆—S—, —(CH)₅—S—, —(CH)₄—S—, —(CH)₃—S—, —(CH)₂—S—,

—(CH₂CH₂O)—CH₂CH₂S—,

—(CH₂CH₂O)₂—CH₂CH₂S—,

—(CH₂)₄—C(═O)NH—CH(COOH)CH₂S—,

—(CH₂)₅—C(═O)NH—CH(COOH)CH₂S—,

—(CH₂)₆—C(═O)NH—CH(COOH)CH₂S—,

—CH₂CH₂O—CH₂O—C(═O)NH—CH(COOH)CH₂S—,

—(CH₂CH₂O)₂—CH₂O—C(═O)NH—CH(COOH)CH₂S—,

—(CH₂CH₂O)₃—CH₂O—C(═O)NH—CH(COOH)CH₂S—,

—(CH₂CH₂O)₂—C(═O)NH—CH(COOH)CH₂S—,

—CH₂CH₂O—CH₂CH₂NH—C(═O)NH—CH(COOH)CH₂S—,

—(CH₂CH₂O)₂—CH₂CH₂NH—C(═O)NH—CH(COOH)CH₂S—,

—CH₂—O—CH₂CH₂O—CH₂CH₂NH—C(═O)NH—CH(COOH)CH₂S—,

—CH₂—O—(CH₂CH₂O)₂—CH₂CH₂NH—C(═O)NH—CH(COOH)CH₂S—,

—CH₂—O—CH₂CH₂O—CH₂C(═O)NH—CH(COOH)CH₂S—,

—CH₂—O—(CH₂CH₂O)₂—CH₂C(═O)NH—CH(COOH)CH₂S—,

—(CH₂)₄—C(═O)NHCH(COOH)CH₂S—,

—(CH₂CH₂O)₂CH₂C(═O)NH—CH(COOH)CH₂S—,

—CH₂CH₂O—CH₂OC(═O)NH—CH(COOH)CH₂S—,

—(CH₂CH₂O)₂—CH₂CH₂NHC(═O)CH(NH₂)CH₂S—,

—(CH₂CH₂O)₃—CH₂CH₂NHC(═O)CH(NH₂)CH₂S—,

—CH₂CH₂O—CH₂CH₂NHC(═O)CH(NH₂)CH₂S—,

—(CH₂CH₂O)₂—CH₂CH₂NHC(═O)CH(NH₂)CH₂S—,

—CH₂—O—CH₂CH₂O—CH₂CH₂NHC(═O)CH(NH₂)CH₂S—,

—CH₂—O—(CH₂CH₂O)₂—CH₂CH₂NHC(═O)CH(NH₂)CH₂S—,

—CH₂—O—CH₂CH₂O—CH₂C(═O)NHCH(COOH)CH₂S—, and

—CH₂—O—(CH₂CH₂O)₂—CH₂C(═O)NHCH(COOH)CH₂S—.

### 4. Leaving Groups and Functional Groups

In some aspects, suitable leaving groups include, without limitations to, halogen (Br, Cl), activated carbonate, carbonyl imidazole, cyclic imide thione, isocyanate, M-hydroxysuccinimidyl, para-nitrophenoxy, N-hydroxyphtalimide, N-hydroxybenzotriazolyl, imidazole, tosylate, mesylate, tresylate, nosylate, C₁-C₆ alkyloxy, C₁-C₆ alkanoyloxy, arylcarbonyloxy, ortho-nitrophenoxy, N-hydroxybenzotriazolyl, imidazole, pentafluorophenoxy, 1,3,5-trichlorophenoxy, and 1,3,5-trifluorophenoxy or other suitable leaving groups, as will be apparent to those of ordinary skill.

For purposes of the present invention, leaving groups are to be understood as those groups which are capable of reacting with a nucleophile found on the desired target, i.e. a biologically active agent, a diagnostic agent, a targeting moiety, a bifunctional spacer, intermediate, etc. The targets thus contain a group for displacement, such as OH, NH₂ or SH groups found on oligonucleotides modified with a spacer-SH, a spacer-NH₂, or a spacer-OH, proteins, peptides, enzymes, naturally or chemically synthesized therapeutic molecules such as doxorubicin, and spacers such as mono-protected diamines.

In some preferred embodiments, functional groups to link compounds of Formula (I) to biologically active agents include maleimidyl, vinyl, residues of sulfone, amino, carboxy, mercapto, hydrazide, carbazate and the like which can be further conjugated to a biologically active group.

In yet some preferred embodiments of the invention, the leaving groups can be selected from among H, OH, methoxy, tert-butoxy, N-hydroxysuccinimidyl and maleimidyl.

### 5. Biologically Active Agents

In another aspect of the invention, a wide variety of biologically active agents are contemplated in the compounds of Formula (I) described herein. The biologically active agents include pharmaceutically active compounds, enzymes, proteins, nucleic acids (e.g., oligonucleotides), antibodies, monoclonal antibodies, single chain antibodies and peptides. Alternatively, the compounds of Formula (I) contain a biologically active agent which includes amine-, hydroxyl-, or thiol-containing compounds.

For purposes of the present invention, it shall be understood to mean that the pharmaceutically active compounds include small molecular weight molecules. Typically, the pharmaceutically active compounds have a molecular weight of less than about 1,500 daltons.

A non-limiting list of such compounds includes camptothecin and analogs such as SN38 or irinotecan, hydroxyl- or thiol-topoisomerase I inhibitors, taxanes and paclitaxel derivatives, nucleosides including AZT and acyclovir, anthracycline compounds including daunorubicin and doxorubicin, related anti-metabolite compounds including Ara-C (cytosine arabinoside) and gemcitabine, etc.

Alternatively, biologically active agents can include cardiovascular agents, anti-neoplastic, anti-infective, anti-fungal such as nystatin and amphotericin B, anti-anxiety agents, gastrointestinal agents, central nervous system-activating agents, analgesic, fertility agents, contraceptive agents, anti-inflammatory agents, steroidal agents, anti-urecemic agents, vasodilating agents, and vasoconstricting agents, etc. It is to be understood that other biologically active materials not specifically mentioned, but having suitable amine-, hydroxyl- or thiol-containing groups, are also intended and are within the scope of the present invention.

The biologically active compounds are suitable for medicinal or diagnostic use in the treatment of animals, e.g., mammals, including humans, for conditions for which such treatment is desired.

The only limitations on the types of the biologically active agents suitable for inclusion herein is that there is available al least one chemically reactive functional moiety such as amine, hydroxyl, or thiol to link to the compounds of Formula (I) and that there is not substantial loss of bioactivity in the form conjugated to the compounds of Formula (I) described herein. Alternatively, compounds suitable for incorporation into the compounds of the present invention, may be active after hydrolytic release from the linked compound, or not active after hydrolytic release but which will become active after undergoing a further chemical process/reaction. For example, an anticancer drug that is delivered to the bloodstream by the delivery system, may remain inactive until entering a cancer or tumor cell, whereupon it is activated by the cancer or tumor cell chemistry, e.g., by an enzymatic reaction unique to that cell.

In one preferred embodiment, the biologically active agent is a biologically active agent containing neutral or negative charges. The biologically active agents include nucleic acids such as an oligonucleotide, and negatively charged pharmaceutically active compounds. The negatively charged pharmaceutically active compounds include small molecules such as those having an average molecular weight of less than about 1,500 daltons.

In more preferred embodiment, the biologically active agent includes an oligonucleotide.

### 6. Nucleic Acids/Oligonucleotides

The compounds described herein can be used for delivering various nucleic acids (e.g., oligonucleotides) into cells or tissues, and preferably into the cytoplasm and the nucleus. The nucleic acids include plasmids and oligonucleotides. Preferably, the compounds described herein are used for delivery of oligonucleotides.

In order to more fully appreciate the scope of the present invention, the following terms are defined. The artisan will appreciate that the terms, “nucleic acid” or “nucleotide” apply to deoxyribonucleic acid (“DNA”), ribonucleic acid, (“RNA”) whether single-stranded or double-stranded, unless otherwise specified, and to any chemical modifications or analogs thereof, for example, locked nucleic acids (INA). The artisan will readily understand that by the term “nucleic acid,” included are polynucleic acids, derivates, modifications and analogs thereof. An “oligonucleotide” is generally a relatively short polynucleotide, e.g., ranging in size from about 2 to about 200 nucleotides, or preferably from about to about 50 nucleotides, or more preferably from 8 to 20 or 15-28 in length. The oligonucleotides according to the invention are generally synthetic nucleic acids, and are single stranded, unless otherwise specified. The terms, “polynucleotide” and “polynucleic acid” may also be used synonymously herein.

The oligonucleotides (analogs) are not limited to a single species of oligonucleotide but, instead, are designed to work with a wide variety of such moieties, it being understood that linkers can attach to one or more of the 3′- or 5′-terminals, usually PO₄ or SO₄ groups of a nucleotide. The nucleic acids molecules contemplated can include a phosphorothioate internucleotide linkage modification, sugar modification, nucleic acid base modification and/or phosphate backbone modification. The oligonucleotides can contain natural phosphorodiester backbone or phosphorothioate backbone or any other modified backbone analogues such as LNA (Locked Nucleic Acid), PNA (nucleic acid with peptide backbone), CpG oligomers, and the like, such as those disclosed at Tides 2002, Oligonucleotide and Peptide Technology Conferences, May 6-8, 2002, Las Vegas, Nev. and Oligonucleotide & Peptide Technologies, 18 & 19 Nov. 2003, Hamburg, Germany, the contents of which are incorporated herein by reference.

Modifications to the oligonucleotides contemplated by the invention include, for example, the addition or substitution of functional moieties that incorporate additional charge, polarizability, hydrogen bonding, electrostatic interaction, and functionality to an oligonucleotide. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodouracil, backbone modifications, methylations, base-pairing combinations such as the isobases isocytidine and isoguanidine, and analogous combinations. Oligonucleotides contemplated within the scope of the present invention can also include 3′ and/or 5′ cap structure.

For purposes of the present invention, “cap structure” shall be understood to mean chemical modifications, which have been incorporated at either terminus of the oligonucleotide. The cap can be present at the 5′-terminus (5′-cap) or at the 3′-terminus (3′-cap) or can be present on both termini. A non-limiting example of the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety. Details are described in WO 97/26270, incorporated by reference herein. The 3′-cap can include for example 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties. See also Beaucage and Iyer; 1993, Tetrahedron 49, 1925; the contents of which are incorporated by reference herein.

A non-limiting list of nucleoside analogs have the structure:

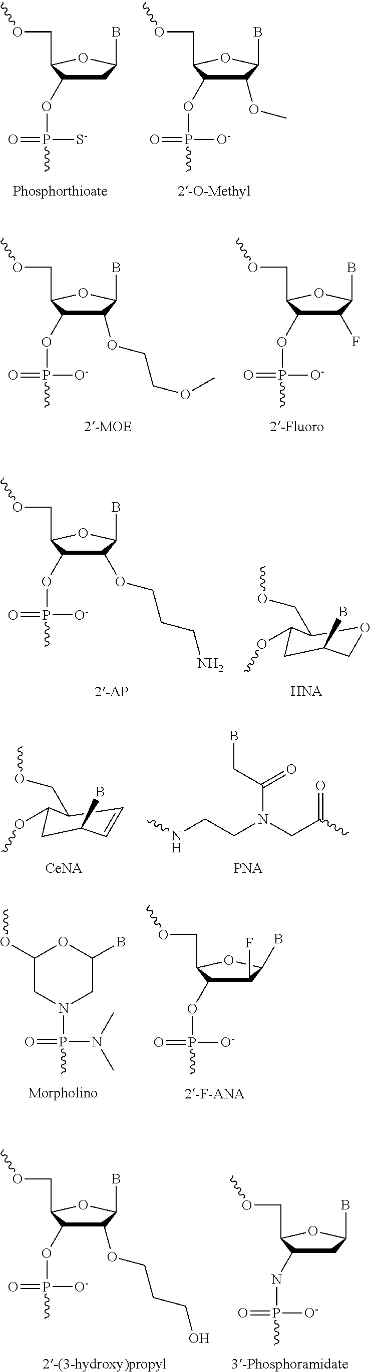

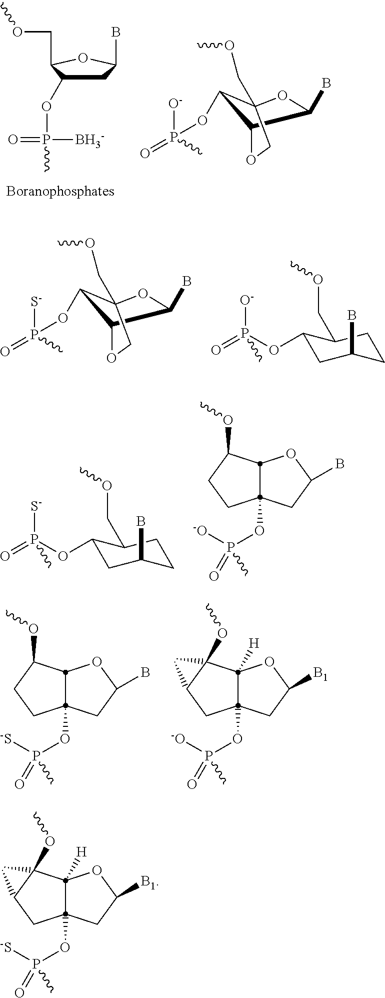

See more examples of nucleoside analogues described in Freier & Altmann; Nucl. Acid Res., 1997, 25, 4429-4443 and Uhlmann; Curr. Opinion in Drug Development, 2000, 3(2), 293-213, the contents of each of which are incorporated herein by reference.

The term “antisense,” as used herein, refers to nucleotide sequences which are complementary to a specific DNA or RNA sequence that encodes a gene product or that encodes a control sequence. The term “antisense strand” is used reference to a nucleic acid strand that is complementary to the “sense” strand. In the normal operation of cellular metabolism, the sense strand of a DNA molecule is the strand that is transcribed into messenger RNA (“mRNA”) during transcription. The sense strand thus serves as a template for synthesis of a messenger RNA (“mRNA”) transcript (an antisense strand) which, in turn, directs synthesis of any encoded gene product. Antisense nucleic acid molecules may be produced by any art-known methods, including synthesis by ligating the gene(s) of interest in a reverse orientation to a viral promoter which permits the synthesis of a complementary strand. Once introduced into a cell, this transcribed strand combines with natural sequences produced by the cell to form duplexes. These duplexes then block either the further transcription or translation. The designations “negative” or (−) are also art-known to refer to the antisense strand, and “positive” or (+) are also art-known to refer to the sense strand.

For purposes of the present invention, “complementary” shall be understood to mean that a nucleic acid sequence forms hydrogen bond(s) with another nucleic acid sequence. A percent complementarity indicates the percentage of contiguous residues in a nucleic acid molecule which can form hydrogen bonds, i.e., Watson-Crick base pairing, with a second nucleic acid sequence, i.e., 5, 6, 7, 8, 9, 10 out of 10 being 50%, 60%, 70%, 80%, 90%, and 100% complementary. “Perfectly complementary” means that all the contiguous residues of a nucleic acid sequence form hydrogen bonds with the same number of contiguous residues in a second nucleic acid sequence.

The nucleic acids (such as one or more oligonucleotides (same or different) or oligonucleotide derivatives) useful in the compounds described herein can include from about 5 to about 1000 nucleic acids, and preferably relatively short polynucleotides, e.g., ranging in size preferably from about 8 to about 50 nucleotides in length (e.g., about 8, 9, 10, 11, 12, 13, 14, 15, 16, 17, 18, 19, 20, 21, 22, 23, 24, 25, 26, 27, 28, 29, or 30).

In one aspect of useful nucleic acids contemplated in the compounds described herein, oligonucleotides and oligodeoxynucleotides with, natural phosphorodiester backbone or phosphorothioate backbone or any other modified backbone analogues include;

LNA (Locked Nucleic Acid);

PNA (nucleic acid with peptide backbone);

short interfering RNA (siRNA);

microRNA (miRNA);

nucleic acid, with peptide backbone (PNA);

phosphorodiamidate morpholino oligonucleotides (PMO);

tricyclo-DNA;

decoy ODN (double stranded oligonucleotide);

catalytic RNA sequence (RNAi);

ribozymes;

aptamers;

spiegelmers (L-conformational oligonucleotides);

CpG oligomers, and the like, such as those disclosed at:

Tides 2002, Oligonucleotide and Peptide Technology Conferences, May 6-8, 2002, Las Vegas, Nev. and Oligonucleotide & Peptide Technologies, 18 & 19 Nov. 2003, Hamburg, Germany, the contents of which are incorporated herein by reference.

Oligonucleotides according to the invention can also optionally include any suitable art-known nucleotide analogs and derivatives, including those listed by Table 1, below:

TABLE 1
Representative Nucleotide Analogs Anal Derivatives

| | |
|---|---|
| 4-acetylcytidine | 5-methoxyaminomethyl-2-thiouridine |
| 5-(carboxyhydroxymethyl)uridine | beta, D-mannosylqueuosine |
| 2′-O-methylcytidine | 5-methoxycarbonylmethyl-2- |
| | thiouridinee |
| 5-methoxycarbonylmethyluridine | 5-carboxymethylaminomethyl-2- |
| | thiouridine |
| 5-methoxyuridine | 5-carboxymethylaminomethyluridine |
| Dihydrouridine | 2-methylthio-N6- |
| | isopentenyladenosine |
| 2′-O-methylpseudouridine | N-[(9-beta-D-ribofuranosyl-2- |
| | methylthiopurine-6- |
| | yl)carbamoyl]threonine |
| D-galactosylqueuosine | N-[(9-beta-D-ribofuranosylpurine-6- |
| | yl)N-methylcarbamoyl]threonine |
| 2′-O-methylguanosine | uridine-5-oxyacetic acid-methylester |
| 2′-halo-adenosine | 2′-halo-cytidine |
| 2′-halo-guanosine | 2′-halo-thymine |
| 2′-halo-uridine | 2′-halo-methylcytidine |
| 2′-amino-adenosine | 2′-amino-cytidine |
| 2′-amino-guanosine | 2′-amino-thymine |
| 2′-amino-uridine | 2′-amino-methylcytidine |
| Inosine | uridine-5-oxyacetic acid |
| N6-isopentenyladenosine | Wybutoxosine |
| 1-methyladenosine | Pseudouridine |
| 1-methylpseudouridine | Queuosine |
| 1-methylguanosine | 2-thiocytidine |
| 1-methylinosine | 5-methyl-2-thiouridine |
| 2,2-dimethylguanosine | 2-thiouridine |
| 2-methyladenosine | 4-thiouridine |
| 2-methylguanosine | 5-methyluridine |
| 3-methylcytidine | N-[(9-beta-D-ribofuranosylpurine-6- |
| | yl)-carbamoyl]threonine |
| 5-methylcytidine | 2′-O-methyl-5-methyluridine |
| N6-methyladenosine | 2′-O-methyluridine |
| 7-methyguanosine | Wybutosine |
| 5-methylaminomethyluridine | 3-(3-amino-3-carboxy-propyl)uridine |
| Locked-adenosine | Locked-cytidine |
| Locked-guanosine | Locked-thymine |
| Locked-uridine | Locked-methylcytidine |

In one preferred aspect, the target oligonucleotides contemplated in the compounds described herein includes, for example, but is not limited to, oncogenes, pro-angiogenesis pathway genes, pro-cell proliferation pathway genes, viral infectious agent genes, and pro-inflammatory pathway genes.

In one preferred embodiment, the oligonucleotide contemplated in the compounds described herein is involved in targeting tumor cells or downregulating a gene or protein expression associated with tumor cells and/or the resistance of tumor cells to anticancer therapeutics. For example, antisense oligonucleotides for downregulating any art-known cellular proteins associated with cancer, e.g., BCL-2 can be used for the present invention. See U.S. patent application Ser. No. 10/822,205 filed Apr. 9, 2004, the contents of which are incorporated by reference herein. A non-limiting list of preferred therapeutic oligonucleotides includes antisense bcl-2 oligonucleotides, antisense HIF-1α oligonucleotides, antisense survivin oligonucleotides, antisense ErbB3 oligonucleotides, antisense PIK3CA oligonucleotides, antisense HSP27 oligonucleotides, antisense androgen receptor oligonucleotides, antisense Gli2 oligonucleotides, and antisense beta-catenin oligonucleotides.

More preferably, the oligonucleotides according to the invention described herein include phosphorothioate backbone and LNA.

In one embodiment, the oligonucleotide can be, for example, antisense survivin LNA oligomers, antisense ErbB3 LNA oligomers, or HIF1-α LNA oligomers.

In another preferred embodiment, the oligonucleotide can be, for example, an oligonucleotide that has the same or substantially similar nucleotide sequence as does Genasense® (a/k/a oblimersen sodium, produced by Genta Inc., Berkeley Heights, N.J.). Genasense® is an 18-mer phosphorothioate antisense oligonucleotide (SEQ ID NO: 4), that is complementary to the first six codons of the initiating sequence of the human bcl-2 mRNA (human bcl-2 mRNA is art-known, and is described, e.g., as SEQ ID NO: 19 in U.S. Pat. No. 6,414,134, incorporated by reference herein).

Preferred embodiments contemplated include:

(i) antisense Survivin LNA oligomer (SEQ ID NO: 1)
^{m}Cₛ-Tₛ-^{m}Cₛ-Aₛ₋aₛ-tₛ-cₛ-aₛ-tₛ-gₛ-gₛ-^{m}Cₛ-Aₛ-Gₛ-c;
where the upper case letter represents LNA, the “s” represents a phosphorothioate backbone;

(ii) antisense Bcl2 siRNA:
SENSE 5′-gcaugcggccucuguuugadTdT-3′ (SEQ ID NO: 2)
ANTISENSE 3′-dTdTcguacgccggagacaaacu-5′ (SEQ ID NO: 3)
where dT represents DNA;

(iii) Genasense (phosphorothioate antisense oligonucleotide); (SEQ ID NO: 4)
tₛ-cₛ-tₛ-cₛ₋cₛ-cₛ-aₛ-gₛ-cₛ-gₛ-tₛ-gₛ-cₛ-gₛ-cₛ-cₛ-cₛ-aₛ-t
where the lower case letter represents DNA and “s” represent phosphorothioate backbone;

(iv) antisense HIF1α LNA oligomer (SEQ ID NO: 5)
TₛGₛGₛcₛaₛaₛgₛcₛaₛtₛcₛcₛTₛGₛTₛa
where the upper case letter represents LNA and the “s” represents phosphorothioate backbone,

(v) antisense ErbB3 LNA oligomer (SEQ ID NO: 6)
TₛAₛGₛcₛcₛtₛgₛtₛcₛaₛcₛtₛtₛ^{Me}CₛTₛ^{Me}Cₛ
where the upper case letter represents LNA and the “s” represents phosphorothioate backbone.

(vi) antisense ErbB3 LNA oligomer (SEQ ID NO: 7)
Gₛ^{Me}CₛTₛcₛcₛaₛgₛaₛcₛaₛtₛcₛaₛ^{Me}CₛTₛ^{Me}C
where the upper case letter represents LNA and the “s” represents phosphorothioate backbone.

(vii) antisense PIK3CA LNA oligomer (SEQ ID NO: 8)
AₛGₛ^{Me}CₛcₛaₛtₛtₛcₛaₛtₛtₛcₛcₛAₛ^{Me}Cₛ^{Me}C
where the upper case letter represents LNA and the “s” represents phosphorothioate backbone.

(viii) antisense PIK3CA LNA oligomer (SEQ ID NO: 9)
TₛTₛAₛtₛtₛgₛtₛgₛcₛaₛtₛcₛtₛ^{Me}CₛAₛG
where the upper case letter represents LNA and the “s” represents phosphorothioate backbone.

(ix) antisense HSP27 LNA oligomer (SEQ ID NO: 10)
CₛGₛTₛgₛtₛaₛtₛtₛtₛcₛcₛgₛcₛGₛTₛG
where the upper case letter represents LNA and the “s” represents phosphorothioate backbone.

(x) antisense HSP27 LNA oligomer (SEQ ID NO: 11)
GₛGₛ^{Me}CₛaₛcₛaₛgₛcₛcₛaₛgₛtₛgₛGₛ^{Me}CₛG
where the upper case letter represents LNA and the “s” represents phosphorothioate backbone.

(xi) antisense Androgen Receptor LNA oligomer (SEQ ID NO: 12)
^{Me}Cₛ^{Me}Cₛ^{Me}CₛaₛaₛgₛgₛcₛaₛcₛtₛgₛcₛAₛGₛA
where the upper case letter represents LNA and the “s” represents phosphorothioate backbone.

(xii) antisense Androgen Receptor LNA oligomer (SEQ ID NO: 13)
Aₛ^{Me}Cₛ^{Me}CₛaₛaₛgₛtₛtₛtₛcₛtₛtₛcₛAₛGₛ^{Me}C
where the upper case letter represents LNA and the “s” represents phosphorothioate backbone.

(xiii) antisense GLI2 LNA oligomer (SEQ ID NO: 14)
^{Me}CₛTₛ^{Me}CₛcₛtₛtₛgₛgₛtₛgₛcₛaₛgₛTₛ^{Me}CₛT
where the upper case letter represents LNA and the “s” represents phosphorothioate backbone.

(xiv) antisense GLI2 LNA oligomer (SEQ to NO: 15)
Tₛ^{Me}CₛAₛgₛaₛtₛtₛcₛaₛaₛaₛcₛ^{Me}Cₛ^{Me}CₛA
where the upper case letter represents LNA and the “s” represents phosphorothioate backbone

(xv) antisense beta-catenin LNA oligomer (SEQ ID NO: 16)
GₛTₛGₛtₛtₛcₛtₛaₛcₛaₛcₛcₛaₛTₛTₛA
where the upper case letter represents LNA and the “s” represents phosphorothioate backbone.

Lower case letters represent DNA units, bold upper case letters represent LNA such as β-D-oxy-LNA units. All cytosine bases in the LNA monomers are 5-methylcytosine. Subscript “s” represents phosphorothioate linkage.

LNA includes 2′-O,4′-C methylene bicyclonucleotide as shown below:

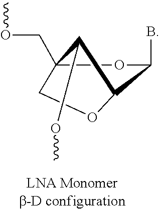

See detailed description of Survivin LNA disclosed in U.S. patent application Ser. Nos. 11/272,124, entitled “LNA Oligonucleotides and the Treatment or Cancer” and 10/776,934, entitled “Oligomeric Compounds for the Modulation Survivin Expression”, the contents of each of which is incorporated herein by reference. See also U.S. Pat. No. 7,582,190 and U.S. Patent Publication No. 2004/0096848 for HIF-1α modulation; U.S. Patent Publication No. 2008/0318894 and PCT/US09/063357 for ErbB3 modulation; U.S. Patent Publication No. 2009/0192110 for PIK3CA modulation; PCT/IB09/052860 for HSP27 modulation; U.S. Patent Publication No. 2009/0181916 for Androgen Receptor modulation; and U.S. Provisional Application No. 61/081,135 and PCT Application No. PCT/IB09/006407, entitled “RNA Antagonists Targeting GLI2”; and U.S. Patent Publication Nos. 2009/0005335 and 2009/0203137 for Beta Catenin modulation; the contents of each which are also incorporated herein by reference. Additional examples of suitable target genes are described in WO 03/74654, PCT/US03/05028, and U.S. patent application Ser. No. 10/923,536, the contents of which are incorporated by reference herein.

The oligonucleotide molecule employed in the conjugates described herein can be modified with (CH₂)_{w} hydroxyl linkers, (CH₂)_{w} amino linkers, or (CH₂)_{w} sulfhydryl linkers at 5′ or 3′ end of the oligonucleotides, where (w) in this aspect is a positive integer of preferably from about 1 to about 10 (1, 2, 3, 4, 5, 6, 7, 8, 9, 10), preferably 6.

In another embodiment, the compounds described herein can include oligonucleotides modified with a hindered ester-containing (CH₂)_{w} hydroxyl linker, a hindered ester-containing (CH₂)_{w} amino linker and a hindered ester-containing (CH₂)_{w} sulfhydryl linker at 5′ or 3′ end of the oligonucleotides, where (w) in this aspect is a positive integer of preferably from about 1 to about 10, preferably about 6. See PCT/USO7/78597 entitled “Hindered Ester-Based Biodegradable Linkers For Oligonucleotide Delivery” and PCT/USO7/78593 entitled “Polyaklylene Oxides Having Hindered Ester-Based Biodegradable Linkers”, the contents of each of which are incorporated by reference. The compounds of Formula (I) can release the oligonucleotides without amine tail. For example, the oligonucleotides prior to the conjugation can include a hindered ester having the structure:

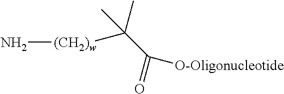

wherein (w) is a positive integer from about 1 to about 10, preferably about 6.

The oligonucleotides prior to the conjugation to the compounds described herein include (CH₂)_{w} sulfhydryl linkers (thio oligonucleotides) at 5′ or 3′ end of the oligonucleotides, where (w) in this aspect is a positive integer of preferably from about 1 to about 10, preferably 6. The thio oligonucleotides have the structure SH—(CH₂)_{w}-Oligonucleotide. The polymeric compounds can release the oligonucleotides without thiol tail. For example, the thio oligonucleotides can also include a hindered ester having the structure:

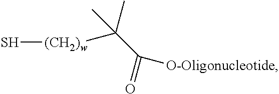

wherein (w) is a positive integer from about 1 to about 10, preferably about 6.

In one embodiment, 5′ end of sense strand of siRNA is modified. For example, siRNA employed in the compounds described herein is modified with a 5′-C₆—SH. One particular embodiment of the present invention employs Bcl2-siRNA having the sequence of

SENSE 5′-(SH—C₆)GCAUGCGGCCUCUGUUUGAdTdT-3′

ANTISENSE 3′-dTdTCGUACGCCGGAGACAAACU-5′.

Additional examples of the modified oligonucleotides include:

(i) Genasense modified with a C₆—SH tail:
5′-HS—C₆-ₛtₛcₛtₛcₛcₛcₛaₛgₛcₛgₛtₛgₛcₛgₛcₛcₛaₛr-3′

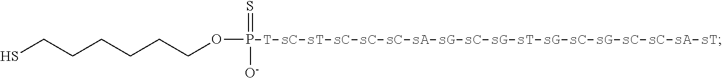

antisense HIF1α LNA oligomer modified with a C₆—SH tail:
5′-HS—C₆-ₛTₛGₛGₛcₛaₛaₛgₛcₛaₛtₛcₛcₛTₛGₛTₛa-3′;

(iii) antisense Survivin LNA oligomer modified with a C₆—SH tail
5′-HS—C₆-ₛ^{m}CₛTₛ^{m}CₛAₛaₛtₛcₛcₛaₛtₛgₛgₛ^{m}CₛAₛGₛc-3′;

(iv) antisense ErbB3 LNA oligomer modified with a C₆—SH tail
5′-HS—C₆-ₛTₛAₛGₛcₛcₛtₛgₛtₛcₛaₛcₛtₛtₛ^{Me}CₛTₛ^{Me}C-3′

(v) antisense ErbB3 LNA oligomer modified with a C₆—SH tail
5′-HS—C₆-ₛGₛ^{Me}CₛTₛcₛcₛaₛgₛaₛcₛaₛtₛcₛaₛ^{Me}CₛTₛ^{Me}C-3′

(vi) Genasense modified with a hindered ester tail

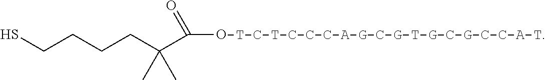

### 7. Targeting Groups

In another aspect, the compounds described herein include a targeting ligand for a specific cell of tissue type. Any known techniques in the art can be used for conjugating a targeting group to the compounds of Formula (I) without undue experimentation.

For example, targeting agents can be attached to the compounds described herein to guide the conjugates to the target area in vivo. The targeted delivery of the compounds described herein enhances the cellular uptake of the compounds described herein, thereby improving the therapeutic efficacies. In certain aspects, some cell penetrating peptides can be replaced with a variety of targeting peptides for targeted delivery to the tumor site.

In one embodiment, the targeting moiety, such as a single chain antibody (SCA) or single-chain antigen-binding antibody, monoclonal antibody, cell adhesion peptides such as RGD peptides and Selectin, cell penetrating peptides (CPPs) such as TAT, Penetratin and (Arg)₉, receptor ligands, targeting carbohydrate molecules or lectins allows the compounds described herein to be specifically directed to targeted regions. See J Pharm Sci. 2006 September; 95(9):1856-72 Cell adhesion molecules for targeted drug delivery, the contents of which are incorporated herein by reference.

Suitable targeting moieties include single-chain antibodies (SCA's) or singe-chain variable fragments of antibodies (sFv). The SCA contains domains of antibodies which can bind or recognize specific molecules of targeting tumor cells.

The terms “single chain antibody” (SCA), “single-chain antigen-binding molecule or antibody” or “single-chain Fv” (sFv) are used interchangeably. The single chain antibody has binding affinity for the antigen. Single chain antibody (SCA) or single-chain Fvs can and have been constructed in several ways. A description of the theory and production of single-chain antigen-binding proteins is found in commonly assigned U.S. patent application Ser. No. 10/915,069 and U.S. Pat. No. 6,824,782, the contents of each of which are incorporated by reference herein.

Typically, SCA or Fv domains can be selected among monoclonal antibodies known by their abbreviations in the literature as 26-10, MOPC 315, 741F8, 520C9, MePC 603, D1.3, murine phOx, human phOx, RFL3.8 sTCR, 1A6, Se155-4,18-2-3,4-4-20,7A4-1, B6.2, CC49,3C2,2c, MA-15C5/K₁₂G_{O}, Ox, etc, (see, Huston, J. S. et al., Proc. Natl. Acad. Sci. USA 85:5879-5883 (1988); Huston, J. S. et al., SIM News 38(4) (Supp):11 (1988); McCartney; J. et al., ICSU Short Reports 10:114 (1990); McCartney, J. E. et al., unpublished results (1990); Nedelman, M. A. et al., J. Nuclear Med. 32 (Supp.):1005 (1991); Huston, J. S. et. al., In: Molecular Design and Modeling: Concepts and Applications, Part B, edited by J. J. Langone, Methods in Enzymology 203:46-88 (1991); Huston, J. S. et al., In: Advances in the Applications of Monoclonal Antibodies in Clinical Oncology, Epenetos, A. A. (Ed.), London, Chapman & Hall (1993); Bird, R. E. et al., Science 242:423-426 (1988); Bedzyk, W. D. et al., J. Biol. Chem. 265:18615-18620 (1990); Colcher, D. et al., J. Nat. Cancer Inst. 82:1191-1197 (1990); Gibbs, R. A. et al., Proc. Natl. Acad. Sci, USA 88:4001-4004 (1991); Milenic, D. E. et al., Cancer Research 51:6363-6371 (1991); Pantoliano, M. W. et al., Biochemistry 30:10117-10125 (1991); Chaudhary, V. K. et al., Nature 339:394-397 (1989); Chaudhary, V. K. et al., Proc. Natl. Acad. Sci. USA 87:1066-1070 (1990); Batra, J. K. et al., Biochem. Biophys. Res. Comm. 171:1-6 (1990); Batra, J. K. et al., J. Biol. Chem. 265:15198-1520 (1990); Chaudhary, V. K. et al., Proc. Natl. Acad Sci. USA 87:9491-9494 (1990); Batra, J. K. et al., Mol. Cell. Biol. 11:2200-2205 (1991); Brinkmann, U. et al., Proc. Natl. Acad. Sci. USA 88:8616-8620 (1991); Seetharam, S. et al., J. Biol. Chem. 266:17376-17381 (1991); Brinkmann, U. et al., Proc. Natl. Acad. Sci. USA 89:3075-3079 (1992); Glockshuber, R. et al., Biochemistry 29:1362-1367 (1990); Skerra, A. et al., Bio/Technol. 9:273-278 (1991); Pack, P, et al., Biochemistry 31:1579-1534 (1992); Clackson, T. et al., Nature 352:624-628 (1991); Marks, J. D. et al., J. Mol. Biol. 222:581-597 (1991); Iverson, B. L. et al., Science 249:659-662 (1990); Roberts, V. A. et al., Proc. Natl. Acad. Sci. USA 87:6654-6658 (1990); Condra, J. H. et al., J. Biol. Chem. 265:2292-2295 (1990); Laroche, Y. et al., J. Biol. Chem. 266:16343-16349 (1991); Holvoet, P. et al., J. Biol. Chem. 266:19717-19724 (1991); Anand, N. N. et al., J. Biol. Chem. 266:21874-21879 (1991); Fuchs, P. et al., Biol Technol. 9:1369-1372 (1991); Breitling, F. et al., Gene 104:104-153 (1991); Seehaus, T. et al., Gene 114:235-237 (1992); Takkinen, K. et al., Protein Engng. 4:837-841 (1991); Dreher, M. L. et al., J. Immunol. Methods 139:197-205 (1991); Mottez, E. et al., Eur. J. Immunol. 21:467-471 (1991); Traunecker, A. et al., Proc. Natl. Acad. Sci. USA 88:8646-8650 (1991); Traunecker, A. et al., EMBO J. 10:3655-3659 (1991); Hoo, W. F. S. et al., Proc. Natl. Acad. Sci. USA 89:4759-4763 (1993)). Each of the forgoing publications is incorporated herein by reference.

A non-limiting list of targeting groups includes vascular endothelial cell growth factor, FGF2, somatostatin and somatostatin analogs, transferrin, melanotropin, ApoE and ApoE peptides, von Willebrand's Factor and von Willebrand's Factor peptides, adenoviral fiber protein and adenoviral fiber protein peptides, PD1 and PD1 peptides, EGF and EGF peptides, RGD peptides, folate, anisamide, etc. Other optional targeting agents appreciated by artisans in the art can be also employed in the compounds described herein.

In one preferred embodiment, the targeting agents useful for the compounds described herein include single chain antibody (SCA), RGD peptides, selectin, TAT, penetratin, (Arg)₉, folic acid, anisamide, etc., and some of the preferred structures of these agents are:

| | | |
|---|---|---|
| C-TAT: | (SEQ ID NO: 17) | CYGRKKRRQRRR; |
| C-(Arg)q: | (SEQ ID NO: 18) | CRRRRRRRRR; |

RGD can be linear or cyclic:

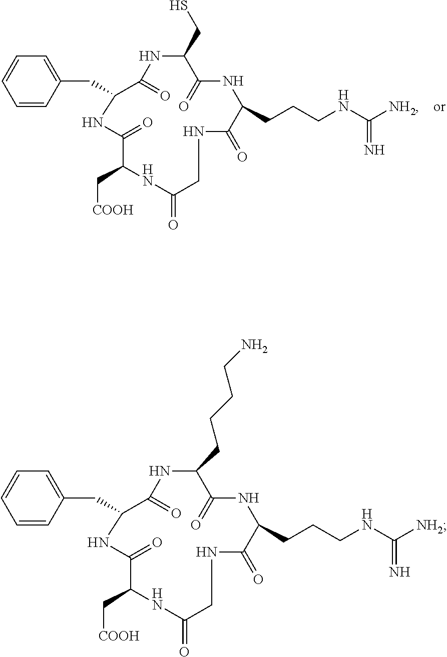

Folic acid is a residue of

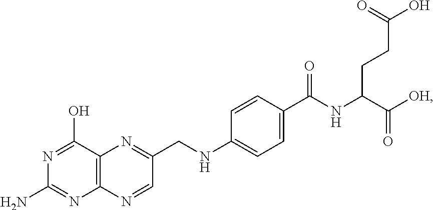

and

Anisamide is p-MeO-Ph-C(═O)OH.

Arg₉ can include a cysteine for conjugating such as CRRRRRRRRR and TAT can add an additional cysteine at the end of the peptide such as CYGRKKRRRC.

For purpose of the current invention, the abbreviations used in the specification and figures represent the following structures:

(i) C-diTAT (SEQ ID NO: 19)=CYGRKKRRQRRRYGRKKRRQRRR-NH₂;

(ii) Linear RGD (SEQ ID NO: 20)=RGDC;

(iii) Cyclic RGD (SEQ ID NO: 21 and SEQ ID NO: 22)=c-RGDFC or c-RGDFK;

(iv) RGD-TAT (SEQ ID NO: 23)=CYGRKKRRQRRRGGGRGDS-NH₂; and

(v) Arg₉ (SEQ ID NO: 24)=RRRRRRRRR.

Alternatively, the targeting group include sugars and carbohydrates such as galactose, galactosamine, and N-acetyl galactosamine; hormones such as estrogen, testosterone, progesterone, glucocortisone, adrenaline, insulin, glucagon, cortisol, vitamin D, thyroid hormone, retinoic acid, and growth hormones; growth factors such as VEGF, EGF, NGF, and PDGF; neurotransmitters such as GABA, Glutamate, acetylcholine, NOGO; inostitol triphosphate; epinephrine; norepinephrine; Nitric Oxide, peptides, vitamins such as folate and pyridoxine, drugs, antibodies and any other molecule that can interact with an cell surface receptor in vivo or in vitro.

### 8. Endosomal Release-Promoting Group

In one aspect of the present invention, the compounds described herein include an endosomal release-promoting moiety/group. The endosomal release-promoting group facilitates release of the biologically active agent into the cytosol after the compounds enter the cells.

The histidine-rich peptide contains about 3 to about 40 amino acids, and preferably from about 3 to about 25 amino acids (e.g., 3, 4, 5, 6, 7, 8, 9, 10, 11, 12, 13, 14, 16, 16, 17, 18, 19, 20, 21, 22, 23, 24, 25). More preferably, the histidine-rich peptide contains a mixture of histidine and lysine. The histidine-rich peptide contains histidines ranging from about 30% to about 100%. (e.g., above about 50%, 70%, 80%, 90% or 100%).

In one preferred embodiment, the endosomal release-promoting moiety includes (His)ₙ, wherein His is a histidine, and (n) is a positive integer, preferably a positive integer equal to or greater than 3, (e.g., a positive integer from about 3 to about 20), and more preferably, a positive integer from about 3 to about 10 (e.g., 3, 4, 5, 6, 7, 8, 9, 10). For example, the endosomal release-promoting moiety is -His-His-His-. In another example, the histidine-rich peptides include, but are not limited to, HHHK (SEQ ID NO: 25), HHHKHHHK (SEQ ID ND: 26), and HHHHHHHH (SEQ ID NO: 27). Without being bound by any theory, once the compounds of Formula (I) are selectively delivered to a target area and enters the cells, the endosomal releasing group is activated in the acidic intracellular endosome environment and promote release of the oligonucleotides.

### 9. Nuclear Localization Signal

Once the oligonucleotide is released in the cytosol, some therapeutic oligonucleotides need to be delivered inside the nucleus in order to express their biological activity. In the present invention, nuclear localization signal peptides can guide the oligonucleotides to the nucleus. Some known nuclear localization signal moieties, such as TAT or CGVKRKKKP (SEQ ID NO: 28), can be employed for this purpose.

Alternatively, the nuclear localization signal peptide is selected from among CGVKRKKKP (SEQ ID NO: 28), CYGRKKRRQRRR (SEQ ID NO: 29), YGRKKRRQRRRC (SEQ ID NO: 30), YGRKKRRQRRR (SEQ ID NO: 31), PKKKRKVEDPYC (SEQ ID NO: 32), VQRKRQKLM (SEQ ID NO: 33), and CGYGPKKKRKVGG (SEQ ID NO: 34).

### 10. Diagnostic Agents

A further aspect of the invention provides the compounds optionally prepared with a diagnostic tag linked to the compounds described herein, wherein the tag is selected for diagnostic or imaging purposes.

The compounds described herein can be labeled or tagged. Suitable labels or tags (the terms are used interchangeably herein) include, e.g., biotinylated compounds, fluorescent compounds, and radiolabelled compounds. A suitable tag is prepared by linking any suitable moiety, e.g., an oligonucleotide residue or an amino acid residue, to any art-standard emitting isotope, radio-opaque label, magnetic resonance label, or other non-radioactive isotopic labels suitable for magnetic resonance imaging, fluorescence-type labels, labels exhibiting visible colors and/or capable of fluorescing under ultraviolet, infrared or electrochemical stimulation, to allow for imaging tumor tissue dulling surgical procedures, and so forth. The diagnostic tag is incorporated into and/or linked to a therapeutic moiety (biologically active agents), allowing for monitoring of the distribution of a therapeutic biologically active material within an animal or human patient.

The inventive tagged conjugates are readily prepared, by art-known methods, with any suitable label, including, e.g., radioisotope labels. Simply by way of example, these include ¹³¹Iodine, ¹²⁵Iodine, ^{99m}Technetium and/or ¹¹¹Indium to produce radioimmunoscintigraphic agents for selective uptake into tumor cells, in vivo. For instance, there are a number of art-known methods of linking peptide to Tc-99m, including, simply by way of example, those shown by U.S. Pat. Nos. 5,328,679; 5,888,474; 5,997,844; and 5,997,845, incorporated by reference herein.

### 11. Non-Antigenic Polymer

A further aspect of the invention provides compounds described herein containing a polymer. Polymers contemplated in the compounds described herein are preferably water soluble and substantially non-antigenic, and include, for example, polyalkylene oxides (PAO's). The compounds described herein further include linear, terminally branched or multi-armed polyalkylene oxides. In one preferred aspect of the invention, the polyalkylene oxide includes polyethylene glycols and polypropylene glycols. More preferably, the polyalkylene oxide includes polyethylene glycol (PEG).

The polyalkylene oxide has a total number average molecular weight of from about 200 to about 100,000 daltons, preferably from about 5,000 to about 60,000 daltons. The polyalkylene oxide can be more preferably from about 5,000 to about 25,000 or yet more preferably from about 20,000 to about 45,000 daltons. In some particularly preferred embodiments, the compounds described herein include the polyalkylene oxide having a total number average molecular weight of from about 30,000 to about 45,000 daltons. In one particular embodiment, polymeric portion has a total number average molecular weight of about 40,000 daltons. Alternatively, the polyalkylene oxide has a number average molecular weight of from about 200 to about 20,000 daltons. The polyalkylene oxide can be more preferably from about 500 to about 10,000, and yet more preferably from about 1,000 to about 5,000 daltons. In one particular embodiment, polymeric portion has a total number average molecular weight of about 2,000 daltons. In one embodiment, the PEG is a polyethylene glycol with a number average molecular weight ranging from about 200 to about 20,000 daltons, from about 500 to about 10,000 daltons, or from about 1,000 to about 5,000 daltons (i.e., about 1,500 to about 3,000 daltons). In one particular embodiment, the PEG has a molecular weight of about 2,000 daltons. In another particular embodiment, the PEG has a molecular weight of about 750 daltons.

PEG is generally represented by the structure:
—O—(CH₂CH₂O)ₓ—

where (x) is a positive integer of from about 5 to about 2300 so that the polymeric portion of the compounds described herein has a number average molecular weight of from about 200 to about 100,000 daltons, (x) represents the degree of polymerization for the polymer, and is dependent on the molecular weight of the polymer.

Alternatively, the polyethylene glycol (PEG) residue portion can be represented by the structure:

—Y₇₁—(CH₂CH₂O)ₓ—CH₂CH₂Y₇₁—,

—Y₇₁—(CH₂CH₂O)ₓ—CH₂C(═Y₇₂)—Y₇₁—,

—Y₇₁—C(═Y₇₂)—(CH₂)ₐ₁₁—Y₇₃—(CH₂CH₂O)ₓ—CH₂CH₂—Y₇₃—(CH₂)ₐ₁₁—C(═Y₇₂)—Y₇₁— and

—Y₇₁—(CR₇₁R₇₂)ₐ₁₁—Y₇₃—(CH₂)_{b11}—O—(CH₂CH₂O)ₓ—(CH₂)_{b11}—Y₇₃—(CR₇₁R₇₂)ₐ₁₁—Y₇₁—,

wherein:

Y₇₁ and Y₇₃ are independently O, S, SO, SO₂, NR₇₃ or a bond;

Y₇₂ is O, S, or NR₇₄;

R₇₁₋₇₄ are independently selected from among hydrogen, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy and substituted arylcarbonyloxy, preferably hydrogen, methyl, ethyl or propyl;

(a11) and (b11) are independently zero or positive integers, preferably zero or positive integers of from about 1 to about 6 (i.e., 1, 2, 3, 4), and more preferably 1; and

(x) is an integer of from about 5 to about 2300, for example, from about 5 to about 460.

The terminal end (A group) of PEG can end with H, NH₂, OH, CO₂H, C₁₋₆ alkyl (e.g., methyl, ethyl, propyl), C₁₋₆ alkoxy (e.g., methoxy, ethoxy, propyloxy), acyl or aryl. In one embodiment, the terminal hydroxyl group of PEG is substituted with a methoxy or methyl group. In one preferred embodiment, the PEG employed in the compounds described herein and/or the PEG lipid is methoxy PEG.

Branched or U-PEG derivatives are described in U.S. Pat. Nos. 5,643,575, 5,919,455, 6,113,906 and 6,566,506, the disclosure of each of which is incorporated herein by reference. A non-limiting list of such polymers corresponds to polymer systems (i)-(vii) with the following structures:

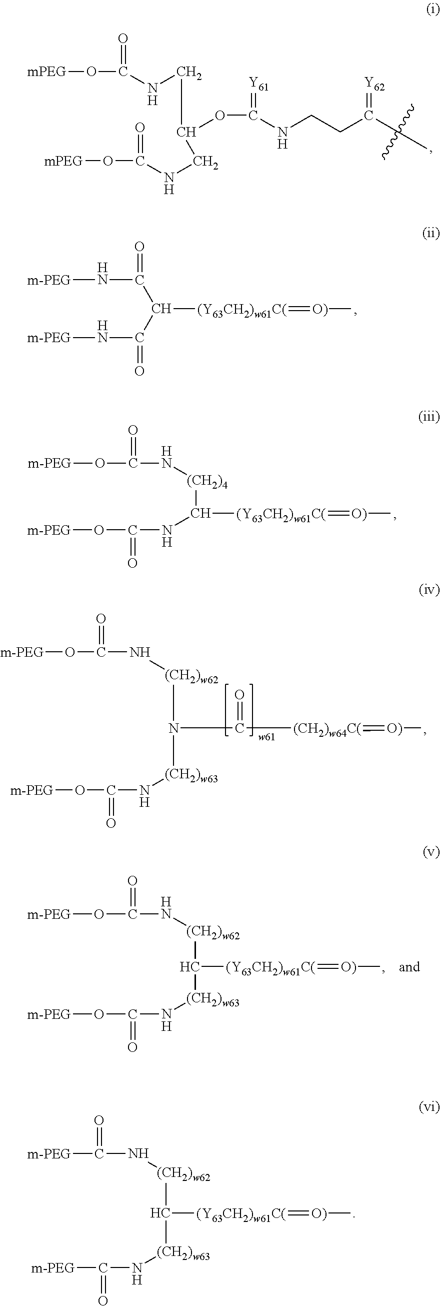

wherein:

Y₆₁₋₆₂ are independently O, S or NR₆₁;

Y₆₃ is O, NR₆₂, S, SO or SO₂

(w62), (w63) and (w64) are independently 0 or positive integers;

(w61) is 0 or 1;

mPEG is methoxy PEG
wherein PEG is previously defined and a total molecular weight of the polymer portion is from about 5,000 to about 100,000 daltons; and

R₆₁ and R₆₂ are independently selected from among hydrogen, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, and substituted and arylcarbonyloxy.

In yet another embodiment, the polymers prior to the conjugation to the compounds described herein include multi-arm PEG-OH or “star-PEG” products such as those described in NOF Corp. Drug Delivery System catalog, Ver. 8, April 2006, the disclosure of which is incorporated herein by reference. The polymers can be converted into suitably activated forms, using the activation techniques described in U.S. Pat. No. 5,122,614 or 5,808,096. Specifically, such PEG can be of the formula:

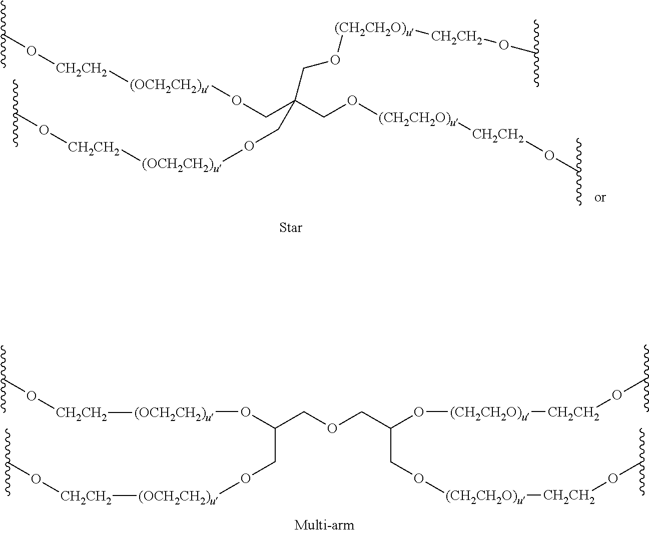

wherein:

(u′) is an integer front about 4 to about 455; and up to 3 terminal portions of the residue is/are capped with a methyl or other lower alkyl.

In one embodiment, the degree of polymerization for the polymer (u′) is from about 28 to about 341 to provide polymers having a total number average molecular weight of from about 5,000 Da to about 60,000 Da, and preferably from about 114 to about 239 to provide polymers having a total number average molecular weight of from about 20,000 Da to about 42,000 Da. (u′) represents the number of repeating units in the polymer chain and is dependent on the molecular weight of the polymer. In one particular embodiment, (u′) is about 227 to provide the polymeric portion having a total number average molecular weight of about 40,000 Da.

In certain embodiments, all four of the PEG arms can be converted to suitable activating groups, for facilitating attachment to other molecules (e.g., oligonucleotides, targeting groups, endosomal release-promoting groups). Such compounds prior to conversion include:

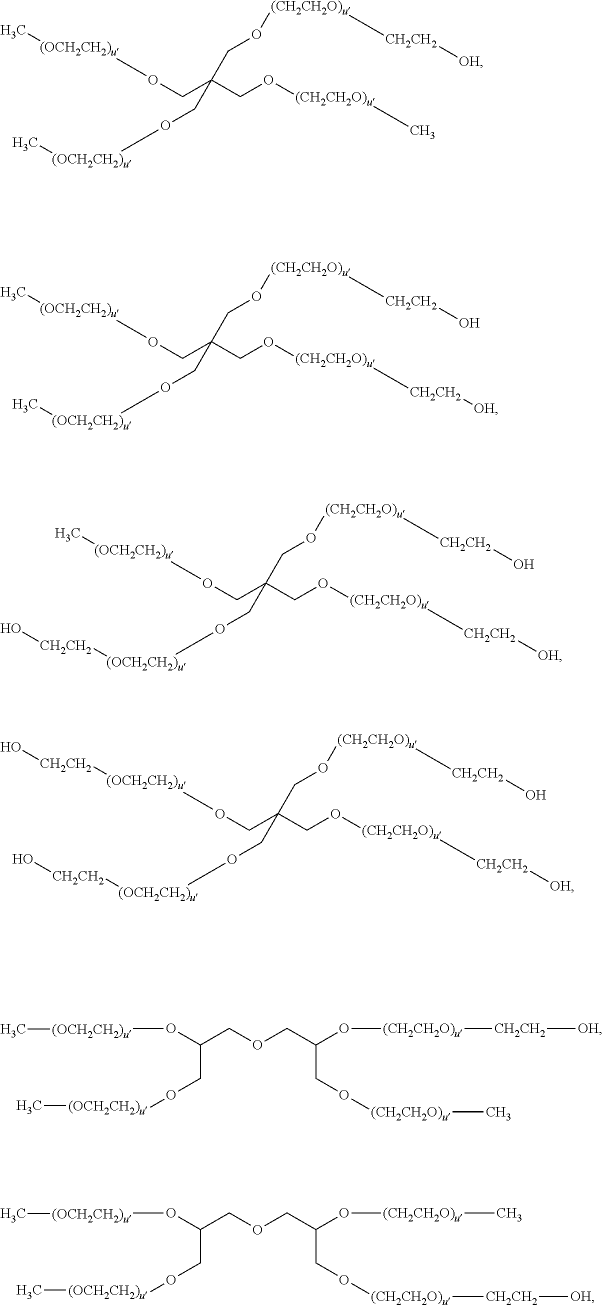

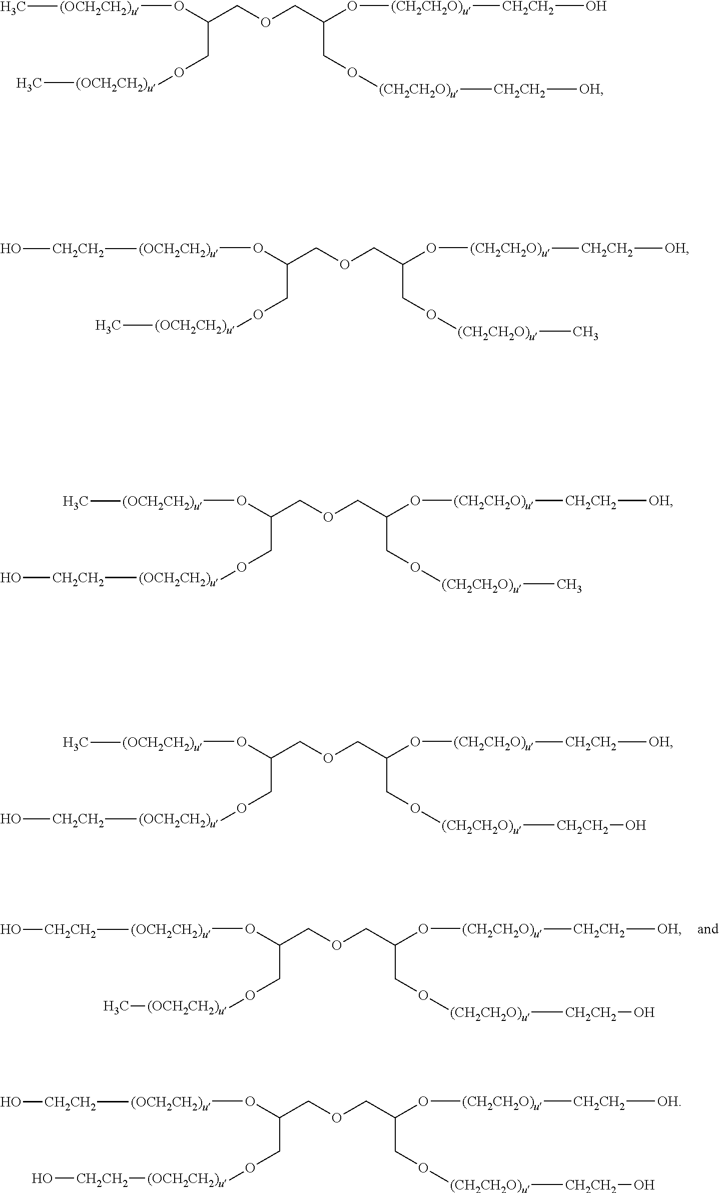

PEG may be conjugated to the compounds described herein directly or via a linker moiety. The polymers for conjugation to a compound of Formula (I) are converted into a suitably activated polymer, using the activation techniques described in U.S. Pat. Nos. 5,122,614 and 5,808,096 and other techniques known in the art without undue experimentation.

Examples of activated PEGs useful for the preparation of a compound of Formula (I) include, for example, methoxypolyethylene glycol-succinate, methoxypolyethylene glycol-succinimidyl succinate (mPEG-NHS), methoxypolyethyleneglycol-acetic acid (mPEG-CH₂COOH), methoxypolyethylene glycol-amine (mPEG-NH₂), and methoxypolyethylene glycol-tresylate (mPEG-TRES).

In certain aspects, polymers having terminal carboxylic acid groups can be employed in the compounds described herein. Methods of preparing polymers having terminal carboxylic acids in high purity are described in U.S. patent application Ser. No. 11/328,662, the contents of which are incorporated herein by reference.

In alternative aspects, polymers having terminal amine groups can be employed to make the compounds described herein. The methods of preparing polymers containing terminal amines in high purity are described in U.S. patent application Ser. Nos. 11/508,507 and 11/537,172, the contents of each of which are incorporated by reference.

In yet a further aspect of the invention, the polymeric substances included herein are preferably water-soluble at room temperature. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained.

In yet a further embodiment and as an alternative to PAO-based polymers such as PEG, one or more effectively non-antigenic materials such as dextran, polyvinyl alcohols, carbohydrate-based polymers, hydroxypropylmethacrylamide (HPMA), polyalkylene oxides, and/or copolymers thereof can be used. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses, such as hydroxymethylcellulose or hydroxyethylcellulose. See also commonly-assigned U.S. Pat. No. 6,153,655, the contents of which are incorporated herein by reference. It will be understood by those of ordinary skill that the same type of activation is employed as described herein as for PAO's such as PEG. Those of ordinary skill in the art will further realize that the foregoing list is merely illustrative and that all polymeric materials having the qualities described herein are contemplated. For purposes of the present invention, “substantially or effectively non-antigenic” means polymeric materials understood in the art as being nontoxic and not eliciting an appreciable immunogenic response in mammals.

### B. Preparation of Compounds of Formula (I)

Synthesis of representative, specific compounds, is set forth in the Examples. Generally, however, the compounds of the present invention can be prepared in several fashions. In one embodiment, the methods of preparing compounds of Formula (I) described herein includes conjugating an endosomal release-promoting group to a targeting group, followed by conjugating the resulting intermediate to a biologically active agent such as oligonucleotides, via an acid-labile linker such as a disulfide bond. In another embodiment, the methods of preparing compounds of Formula (I) described herein include reacting a trifunctional compound having three different activating groups or functional groups with three different molecules such as a cell targeting group, an oligonucleotide, or a nuclear localizing signal peptide.

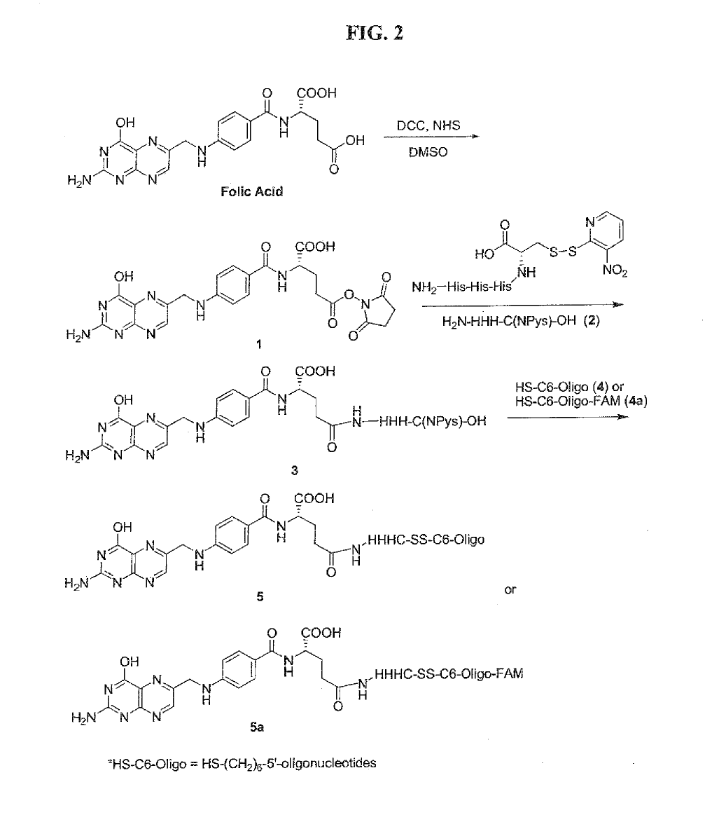

One illustrative example of preparing compounds of Formula (I) is shown in FIG. 2. First, a targeting group such as folic acid is linked to an endosomal release-promoting moiety containing an activated thiol group (i.e., compound 2). The activated thiol group of the resulting intermediate containing a targeting moiety and an endosomal release promoting moiety (i.e., compound 3) is reacted with a thiol group linked to an oligonucleotide (i.e., compound 4) to provide compounds of Formula (I).

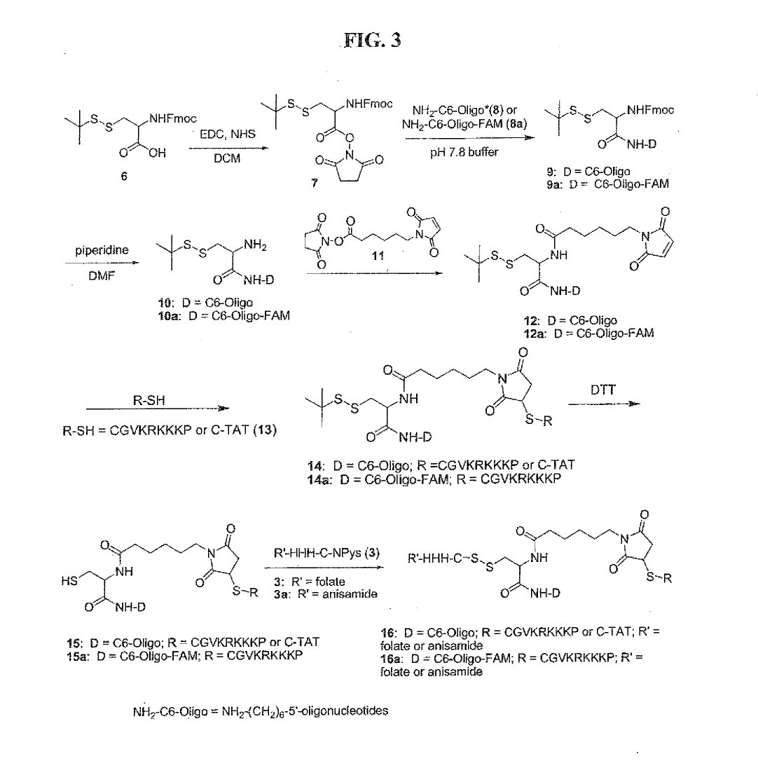

Another illustrative example of preparing compounds of Formula (I) is shown in FIG. 3. A trifunctional compound having three different activating groups and/or functional groups such as NHS, t-butyl thioether as a thiol activating group, and Fmoc as an amine protecting group is prepared. The NHS ester (compound 7) is reacted with a terminal amine of an oligonucleotide to provide an oligonucleotide-containing intermediate (compound 9). The amine protecting group is removed from the intermediate. The unprotected amine group of the intermediate is reacted with a bifunctional spacer containing a maleimide functional group, followed by conjugating to a nuclear localization signaling peptide via the maleimide functional group to provide a compound containing an oligonucleotide and a nuclear localization signaling moiety permanently linked to the trifunctional compound. The thiol protecting group is removed from the compound containing an oligonucleotide and a nuclear localization signaling moiety. The unprotected thiol group is reacted with an endosomal release-promoting moiety via a disulfide bond to provide a compound of Formula (I). Alternatively, the trifunctional compound can be linked to an endosomal release-promoting moiety, an oligonucleotide and a nuclear localization signaling moiety in a different order.

Activation of a carboxylic acid group with NHS (e.g. the preparation of compound 7) can be carried out using standard organic synthetic techniques in the presence of a base, using coupling agents known to those of ordinary skill in the art such as 1,3-diisopropylcarbodiimide (DIPC), dialkyl carbodiimides, 2-halo-1-alkylpyridinium halides, 1-(3-dimethylaminopropyl)-3-ethyl carbodiimide (EDC), propane phosphonic acid cyclic anhydride (PPACA) and phenyl dichlorophosphates.

In a further embodiment, when an activated reagent, such as DSC, PNP carbonate, PNP-chloride, is employed, a coupling agent is not required and the reaction proceeds in the presence of a base.

Generally, the coupling reactions are preferably prepared by reacting an activated compound with an amine containing nucleophile in the presence of a base such as DMAP or DIEA. Preferably, the reaction is carried out in an inert solvent such as methylene chloride, chloroform, toluene, DMF or mixtures thereof. The reaction is also preferably conducted in the presence of a base, such as DMAP, DIEA, pyridine, triethylamine, etc. at a temperature from −4° C. to about 70° C. (e.g. −4° C. to about 50° C.). In one preferred embodiment, the reaction is performed at a temperature from 0° C. to about 25° C. or 0° C. to about room temperature.

Removal of a protecting group, such as Fmoc, from an amine-compounding compound, can be carried out with a base, such as piperidine, DMAP. On the other hand, a protecting group, such as BOC, can be removed with a strong acid such as trifluoroacetic acid (TFA), HCl, sulfuric acid, etc., or catalytic hydrogenation, radical reaction, etc. In one embodiment, deprotection of Fmoc group is carried out with piperidine. The deprotection reaction can be carried out at a temperature from −4° C. to about 5° C. Preferably, the reaction is carried out at a temperature from 0° C. to about 25° C. or to room temperature. In another embodiment, the deprotection of Fmoc group is carried out at room temperature.

Coupling agents known to those of ordinary skill in the art, such as 1,3-diisopropylcarbodiimide (DIPC), dialkyl carbodiimides, 2-halo-1-alkylpyridinium halides, 1-(3-dimethylaminopropyl)-3-ethyl carbodiimide (EDC), propane phosphonic acid cyclic anhydride (PPACA) and phenyl dichlorophosphates, can be employed in the preparation of compounds described herein. The reaction preferably is conducted in the presence of a base, such as DMAP, DIEA, pyridine, triethylamine, etc. at a temperature from −4° C. to about 50° C. In one embodiment, the reaction is performed at a temperature from 0° C. to about 25° C. or to room temperature.

Conjugation of a thiol containing moiety to form a labile disulfide bond is carried out employing an activated thiol such as NPys in compound 2. The disulfide bond provides releasable connection between two groups, such that the bond degrades in an acidic environment to release oligonucleotides optionally conjugated with nuclear localization signaling peptides.

Alternatively, conjugation of a thiol containing moiety is carried out using a function group such as maleimide, as described in FIG. 3 to form a thio ether bond which is stable to hydrolysis. This conjugation reaction between a thiol containing moiety and maleimide provides a permanent attachment between two reacting groups.

### C. Compounds of Formula (I)

Some particular embodiments prepared by the methods described herein have the structure:

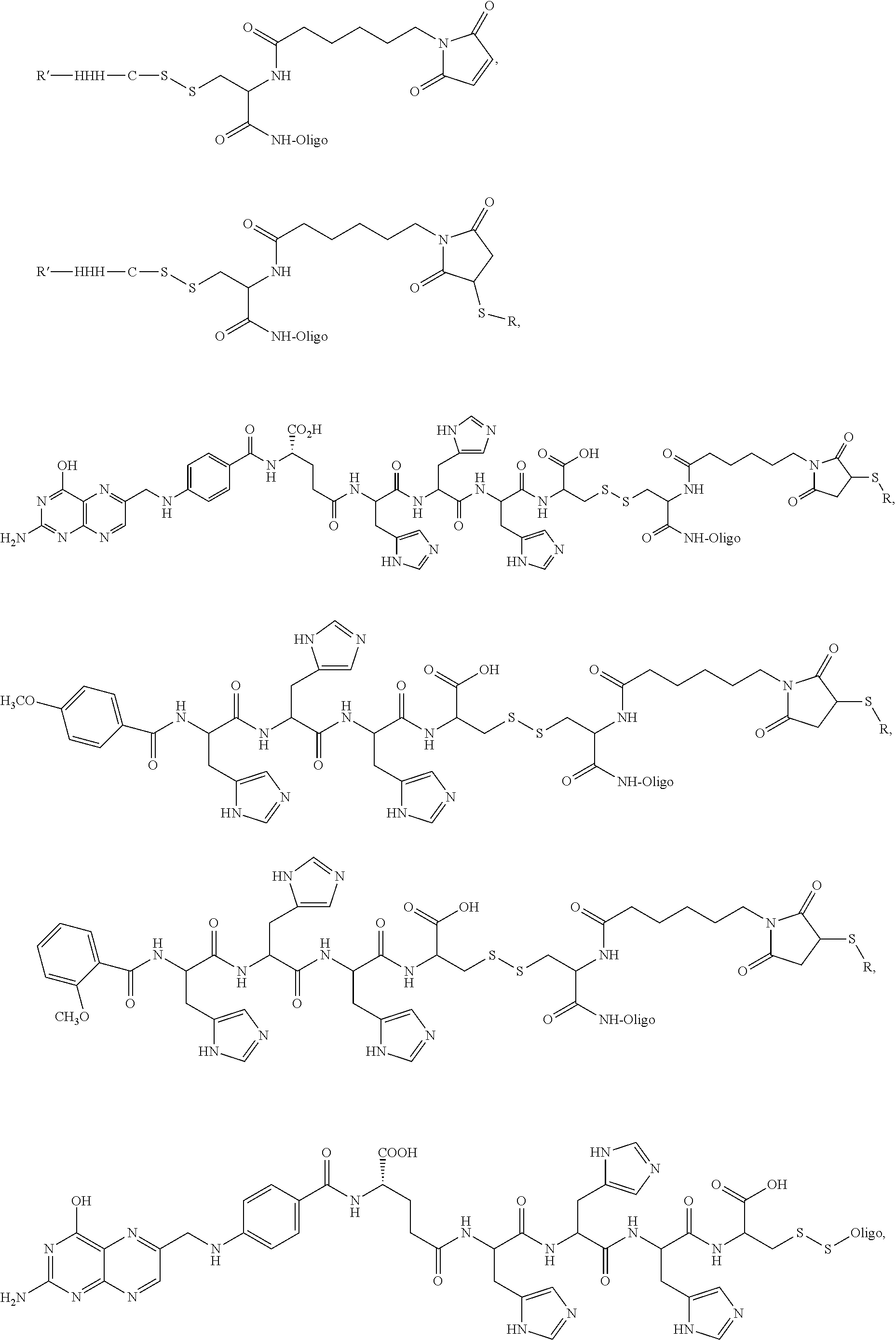

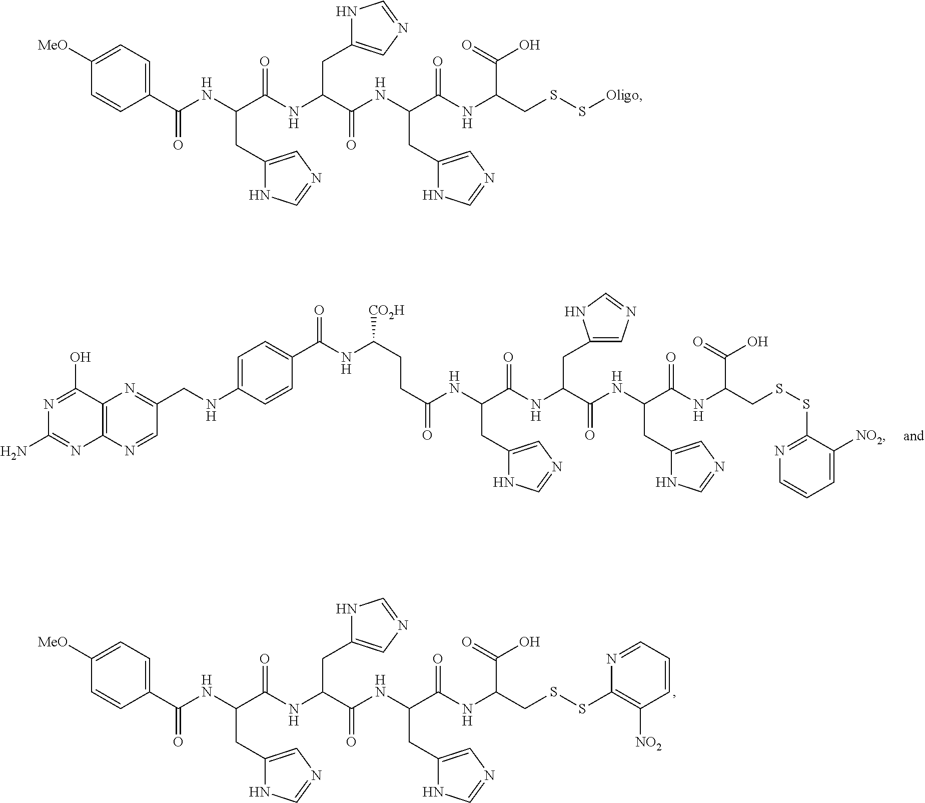

wherein

Oligo is an oligonucleotide such as oligonucleotides modified with C₁₋₆ alkyl (i.e., -5′-(CH₂)₆-antisense-Survivin LNA oligomer, -5′-(CH₂)₆-antisence-EtbB3 LNA oligomer, and -5′-(CH₂)₆-antisense-HIF-1α LNA oligomer);

R′ is a targeting group such as folate and anisamide; and

R is a nuclear localization signal peptide.

### D. Nanoparticle

In a further aspect of the invention, the compounds of Formula (I) are included in a nanoparticle composition. In accordance with this aspect of the invention, the nanoparticle composition for the delivery of nucleic acids (i.e., an oligonucleotide) may include a cationic lipid, a fusogenic lipid and a PEG lipid.

In one embodiment, the nanoparticle composition further includes cholesterol.

In one aspect, the nanoparticle composition contains a cationic lipid in a molar ratio ranging from about 10% to about 99.9% of the total lipid/pharmaceutical carrier present in the nanoparticle composition.

The cationic lipid component can range from about 2% to about 60%, from about 5% to about 50%, from about 10% to about 45%, from about 15% to about 25%, or from about 30% to about 40% of the total lipid present in the nanoparticle composition.

In one preferred embodiment, the cationic lipid is present in amounts from about 15 to about 25% (i.e., 15, 17, 18, 20 or 25%) of total lipid present in the nanoparticle composition.

In another preferred aspect of the nanoparticle composition described herein, the compositions contain a total fusogenic/non-cationic lipid, including cholesterol and/or noncholesterol-based fusogenic lipid, in a molar ratio of from about 20% to about 85%, from about 25% to about 85%, from about 60% to about 80% (e.g., 65, 75, 78, or 80%) of the total lipid present in the nanoparticle composition. In one embodiment, a total fusogenic/non-cationic lipid is about 80% of the total lipid present in the nanoparticle composition.

In one preferred embodiment, a noncholesterol-based fusogenic/non-cationic lipid is present in a molar ratio of from about 25 to about 78% (25, 35, 47, 60, or 78%), or from about 60 to about 78% of the total lipid present in the nanoparticle composition. In one embodiment, a noncholesterol-based fusogenic/non-cationic lipid is about 60% of the total lipid present in the nanoparticle composition.

In a further preferred aspect, the nanoparticle composition includes cholesterol in addition to non-cholesterol fusogenic lipid, in a molar ratio ranging from about 0% to about 60% from about 10% to about 60%, or from about 20% to about 50% (e.g., 20, 30, 40 or 50%) of the total lipid present in the nanoparticle composition. In one embodiment, cholesterol is about 20% of the total lipid present in the nanoparticle composition.

In another aspect of the invention, the PEG-lipid contained in the nanoparticle composition ranges in a molar ratio of from about 0.5% to about 20% and from about 1.5% to about 18% of the total lipid present in the nanoparticle composition. In one preferred embodiment of the nanoparticle composition, the PEG lipid is included in a molar ratio of from about 2% to about 10% (e.g., 2, 3, 4, 5, 6, 7, 8, 9 or 10%) of the total lipid. In one embodiment, a total PEG lipid is about 2% of the total lipid present in the nanoparticle composition.

In one particular embodiment, a nanoparticle composition includes the cationic lipid having the structure:

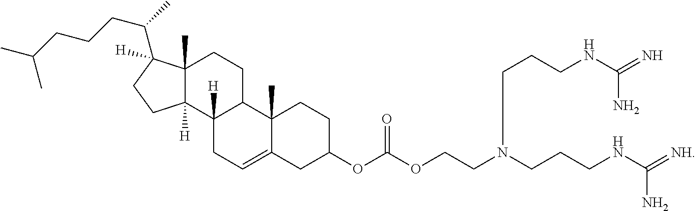

Details of cationic lipids, fusogenic lipids and PEG lipids, and methods of preparing nanoparticles are described in PCT/US09/52396 and U.S. Provisional Application No. 61/085,289 entitled “Nanoparticle Compositions For Nucleic Acids Delivery System”, the contents of bath of which are incorporated herein by reference.

In yet a further embodiment, the nanoparticle composition contains releasable fusogenic lipids based on an acid-labile imine linker and a zwitterion-containing moeity. Such releasable fusogenic lipids allow nucleic acids (oligonucleotides) to dissociate from the delivery system such as nanoparticles after the delivery system enters the cells. Additional details of such releasable fusogenic lipids are also described in U.S. Provisional Patent Application No. 61/115,378, entitled “Releasable Fusogenic Lipids Based on Zwitterionic Moiety For Nucleic Acids Delivery System”, the contents of which axe incorporated herein by reference.

In yet a further embodiment, PEG lipids can include a releasable linker such as ketal or imine. Such releasable PEG lipids allow nucleic acids (oligonucleotides) to dissociate from the delivery system such as nanoparticles after the delivery system enters the cells. Additional details of such releasable PEG lipids are also described in U.S. Provisional Patent Application Nos. 61/115,379 and 61/115,371, entitled “Releasable Polymeric Lipids Based on Imine Moiety For Nucleic Acids Delivery System” and “Releasable Polymeric Lipids Based on Ketal or Acetal Moiety For Nucleic Acids Delivery System” respectively, and PCT Patent Application No. ______, filed on even date, and entitled “Releasable Polymeric Lipids For Nucleic Acids Delivery Systems”, the contents of which are incorporated herein by reference.

### E. Methods of Treatment

The compounds described herein or nanoparticles encapsulating the compounds described herein can be employed in the treatment for preventing, inhibiting, reducing or treating any trait, disease or condition that is related to or responds to the levels of target gene expression in a cell or tissue, alone or in combination with other therapies.

One aspect of the present invention provides methods of introducing or delivering therapeutic agents such as nucleic acids/oligonucleotides into a mammalian cell in vivo and/or in vitro.

The method according to the present invention includes contacting a cell with the compounds described herein. The delivery can be made in vivo as part of a suitable pharmaceutical composition or directly to the cells in an ex vivo environment.

The present invention is useful for introducing oligonucleotides to a mammal. The compounds described herein can be administered to a mammal, preferably human.

According to the present invention, the present invention preferably provides methods of inhibiting, downregulating, or modulating a gene expression in mammalian cells or tissues. The downregulation or inhibition of gene expression can be achieved in vivo and/or in vitro. The methods include contacting human cells or tissues with the compounds described herein or nanoparticles encapsulating the compounds described herein. Once the contacting has occurred, successful inhibition or down-regulation of gene expression such as in mRNA, protein levels or protein activities shall be deemed to occur when at least about 10%, preferably at least about 20% or higher (e.g., 30%, 40%, 50%, 60%) is realized in vivo or in vitro when compared to that observed in the absence of the compounds described herein.

For purposes of the present invention, “inhibiting” or “down-regulating” shall be understood to mean that the expression of a target gene, or level of RNAs or equivalent RNAs encoding one or more protein subunits, or activity of one or more protein subunits, such as ErbB3, HIF-1α, Survivin and BCL2, is reduced below that observed in the absence of the compounds described herein.

In one preferred embodiment, target genes include, for example, but are not limited to, oncogenes, pro-angiogenesis pathway genes, pro-cell proliferation pathway genes, viral infectious agent genes, and pro-inflammatory pathway genes.

Preferably, gene expression of a target gene is inhibited in cancer cells or tissues, for example, brain, breast, colorectal, gastric, lung, mouth, pancreatic, prostate, skin or cervical cancer cells. The cancer cells or tissues can be from one or more of the following: solid tumors, lymphomas, small cell lung cancer, acute lymphocytic leukemia (ALL), pancreatic cancer, glioblastoma, ovarian cancer, gastric cancer, breast cancer, colorectal cancel, prostate cancer, cervical cancer, ovarian cancer and brain tumors, etc.

In one particular embodiment, the compounds according to the methods described herein includes, for example, antisense bcl-2 oligonucleotides, antisense HIF-1α oligonucleotides, antisense survivin oligonucleotides, antisense ErbB3 oligonucleotides, antisense PIK3CA oligonucleotides, antisense HSP27 oligonucleotides, antisense androgen receptor oligonucleotides, antisense Gli2 oligonucleotides, and antisense beta-catenin oligonucleotides.

In one particular treatment, the compounds including oligonucleotides (SEQ ID NO: 1, SEQ ID NOs 2 and 3, SEQ ID NO:3, SEQ ID NO: 4, SEQ ID NO: 5, SEQ ID NO: 6, SEQ ID NO: 7, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, and SEQ ID NO: 16 in which each nucleic acid is a naturally occurring or modified nucleic acid) can be used. The therapy contemplated herein uses nucleic acids encapsulated in the aforementioned nanoparticle. In one embodiment, therapeutic nucleotides containing eight or more consecutive antisense nucleotides can be employed in the treatment.

Alternatively, there are also provided methods of treating a mammal or mammals, including humans. The methods include administering an effective amount of a pharmaceutical composition containing a compound described herein to a mammal, e.g., a patient in need thereof. The efficacy of the methods would depend upon efficacy of the therapeutic agent (e.g., nucleic acids) for the condition being treated.

One aspect of the present invention provides methods of treating various medical conditions in mammals. The methods include administering, to the mammal in need of such treatment, an effective amount of a compound described herein containing a therapeutic agent (nucleic acids). The compounds described herein are useful for, among other things, treating diseases for example, but not limited to, cancer, inflammatory disease, and autoimmune disease.

In one aspect, there are also provided methods of treating a patient having a malignancy or cancer, comprising administering an effective amount of a pharmaceutical composition containing the compound described herein to a patient in need thereof. The cancer being treated can be one or more of the following: solid tumors, lymphomas, small cell lung cancer, acute lymphocytic leukemia (ALL), pancreatic cancer, glioblastoma, ovarian cancer, gastric cancers, colorectal cancer, prostate cancer, cervical cancer, etc. The compounds described herein are useful for treating neoplastic disease, reducing tumor burden, preventing metastasis of neoplasms and preventing recurrences of tumor/neoplastic growths in mammals by downregulating gene expression of a target gene.

In yet another aspect, the present invention provides methods of the growth or proliferation of cancer cells in vivo or in vitro. The methods include contacting cancer cells with the compound described herein. In one embodiment, the present invention provides methods of inhibiting the growth of cancer in vivo or in vitro wherein the cells express ErbB3 gene.

In another aspect, the present invention provides a means to deliver nucleic acids (e.g., antisense ErbB3 LNA oligonucleotides) inside a cancer cell where it can bind to ErbB3 mRNA, e.g., in the nucleus. As a consequence, the ErbB3 protein expression is inhibited, which inhibits the growth of the cancer cells. The methods introduce oligonucleotides (e.g. antisense oligonucleotides including LNA) to cancer cells and reduce target gene (e.g., survivin, HIF-1α or ErbB3) expression in the cancer cells or tissues.

Alternatively, the present provides methods of modulating apoptosis in cancer cells. In yet another aspect, there are also provided methods of increasing the sensitivity of cancer cells or tissues to chemotherapeutic agents in vivo or in vitro.

In yet another aspect, there are provided methods of killing tumor cells in vivo or in vitro. The methods include introducing the compounds described herein to tumor cells to reduce gene expression such as ErbB3 gene and contacting the tumor cells with an amount of at least one anticancer agent (e.g., a chemotherapeutic agent) sufficient to kill a portion of the tumor cells. Thus, the portion of tumor cells killed can be greater than the portion which would have been killed by the same amount of the chemotherapeutic agent in the absence of the compounds described herein.

In a further aspect of the invention, an anticancer/chemotherapeutic agent can be used in combination, simultaneously or sequentially, with the compounds described herein. The compounds described herein can be administered prior to, or concurrently with, the anticancer agent, or after the administration of the anticancer agent. Thus, the compounds described herein can be administered prior to, during, or after treatment of the chemotherapeutic agent.

Still further aspects include combining the therapy employing the compounds described herein with other anticancer therapies for synergistic or additive benefit.

The compounds described herein can be used to deliver a pharmaceutically active agent, preferably having a negative charge or a neutral charge. The pharmaceutically active agents include small molecular weight molecules. Typically, the pharmaceutically active agents have a molecular weight of less than about 1,500 daltons.

In a further embodiment, the compounds described herein can be used to deliver nucleic acids, a pharmaceutically active agent, or in combination thereof.

In yet a further embodiment, the nanoparticle associated with the treatment can contain a mixture of one or more therapeutic nucleic acids (either the same or different, for example, the same or different oligonucleotides), and/or one or more pharmaceutically active agents for synergistic application.

### F. Pharmaceutical Compositions/Formulations

Pharmaceutical compositions/formulations including the compounds described herein or nanoparticles encapsulating the compounds described herein may be for in conjunction with one or more physiologically acceptable carriers comprising excipients and auxiliaries which facilitate processing of the active compounds into preparations which can be used pharmaceutically. Proper formulation is dependent upon the route of administration chosen, i.e. whether local or systemic treatment is treated.

Suitable forms, in part, depend upon the use or the route of entry, for example oral, transdermal, or injection. Factors for considerations known in the art include such as toxicity and any disadvantageous forms that prevent the composition or formulation from exerting its effect.

Administration of pharmaceutical compositions of compounds described herein may be oral, pulmonary, topical or parentarel. Topical administration includes, without limitation, administration via the epidermal, transdermal, ophthalmic routes, including via mucous membranes, e.g., including vaginal and rectal delivery. Parenteral administration, including intravenous, intraarterial, subcutaneous, intraperitoneal or intramuscular injection or infusion, is also contemplated.

In one preferred embodiment, the compounds containing therapeutic oligonucleotides are administered intravenously (i.v.) or intraperitoneally (i.p.). Parenteral routes are preferred in many aspects of the invention.

For injection, including, without limitation, intravenous, intramuscular and subcutaneous injection, the compounds of the invention may be formulated in aqueous solutions, preferably in physiologically compatible buffers such as physiological saline buffer or polar solvents including, without limitation, a pyrrolidone or dimethylsulfoxide.

The compounds may also be formulated for bolus injection or for continuous infusion. Formulations for injection may be presented in unit dosage form, e.g., in ampoules or in multi-dose containers. Useful compositions include, without limitation, suspensions, solutions or emulsions in oily or aqueous vehicles, and may contain adjuncts such as suspending, stabilizing and/or dispersing agents. Pharmaceutical compositions for parenteral administration include aqueous solutions of a water soluble form. Aqueous injection suspensions may contain substances that modulate the viscosity of the suspension, such as sodium carboxymethyl cellulose, sorbitol, or dextran. Optionally, the suspension may also contain suitable stabilizers and/or agents that increase the concentration of the compounds described herein in the solution. Alternatively, the compounds described herein may be in powder form for constitution with a suitable vehicle, e.g., sterile, pyrogen-free water, before use.

For oral administration, the compounds described herein can be formulated by combining the compounds with pharmaceutically acceptable carriers well-known in the art. Such carriers enable the compounds of the invention to be formulated as tablets, pills, lozenges, dragees, capsules, liquids, gels, syrups, pastes, slurries, solutions, suspensions, concentrated solutions and suspensions for diluting in the drinking water of a patient, premixes for dilution in the feed of a patient, and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropyl-methylcelluose, sodium carboxy-methylcellulose, and/or polyvinylpyrrolidone (PVP). If desired, disintegrating agents may be added, such as cross-linked polyvinyl pyrrolidone, agar, or alginic acid. A salt such as sodium alginate may also be used.

For administration by inhalation, the compounds of the present invention can conveniently be delivered in the form of an aerosol spray using a pressurized pack or a nebulizer and a suitable propellant.

The compounds may also be formulated in rectal compositions such as suppositories or retention enemas, using, e.g., conventional suppository bases such as cocoa butter or other glycerides.

In addition to the formulations described previously, the compounds may also be formulated as depot preparations. Such long acting formulations may be administered by implantation (for example, subcutaneously or intramuscularly) or by intramuscular injection. A compound of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

Additionally, the compounds of the present invention may be delivered using a sustained release system, such as semi-permeable matrices of solid hydrophobic polymers containing the compounds. Various sustained-release materials have been established and are well known by those skilled in the art.

In addition, antioxidants and suspending agents can be used in the pharmaceutical compositions of the compounds described herein.

### G. Dosages

Determination of a therapeutically effective amount is well within the capability of those skilled in the art, especially in light of the disclosure herein.

For any conjugate used in the methods of the invention, the therapeutically effective amount can be estimated initially froth in vitro assays. Then, the dosage can be formulated for use in animal models so as to achieve a circulating concentration range that includes the effective dosage. Such information can then be used to more accurately determine dosages useful in patients.

The amount of the pharmaceutical composition that is administered will depend upon the potency of the therapeutic agents conjugated. Generally, the amount of the compounds used in the treatment methods is that amount which effectively achieves the desired therapeutic result in mammals. Naturally, the dosages of the various compounds will vary somewhat depending upon the therapeutic agent conjugated thereto (e.g., oligonucleotides). In addition, the dosage, of course, can vary depending upon the dosage form and route of administration. In general, however, the therapeutic agent (e.g. oligonucleotides) conjugated to the compounds described herein can be administered in amounts ranging from about 0.1 mg/kg/week to about 1 g/kg/week, preferably from about 1 to about 500 mg/kg and more preferably from 1 to about 100 mg/kg (i.e., from about 10 to about 90 mg/kg/week). The range set forth above is illustrative and those skilled in the art will determine the optimal dosing based on clinical experience and the treatment indication. Moreover, the exact formulation, route of administration and dosage can be selected by the individual physician in view of the patient's condition. Additionally, toxicity and therapeutic efficacy of the compounds described herein can be determined by standard pharmaceutical procedures in cell cultures or experimental animals using methods well-known in the art.

Alternatively, an amount of from about 1 mg to about 100 mg/kg/dose (0.1 to 100 mg/kg/dose) can be used in the treatment depending on potency of the nucleic acids. Dosage unit forms generally range from about 1 mg to about 60 mg of an active agent, oligonucleotides.

In one embodiment, the treatment of the present invention includes administering the oligonucleotide conjugated to the compounds described herein in an amount of from about 1 to about 60 mg/kg dose (from about 25 to 60 mg/kg/dose, from about 3 to about 20 mg/kg/dose), such as 60, 45, 35, 30, 25, 15, 5 or 3 mg/kg/dose (either in a single or multiple dose regime) to a mammal. For example, the compounds described herein can be administered introvenously in an amount of 30 or 60 mg/kg/dose at q3d×9.

Alternatively the delivery of the oligonucleotide conjugated to the compounds described herein includes contacting a concentration of oligonucleotides of from about 0.1 to about 1000 μM, preferably, from about 5 to about 1500 μM (i.e. from about 10 to about 1000 μM, from about 30 to about 1000 μM) with tumor cells or tissues in vivo or in vitro.

The compositions may be administered once daily or divided into multiple doses (e.g., q3d) which can be given as part of a multi-week treatment protocol. The precise dose will depend on the stage and severity of the condition, the susceptibility of the tumor to the nucleic acids, and the individual characteristics of the patient being treated, as will be appreciated by one of ordinary skill in the art.

In all aspects of the invention where compounds of the present invention are administered, the dosage amount mentioned is based on the amount of therapeutic agents such as oligonucleotide molecules rather than the amount of conjugates administered.

It is contemplated that the treatment will be given for one or more days until the desired clinical result is obtained. The exact amount, frequency and period of administration of the compound of the present invention will vary, of course, depending upon the sex, age and medical condition of the patent as well as the severity of the disease as determined by the attending clinician.

Still further aspects include combining the compound of the present invention described herein with other anticancer therapies for synergistic or additive benefit.

### EXAMPLES

The following examples serve to provide further appreciation of the invention but are not meant in any way to restrict the effective scope of the invention.

In the examples, all synthesis reactions are run under an atmosphere of dry nitrogen or argon. N-(3-aminopropyl)-1,3-propanediamine), BOC—ON, LiOCl₄, Cholesterol and 1H-Pyrazole-1-carboxamidine-HCl were purchased from Aldrich. All other reagents and solvents were used without further purification. An LNA Oligo-1 targeting survivin gene, Oligo-2 targeting ErbB3 gene and Oligo-3 (scrambled Oligo-2) were prepared in house and their sequences are given in Table 2. The internucleoside linkage is phosphorothioate, ^{m}C represents methylated cytosine, and the upper case letters indicate LNA.

TABLE 2

| LNA Oligo | Sequence |
|---|---|
| Oligo-1 (SEQ ID NO: 1) | 5′-^{m}CT^{m}CAatccatgg^{m}CAGc-3′ |
| Oligo-2 (SEQ ID NO: 6) | 5′-TAGcctgtcactt^{m}CT^{m}C-3′ |
| Oligo-3 (SEQ ID NO: 35) | 5′-TAGcttgtcccat^{m}CT^{m}C-3 |

Following abbreviations are used throughout the examples such as, LNA (Locked nucleic acid), BACC (2-[N,N′-di(2-guanidiniumpropyl)]aminoethyl-cholesteryl-carbonate), Chol (cholesterol), DIEA (diisopropylethylamine), DMAP (4-N,N-dimethylamino-pyridine), DOPE (L-α-dioleoyl phosphatidylethanolamine, Avanti Polar Lipids, USA or NOF, Japan), DLS (Dynamic Light Scattering), DSPC (1,2-distearoyl-sn-glycero-3-phosphocholine) (NOF, Japan), DSPE-PEG (1,2-distearoyl-sn-glycero-3-phosphoethanolamine-N-(polyethylene glycol) 2000 ammonium salt or sodium salt, Avanti Polar Lipids, USA and NOF, Japan), DTT (1,4-dithiothreitol), KD (knockdown), EPC (egg phosphatidylcholine, Avanti Polar Lipids, USA) and C16mPEG-Ceramide (N-palmitoyl-sphingosine-1-succinyl(methoxypolyethylene glycol) 2000, Avanti Polar Lipids, USA). Other abbreviations such as the FAM (6-carboxyfluorescein), FBS (fetal bovine serum), GAPDH (glyceraldehyde-3-phosphate dehydrogenase), DMEM (Dulbecco's Modified Eagle's Medium), MEM (Modified Eagle's Medium), TEAA (tetraethylammonium acetate), TFA (trifluoroacetic acid), RT-qPCR (reverse transcription-quantitative polymerase chain reaction) were also used.

### Example 1

### General NMR Method

¹H NMR spectra were obtained at 300 MHz and ¹³C NMR spectra at 75.46 MHz using a Varian Mercury 300 NMR spectrometer and deuterated chloroform as the solvents unless otherwise specified. Chemical shifts (δ) are reported in parts per million (ppm) downfield from tetramethylsilane (TMS).

### Example 2

### General HPLC Method

The reaction mixtures and the purity of intermediates and final products are monitored by Beckman Coulter System Gold® HPLC instrument. It employs a ZORBAX® 300SB C8 reversed phase column (150×4.6 mm) or a Phenomenex Jupiter® 300A C18 reversed phase column (150×4.6 mm) with a 168 Diode Array UV Detector, using a gradient of 10-90% of acetonitrile in 0.05% TFA at a flow rate of 1 mL/minute or a gradient of 25-35% acetonitrile in 50 mM TEAA buffer at a flow rate of 1 mL/minute. The anion exchange chromatography was run on AKTA explorer 100A from GE healthcare (Amersham Biosciences) using Poros 50HQ strong anion exchange resin from Applied Biosystems packed in an AP-Empty glass column from Waters. Desalting was achieved by using HiPrep 26/10 desalting columns from Amersham Biosciences. (for PEG-Oligo)

### Example 3

### General mRNA Down-Regulation Procedure

The cells were maintained in complete medium (F-12K or DMEM, supplemented with 10% FBS). A 12 well plate containing 2.5×10⁵ cells in each well was incubated overnight at 37° C. Cells were washed once with Opti-MEM® and 400 μL of Opti-MEM® was added per each well. Then, a solution of nanoparticles or Lipofectamine2000® containing oligonucleotides was added to each well. The cells were incubated for 4 hours, followed by addition of 600 μL of media per well, and incubation for 24 hours. After 24 hours of treatment, the intracellular mRNA levels of the target gene, such as human ErbB3, and a housekeeping gene, such as GAPDH were quantified by RT-qPCR. The expression levels of mRNA were normalized.

### Example 4

### General RNA Preparation Procedure

For in vitro mRNA down-regulation studies, total RNA was prepared using RNAqueous Kit® (Ambion) following the manufacturer's instruction. The RNA concentrations were determined by OD_{260 nm} using Nanodrop.

### Example 5

### General RT-qPCR Procedure

All the reagents were from Applied Biosystems: High Capacity cDNA Reverse Transcription Kit® (4368813), 20×PCR master mix (4304437), and TaqMan® Gene Expression Assays kits for human GAPDH (Cat. @0612177) and survivin (BIRK5 Hs00153353). 2.0 μg of total RNA was used for cDNA synthesis in a final volume of 50 μL. The reaction was conducted in a PCR thermocycler at 25° C. for 10 minutes, 37° C. for 120 minutes, 85° C. for 5 seconds and then stored at 4° C. Real-time PCR was conducted with the program of 50° C.-2 minutes, 95° C.-10 minutes, and 95° C.-15 seconds/60° C.-1 minute for 40 cycles. For each qPCR reaction, 1 μL of cDNA was used in a final volume of 30 μL.

### Example 6

### Preparation of Compound 1 (Folate-NHS)

Folic acid (250 mg, 0.566 mmol) was dissolved in DMSO and NHS (110.5 mg, 0.956 mmol), TEA (118 μL, 0.956 mmol), and DCC (137.5 mg, 0.666 mmol) were added. The reaction mixture was stirring at room temperature for overnight. The reaction mixture was filtered and the resulting activated folate-NHS in DMSO was used directly.

### Example 7

### Preparation of Compound 3

A histidine-rich peptide (compound 2, 50 mg, 0.0728 mmol) was dissolved in 1 mL of DMF followed by adding DIEA (26 μL, 0.149 mmol), and 3 mL of Folate-NHS (compound 1, 250 mg, 0.193 mmol) solution it DMSO. The reaction mixture was stirred at room temperature for overnight. The mixture was purified on C18 prep to isolate the product. Molecular weight was confirmed by LC-MS.

### Example 8

### Preparation of Compound 3a

Instead of folic acid, p-Methoxybenzoic acid is treated with the reaction conditions described in Examples 6 and 7 to provide p-methoxybenzoic acid NHS ester.

### Example 9

### Preparation of Compound 5

Compound 3 and HS—C6-Oligo2 (compound 4, 7 mg: HS—C6-antisense ErbB3 oligonucleotide) are dissolved in 2 mL of pH 6.5 phosphate buffer (100 mM). The reaction mixture is purified on HiPrep column with water after 4 hour to isolate the product. LC-MS confirms the molecular weight.

### Example 10

### Preparation of Compound 5a

Compound 3 and HS—C6-Oligo2-FAM (compound 4a, 7 mg) were dissolved in 2 mL of pH 6.5 phosphate buffer (100 mM). The reaction mixture was purified up HiPrep column with water after 4 hour to isolate the product. LC-MS confirmed the molecular weight.

### Example 11

### Preparation of Compound 7

To a solution of Fmoc-Cys(S-tBu)—COOH (6, 1.0 g, 2.3 mmol) in anhydrous DCM (25 mL), NHS (4.6 mmol), EDC (4.6 mmol), and DMAP (4.6 mmol) were added at 0° C. followed by stirring at 0° C. to room temperature for 2 hours. The reaction mixture was washed with 0.1 N HCl twice. The organic layers were combined, dried over anhydrous sodium sulfate, and concentrated in vacuo to give the product. The product was used without further purification.

### Example 12

### Preparation of Compound 9

A solution of compound 7 in anhydrous acetonitrile is added to a solution of NH₂—C6-Oligo (8) in 6 mL of pH 7.8, 100 mM sodium phosphate and acetonitrile (1:1). After the completion of the reaction, the reaction mixture is purified on Source 15Q Column with A buffer (pH 7.0, 5 M urea, 100 mM KH₂PO₃, 25% CH₃CN) and B buffer (2 M KBr) and desalted on HiPrep with water to give the product. The molecular weight is confirmed by LC-MS.

### Example 13

### Preparation of Compound 9a

A solution of compound 7 in anhydrous acetonitrile was added to a solution of NH₂—C6-Oligo-FAM (8a, 100 mg) in 6 mL of PH 7.8, 100 mM sodium phosphate and acetonitrile (1:1). After the completion of the reaction, the reaction mixture was purified on Source 15Q Column with A buffer (pH 7.0, 5 M urea, 100 mM KH₂PO₃, 25% CH₃CN) and B buffer (2 M KBr) and desalted on HiPrep with water to give 120 mg (oligo eq.) of the product. The molecular weight was confirmed by LC-MS.

### Example 14

### Preparation of Compound 10

A solution of compound 9 in 2 mL of water is treated with 1 mL of piperidine and DMF (1:1). The reaction is stirred for 30 minutes and then desalted on HiPrep column with water to give the product. The molecular weight is confirmed by LC-MS.

### Example 15

### Preparation of Compound 10a

A solution of compound 9a (120 mg) in 2 mL of water was treated with 1 mL of piperidine and DMF (1:1). The reaction was stirred for 30 minutes and then desalted on HiPrep column with water to give 108 mg (oligo eq.) of the product. The molecular weight was confirmed by LC-MS.

### Example 16

### Preparation of Compound 12

A solution of compound 10 in 5 mL of pH 7.8 sodium phosphate (100 mM) and 2.5 mL of acetonitrile is treated with the solution of compound 11 (350 mg, 1.14 mmol) in 2.5 mL of CH₃CN. The reaction mixture is stirred for about 1 hour and desalted with water on HiPrep column to give the product. The molecular weight is confirmed by LC-MS.

### Example 17

### Preparation of Compound 12a

A solution of compound 10a (108 mg) in 5 mL of pH 7.8 sodium phosphate (100 mM) and 2.5 of acetonitrile was treated with the solution of compound 11 (350 mg, 1.14 mmol) in 2.5 mL of CH₃CN. The reaction mixture was stirred for about 1 hour and desalted with water on HiPrep column to give 104 mg (oligo eq.) of the product. The molecular weight was confirmed by LC-MS.

### Example 18

### Preparation of Compound 14

A solution of Compound 12 in 20 mL of pH 7.0, 5 M urea and 100 mM KH₂PO₄ is treated with CGVKRKKKP (compound 13, 15 mg, 4 eq.). As the reaction is completed, the mixture is purified on Source 15Q column with A buffer (pH 7.0, 5 M urea; 100 mM KH₂PO₃, 25% CH₃CN) and B buffer (2 M KBr) to give the product in urea buffer. The molecular weight is confirmed by LC-MS. The product solution is used as it is without further isolation.

### Example 19

### Preparation of Compound 14a

A solution of Compound 12a (23.9 mg, 0.037 mmol) in 10 mL of pH 7.0, 5 M urea and 100 mM KH₂PO₄ was treated with CGVKRKKKP (compound 13, 15 mg, 4 eq.). The reaction was completed in 1 hour and was purified on Source 15Q column with A buffer (pH 7.0, 5 M urea, 100 mM KH₂PO₃, 25% CH₃CN) and B buffer (2 M KBr) to give 19 mg (oligo eq.) of the product in 12 mL of urea buffer. Molecular weight was confirmed by LC-MS. The product solution was used without further isolation.

### Example 20

### Preparation of Compound 15

A solution of compound 14 is treated 5 mL of DTT (92 mg) in 100 mL of ammonium carbonate. As the reaction is completed, the mixture is desalted with 1 M urea in pH 6.5 sodium phosphate buffer to give the product in the desalting buffer. The molecular weight is confirmed by LC-MS.

### Example 21

### Preparation of Compound 15a

The solution of compound 14a was treated 5 mL of DTT (92 mg) in 100 mL of ammonium carbonate for 3 hours. The reaction was desalted with 1 M urea in pH 6.5 sodium phosphate buffer to give 27 mg (oligo eq.) of the product in 45 mL of desalting buffer. The molecular weight was confirmed by LC-MS.

### Example 22

### Preparation of Compound 16

To a solution of Compound 15 (9 mg of oligo eq.) in the desalting buffer, compound 3 or 3a is added. After the reaction completed, the mixture is purified on Source 15Q column with A buffer (pH 7.0, 5 M urea, 100 mM KH₂PO₃, 25% CH₃CN) and B buffer (2 M KBr) and desalted with PBS on HiPrep column to give the product. Molecular weight is confirmed by LC-MS.

### Example 23

### Preparation of Compound 16a

To a solution of Compound 15a (2 mg of oligo eq.) in the desalting buffer, compound 3 (1.2 mg, 4 eq.) was added. After the reaction was completed, the mixture was purified on Source 15Q column with A buffer (pH 7.0, 5 M urea, 100 mM KH₂PO₃, 25% CH₃CN) and B buffer (2 M KBr) and desalted with PBS on HiPrep column to give the product. The molecular weight was confirmed by LC-MS.

### Example 24

### Effects on Cellular Uptake and Cytoplasmic Localization of Nucleic Acids

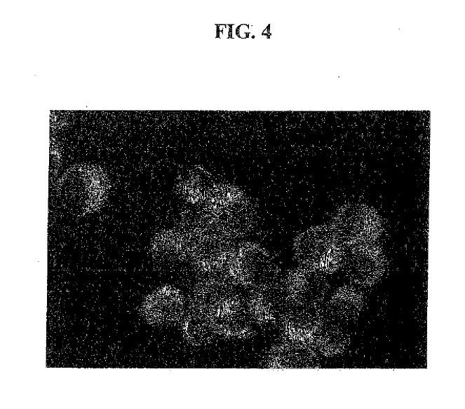

Effects of compounds described herein on cellular uptake and cytoplasmic localization of nucleic acids were evaluated in KB cells (human adenocarcinoma). The cells were maintained in complete medium (DMEM, supplemented with 10% FBS) at 37° C. The cells were treated with a solution of compound 5a (HS—C6-Oligo2-FAM: antisense ErbB3 oligonucleotide). The cells were washed with PBS, stained, and fixed with pre-cooled 70% EtOH. The samples were inspected under fluorescent microscope. A fluorescent image of the treated cell samples is shown in FIG. 4. In the image, oligonucleotides labeled with FAM are shown in the cytosol of the treated cells. The oligonucleotides were released from endosomes and diffused into the cytoplasm. The results show that the endosomal release-promoting moiety is an effective means for delivering therapeutic nucleic acids into cells and localizing them in cellular compartments, cytoplasmic area within cells.

### Example 25

### Effects on Modulation of Target Gene Expression In Vitro

Effects of the compounds described herein on modulating target gene expression are evaluated in a number of different cancer cells including epidermoid carcinoma (A431), prostate cancer (15PC3, LNCaP, PC3, CWR22), lung cancer (A549, HCC827, H1581), breast cancer (SKBR3), colon cancer (SW480), pancreatic cancer cells (BxPC3), gastric cancer cells (N87), and melanoma (518A2). Cells are treated with compound 5 (with Oligo 2 or a scrambled sequence, Oligo-3). After treatment, the intracellular mRNA levels of the target gene, such as human ErbB3, and a housekeeping gene, such as GAPDH are quantitated by RT-qPCR. The expression levels of mRNA normalized to that of GAPDH are compared. To confirm the mRNA down-regulation data, the protein level from the cells are also analyzed using conjugates of both Oligo-2 and Oligo-3 by Western Blot method.

### Example 27

### Effects on Target Gene Downregulation In Vivo

Effects of the compounds described herein on downregulating target gene expression are evaluated in mice xenografted with human cancer cells. Xenograft tumors are established in mice by injecting human cancer cells. 15PC3 human prostate tumors are established in nude mice by subcutaneous injection of 5×10⁶ cells/mouse into the right auxiliary flank. When tumors reach approximately 100 mm³, the mice are treated with compound 5 (Oligo 2) intravenously (i.v.) (alternatively, intraperitoneally) or at 60 mg/kg, 45 mg/kg, 30 mg/kg, 25 mg/kg, 15 mg/kg, or 5 mg/kg/dose (equivalent of Oligo2) at q3d×4 or more. The dosage is based on the amounts of oligonucleotides contained in compound 5. The mice are sacrificed twenty four hours after the final dose. Plasma samples are collected from the mice and stored at −20° C. Tumor and liver samples are also collected from the mice. The samples were analyzed for mRNA KD.

1. A compound of Formula (I): 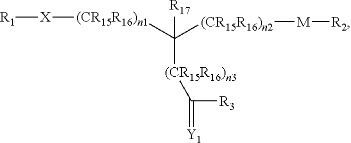
wherein
R₁ is a group of Formula (Ia₁) or (Ia₂): 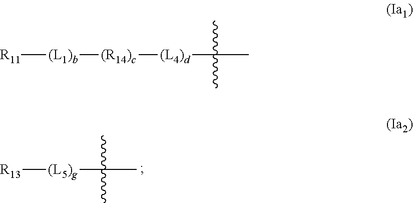
X is O or S;
R₂ is hydrogen, a leaving group, a functional group, a targeting group, a non-antigenic polymer, or a group of Formula (Ib₁), (Ib₂), or (Ib₃): 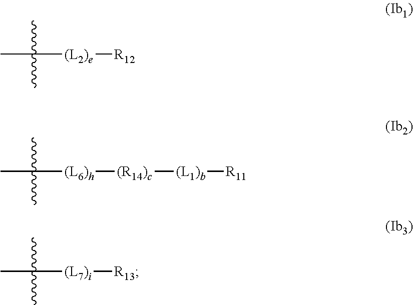
M is O, or NR₅;
R₃ is OH, OR₆, SH, SR₇, a leaving group, a functional group, a targeting group, a non-antigenic polymer or a group of Formula (Ic₁), (Ic₂) or (Ic₃): 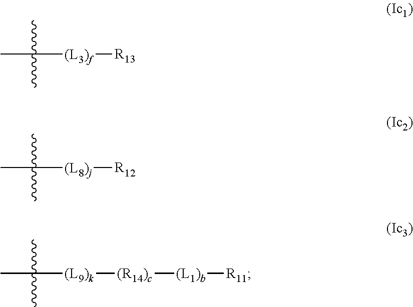
Y₁ is O, S, or NR₈;
R₄ is C₁₋₆ alkyl, C₁₋₆ branched alkyl or 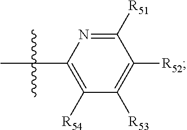
wherein R₅₁₋₅₄ are independently selected from a group consisting of hydrogen, amino, azido, carboxy, cyano, halo, hydroxyl, nitro, hydrogen, C₁₋₆ alkyl, C₃₋₈ branched alkyl, C₃₋₈ cycloalkyl, C₁₋₆ substituted alkyl, C₃₋₈ substituted cycloalkyl, aryl and substituted aryl;
R₅ and R₈ are independently selected from the group consisting of hydrogen, amino, azido, carboxy, cyano, halo, hydroxyl, nitro, hydrogen, C₁₋₆ alkyl, C₃₋₈ branched alkyl, C₃₋₈ cycloalkyl, C₁₋₆ substituted alkyl, C₃₋₈ substituted cycloalkyl, aryl and substituted aryl;
R₆ and R₇ are independently C₁₋₆ alkyl, or C₁₋₆ branched alkyl,
R₁₁ is hydrogen, C₁₋₆ alkyl, a functional group, a targeting group, or an endosomal release-promoting moiety;
R₁₂ is hydrogen, C₁₋₆ alkyl, a leaving group, a functional group, a targeting group, a nuclear localization signal peptide, or a non-antigenic polymer;
R₁₃ is selected from the group consisting of OH, OR₆, SH, SR₇, a leaving group, a functional group, a targeting group, a biologically active agent, and a non-antigenic polymer, or 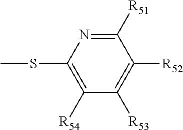
wherein a group of Formula (Ia₂) is present and (g) is zero;
R₁₄ is an endosomal release-promoting moiety;
R₁₅₋₁₇ are independently selected from the group consisting of hydrogen, C₁₋₆ alkyls, C₂₋₆ alkenyl, C₂₋₆ alkynyl, C₃₋₁₉ branched alkyl, C₃₋₈ cycloalkyl, and C₁₋₆ alkoxy, wherein R₁₅₋₁₇ in each occurrence are independently the same or different;
L₁₋₃ and L₆₋₉ are independently selected bifunctional linkers, wherein L₁₋₃ and L₆₋₉ in each occurrence are independently the same or different;
L₄₋₅ are independently selected bifunctional spacers containing a terminal sulfur adjacent to X;
(c) is zero or 1;
(d) and (g) are independently zero or 1;
(b), (e), (f), (h), (i), (j) and (k) are independently zero or positive integers; and
(n1) is zero a positive integer of from about 1 to about 10;
(n2) and (n3) are independently zero or positive integers of from about 1 to about 10, provided that at least one of R₁₋₃ includes an endosomal release-promoting moiety, and provided that at least one of the remaining R₁₋₃ includes a biologically active agent, or 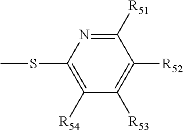
when a group of Formula (Ia₂) is present and (g) is zero.

2.-4. (canceled)

5. The compound of claim 1, wherein the compound has Formula (III):

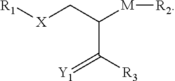

6. (canceled)

7. The compound of claim 5 having Formula (IIIa) or (III′a): 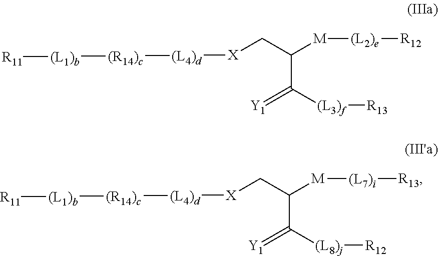
wherein at least one of R₁₁ and R₁₄ includes an endosomal release-promoting moiety and R₁₃ includes a biologically active agent.

8. (canceled)

9. The compound of claim 7, having Formula (IVa) or (IV′a): 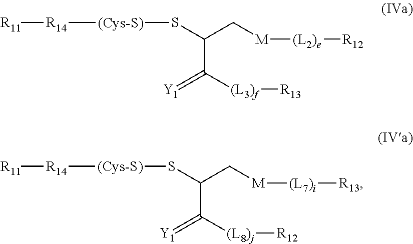
wherein
R₁₁ is hydrogen, a targeting group or a histidine-rich peptide;
R₁₂ is hydrogen, C₁₋₆ alkyl, a leaving group, a functional group, or a nuclear localization signal peptide;
R₁₃ includes a biologically active agent; and
R₁₄ is a histidine-rich peptide.

10. (canceled)

11. The compound of claim 7, having Formula (Va) or (V′a): 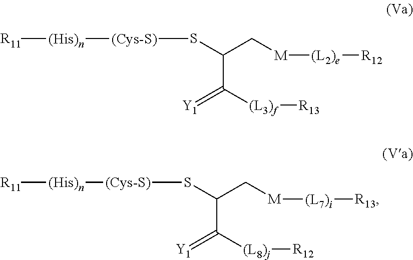
wherein
R₁₁ is hydrogen, a targeting group or a histidine-rich peptide;
R₁₂ is hydrogen, C₁₋₆ alkyl, a leaving group, a functional group, a nuclear localization signal peptide or a non-antigenic polymer;
R₁₃ includes a biologically active agent;
His is histidine; and
(n) is a positive integer equal to or greater than 3.

12. The compound of claim 5 having Formula (IIIb) or (III′b): 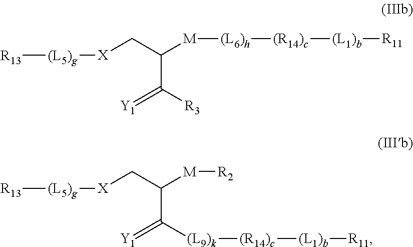
wherein
at least one of R₁₁ and R₁₄ includes an endosomal release-promoting moiety;
R₁₃ is a biologically active agent when (g) is zero or 1, or 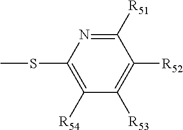
wherein (g) is zero;
R₂ is hydrogen, a leaving group, a functional group, a targeting group, a non-antigenic polymer; and
R₃ is OH, OR₆, a leaving group, a functional group, a targeting group, a non-antigenic polymer.

13. The compound of claim 12, having Formula (IVb) or (IV′b): 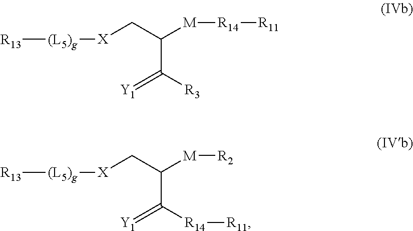
wherein
R₁₁ is hydrogen or a targeting group;
R₁₃ is a biologically active agent when (g) is zero or 1, or 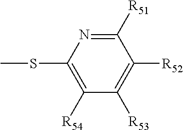
wherein (g) is zero;
R₁₄ is a histidine-rich peptide;
R₂ is hydrogen, a leaving group, a functional group, a targeting group, a non-antigenic polymer; and
R₃ is OH, OR₆, a leaving group, a functional group, a targeting group, a non-antigenic polymer.

14. (canceled)

15. The compound of claim 12, having Formula (Vb) or (V′b): 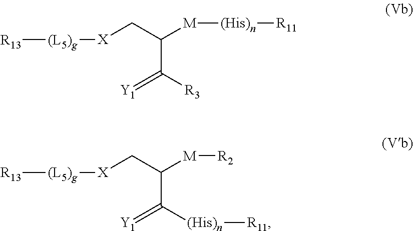
wherein
R₁₁ is hydrogen or a targeting group;
R₁₃ is a biologically active agent when (g) is zero or 1, or 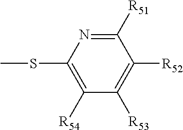
when (g) is zero;
R₂ is hydrogen, a leaving group, a functional group, a targeting group, a non-antigenic polymer;
R₃ is OH, OR₆, a leaving group, a functional group, a targeting group, a non-antigenic polymer;
His is histidine; and
(n) is a positive integer equal to or greater than 3.

16.-17. (canceled)

18. The compound of claim 1, wherein the endosomal release-promoting moiety includes a histidine-rich peptide, containing about 3 to 25 amino acids, and the histidine-rich peptide contains histidines ranging from about 30% to about 100%.

19.-20. (canceled)

21. The compound of claim 1, wherein the nuclear localization signal peptide is selected from the group consisting of CGVKRKKKP (SEQ ID NO: 28), CYGRKKRRQRRR (SEQ ID NO: 29), YGRKKRRQRRRC (SEQ ID NO: 30), YGRKKRRQRRR (SEQ ID NO: 31), PKKKRKVEDPYC (SEQ ID NO: 32), VQRKRQKLM (SEQ ID NO:33), and CGYGPKKKRKVGG (SEQ ID NO: 34).

22. The compound of claim 1, wherein L₁₋₃ and L₆₋₉ are independently selected from the group consisting of
—(CR₂₁R₂₂)ₜ₁—[C(═Y₁₆)]ₐ₃—,
—(CR₂₁R₂₂)ₜ₁Y₁₇—(CR₂₃R₂₄)ₜ₂—(Y₁₈)ₐ₂—[C(═Y₁₆)]ₐ₃—,
—(CR₂₁R₂₂CR₂₃R₂₄Y₁₇)ₜ₁—[C(═Y₁₆)]ₐ₃—,
—(CR₂₁R₂₂CR₂₃R₂₄Y₁₇)ₜ₁ (CR₂₅R₂₆)ₜ₄—(Y₁₈)ₐ₂—[C(═Y₁₆)]ₐ₃—,
—[(CR₂₁R₂₂CR₂₃R₂₄)ₜ₂Y₁₇]ₜ₃(CR₂₅R₂₆)ₜ₄—(Y₁₈)ₐ₂—[C(═Y₁₆)]ₐ₃—,
—(CR₂₁R₂₂)ₜ₁—[(CR₂₃R₂₄)ₜ₂Y₁₇]ₜ₃(CR₂₅R₂₆)ₜ₄—(Y₁₈)ₐ₂—[C(═Y₁₆)]ₐ₃—,
—(CR₂₁R₂₂)ₜ₁ (Y₁₇)ₐ₂[C(═Y₁₆)]ₐ₃(CR₂₃R₂₄)ₜ₂—,
—(CR₂₁R₂₂)ₜ₁ (Y₁₇)ₐ₂[C(═Y₁₆)]ₐ₃Y₁₄(CR₂₃R₂₄)ₜ₂—,
—(CR₂₁R₂₂)ₜ₁(Y₁₇)ₐ₂[C(═Y₁₆)]ₐ₃(CR₂₃R₂₄)ₜ₂—Y₁₅—(CR₂₃R₂₄)ₜ₃—,
—(CR₂₁R₂₂)ₜ₁(Y₁₇)ₐ₂[C(═Y₁₆)]ₐ₃Y₁₄(CR₂₃R₂₄)ₜ₂—Y₁₅—(CR₂₃R₂₄)ₜ₃—,
—(CR₂₁R₂₂)ₜ₁(Y₁₇)ₐ₂[C(═Y₁₆)]ₐ₃(CR₂₃R₂₄CR₂₅R₂₆Y₁₉)ₜ₂(CR₂₇CR₂₈)ₜ₃—,
—(CR₂₁R₂₂)ₜ₁(Y₁₇)ₐ₂[C(═Y₁₆)]ₐ₃Y₁₄(CR₂₃R₂₄CR₂₅R₂₆Y₁₉)ₜ₂(CR₂₇CR₂₈)ₜ₃—, 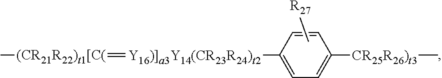
—(CH₂)₄—C(═O)—, —(CH₂)₅—C(═O)—, —(CH₂)₆—C(═O)—,
—CH₂CH₂O—CH₂O—C(═O)—, —(CH₂CH₂O)₂—CH₂O—C(═O)—,
—(CH₂CH₂O)₃—CH₂O—C(═O)—, —(CH₂CH₂O)₂—C(═O)—,
—CH₂CH₂O—CH₂CH₂NH—C(═O)—,
—(CH₂CH₂O)₂—CH₂CH₂NH—C(═O)—,
—CH₂—O—CH₂CH₂O—CH₂CH₂NH—C(═O)—,
—CH₂—O—(CH₂CH₂O)₂—CH₂CH₂NH—C(═O)—,
—CH₂—O—CH₂CH₂O—CH₂C(═O)—,
—CH₂—O—(CH₂CH₂O)₂—CH₂C(═O)—,
—(CH₂)₄—C(═O)NH—, —(CH₂)₅—C(═O)NH—, —(CH₂)₆—C(═O)NH—,
—CH₂CH₂O—CH₂O—C(═O)—NH—,
—(CH₂CH₂O)₂—CH₂O—C(═O)—NH—,
—(CH₂CH₂O)₃—CH₂O—C(═O)—NH—,
—(CH₂CH₂O)₂—C(═O)—NH—,
—CH₂CH₂O—CH₂CH₂NH—C(═O)—NH—,
—(CH₂CH₂O)₂—CH₂CH₂NH—C(═O)—NH—,
—CH₂—O—CH₂CH₂O—CH₂CH₂NH—C(═O)—NH—,
—CH₂—O—(CH₂CH₂O)₂—CH₂CH₂NH—C(═O)—NH—,
—CH₂—O—CH₂CH₂O—CH₂C(═O)—NH—,
—CH₂—O—(CH₂CH₂O)₂—CH₂C(═O)—NH—,
—(CH₂CH₂O)₂—, —CH₂CH₂O—CH₂O—,
—(CH₂CH₂O)₂—CH₂CH₂NH—, —(CH₂CH₂O)₃—CH₂CH₂NH—,
—CH₂CH₂O—CH₂CH₂NH—, —(CH₂CH₂O)₂—CH₂CH₂NH—,
—CH₂—O—CH₂CH₂O—CH₂CH₂NH—,
—CH₂—O—(CH₂CH₂O)₂—CH₂CH₂NH—,
—CH₂—O—CH₂CH₂O—, —CH₂—O—(CH₂CH₂O)₂—,
—(CH₂)₄—, —(CH₂)₃—, —O(CH₂)₂—, —C(═O)O(CH₂)₃—, —C(═O)NH(CH₂)₃—,
—C(═O)(CH₂)₂—, —C(═O)(CH₂)₃—,
—CH₂—C(═O)—O(CH₂)₃—, —CH₂—C(═O)—NH(CH₂)₃—,
—CH₂—OC(═O)—O(CH₂)₃—, —CH₂—OC(═O)—NH(CH₂)₃—,
—(CH₂)₂—C(═O)—O(CH₂)₃—, —(CH₂)₂—C(═O)—NH(CH₂)₃—,
—CH₂C(═O)O(CH₂)₂—O—(CH₂)₂—,
—CH₂C(═O)NH(CH₂)₂—O—(CH₂)₂—,
—(CH₂)₂C(═O)O(CH₂)₂—O—(CH₂)₂—,
—(CH₂)₂C(═O)NH(CH₂)₂—O—(CH₂)₂—,
—CH₂C(═O)O(CH₂CH₂O)₂CH₂CH₂—,
—(CH₂)₂C(═O)O(CH₂CH₂O)₂CH₂CH₂—, 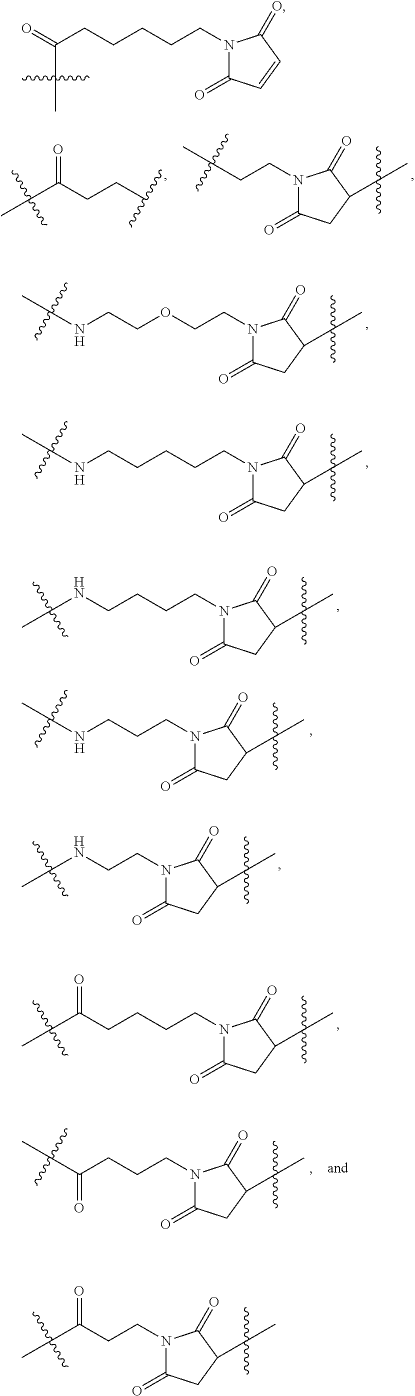
wherein:
Y₁₆ is O, NR₂₈, or S;
Y₁₄₋₁₅ and Y₁₇₋₁₉ are independently O, NR₂₉, or S;
R₂₁₋₂₇ are independently selected from the group consisting of hydrogen, hydroxyl, carboxyl, amine, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;
R₂₈₋₂₉ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;
(t1), (t2), (t3) and (t4) are independently zero or positive integers;
(a2) and (a3) are independently zero or 1;
wherein L₂ and L₆₋₇ in each occurrence are independently the same or different when (e), (h) or (i) is equal to or greater than 2; and
wherein L₃ and L₈₋₉ in each occurrence are independently the same or different when (f), (j) or (j) is equal to or greater than 2.

23.-25. (canceled)

26. The compound of claim 1, wherein L₄₋₅ are independently selected from the group consisting of:
—(CR′₂₁R′₂₂)_{t′1}—[(C(═Y′₁₆)]_{a′3}(CR′₂₇CR′₂₈)_{t′2}S—,
—(CR′₂₁R′₂₂)_{t′1}Y′₁₄—(CR′₂₃R′₂₄)_{t′2}—(Y′₁₅)_{1′2}—[C(═Y′₁₆)]_{a′3}(CR′₂₇CR′₂₈)_{t′3}S—,
—(CR′₂₁R′₂₂CR′₂₃R′₂₄Y′₁₄)_{t′1}—[C(═Y′₁₆)]_{a′3}(CR′₂₇CR′₂₈)_{t′2}S—,
—(CR′₂₁R′₂₂CR′₂₃R′₂₄Y′₁₄)_{t′1}(CR′₂₅R′₂₆)_{t′2}—(Y′₁₅)_{a′2}—[C(═Y′₁₆)]_{a′3}(CR′₂₇CR′₂₈)_{t′3}S—,
—[(CR′₂₁R′₂₂CR′₂₃R′₂₄)_{t′2}Y′₁₄]_{t′1}(CR′₂₅R′₂₆)_{t′2}—(Y′₁₅)_{a′2}—[C(═Y′₁₆)]_{a′3}(CR′₂₇CR′₂₈)_{t′3}S—,
—(CR′₂₁R′₂₂)_{t′1}—[(CR′₂₃R′₂₄)_{t′2}Y′₁₄]_{t′2}(CR′₂₅R′₂₆)_{t′3}—(Y′₁₅)_{a′2}—[C(═Y′₁₆)]_{a′3}(CR′₂₇CR′₂₈)_{t′4}S—,
—(CR′₂₁R′₂₂)_{t′1}(Y′₁₄)_{a′2}[C(═Y′₁₆)]_{a′3}(CR′₂₃R′₂₄)_{t′2}S—,
—(CR′₂₁R′₂₂)_{t′1}(Y′₁₄)_{a′2}[C(═Y′₁₆)]_{a′3}Y′₁₅(CR′₂₃R′₂₄)_{t′2}S—,
—(CR′₂₁R′₂₂)_{t′1}(Y′₁₄)_{a′2}[C(═Y′₁₆)]_{a′3}(CR′₂₃R′₂₄)_{t′2}—Y′₁₅—(CR′₂₃R′₂₄)_{t′3}S—,
—(CR′₂₁R′₂₂)_{t′1}(Y′₁₄)_{a′2}[C(═Y′₁₆)]_{a′3}Y′₁₄(CR′₂₃R′₂₄)_{t′2}—Y′₁₅—(CR′₂₃R′₂₄)_{t′3}S—,
—(CR′₂₁R′₂₂)_{t′1}(Y′₁₄)_{a′2}[C(═Y′₁₆)]_{a′3}(CR′₂₃R′₂₄CR′₂₅R′₂₆Y′₁₅)_{t′2}(CR′₂₇CR′₂₈)_{t′3}S—,
—(CR′₂₁R′₂₂)_{t′1}(Y′₁₄)_{a′2}[C(═Y′₁₆)]_{a′3}Y′₁₇(CR′₂₃R′₂₄CR′₂₅R′₂₆Y′₁₅)_{t′2}(CR′₂₇CR′₂₈)_{t′3}S—, 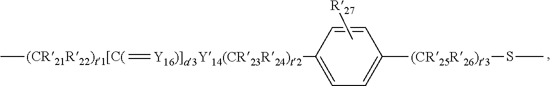
—(CH)₆—S—, —(CH)₅—S—, —(CH)₄—S—, —(CH)₃—S—, —(CH)₂—S—,
—(CH₂)₄—C(═O)NH—CH(COOH)CH₂S—,
—(CH₂)₅—C(═O)NH—CH(COOH)CH₂S—,
—(CH₂)₆—C(═O)NH—CH(COOH)CH₂S—,
—CH₂CH₂O—CH₂O—C(═O)NH—CH(COOH)CH₂S—,
—(CH₂CH₂O)₂—CH₂O—C(═O)NH—CH(COOH)CH₂S—,
—(CH₂CH₂O)₃—CH₂O—C(═O)NH—CH(COOH)CH₂S—,
—(CH₂CH₂O)₂—C(═O)NH—CH(COOH)CH₂S—,
—CH₂CH₂O—CH₂CH₂NH—C(═O)NH—CH(COOH)CH₂S—,
—(CH₂CH₂O)₂—CH₂CH₂NH—C(═O)NH—CH(COOH)CH₂S—,
—CH₂—O—CH₂CH₂O—CH₂CH₂NH—C(═O)NH—CH(COOH)CH₂S—,
—CH₂—O—(CH₂CH₂O)₂—CH₂CH₂NH—C(═O)NH—CH(COOH)CH₂S—,
—CH₂—O—CH₂CH₂O—CH₂C(═O)NH—CH(COOH)CH₂S—,
—CH₂—O—(CH₂CH₂O)₂—CH₂C(═O)NH—CH(COOH)CH₂S—,
—(CH₂)₄—C(═O)NHCH(COOH)CH₂S—,
—(CH₂CH₂O)₂CH₂C(═O)NH—CH(COOH)CH₂S—,
—CH₂CH₂O—CH₂OC(═O)NH—CH(COOH)CH₂S—,
—(CH₂CH₂O)₂—CH₂CH₂NHC(═O)CH(NH₂)CH₂S—,
—(CH₂CH₂O)₃—CH₂CH₂NHC(═O)CH(NH₂)CH₂S—,
—CH₂CH₂O—CH₂CH₂NHC(═O)CH(NH₂)CH₂S—,
—(CH₂CH₂O)₂—CH₂CH₂NHC(═O)CH(NH₂)CH₂S—,
—CH₂—O—CH₂CH₂O—CH₂CH₂NHC(═O)CH(NH₂)CH₂S—,
—CH₂—O—(CH₂CH₂O)₂—CH₂CH₂NHC(═O)CH(NH₂)CH₂S—,
—CH₂—O—CH₂CH₂O—CH₂C(═O)NHCH(COOH)CH₂S—, and
—CH₂—O—(CH₂CH₂O)₂—CH₂C(═O)NHCH(COOH)CH₂S—
wherein:
Y′₁₆ is O, NR′₂₈, or S;
Y′₁₄₋₁₅ and Y′₁₇ are independently O, NR′₂₉, or S;
R′₂₁₋₂₇ are independently selected from the group consisting of hydrogen, hydroxyl, carboxyl, amine, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;
R′₂₈₋₂₉ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;
(t′1), (t′2), (t′3) and (t′4) are independently zero or positive integers; and
each (a′2) and (a′3) are independently zero or 1.

27.-31. (canceled)

32. The compound of claim 1, wherein the biologically active agent is selected from the group consisting of —NH₂ containing moieties, —OH containing moieties and —SH containing moieties.

33. The compound of claim 32, wherein the biological active agent is an oligonucleotide.

34.-35. (canceled)

36. The compound of claim 33, wherein the oligonucleotide is selected from the group consisting of deoxynucleotide, ribonucleotide, locked nucleic acids (LNA), short interfering RNA (siRNA), microRNA (miRNA), aptamers, peptide nucleic acid (PNA), phosphorodiamidate morpholino oligonucleotides (PMO), tricyclo-DNA, double stranded oligonucleotide (decoy ODN), catalytic RNA (RNAi), aptamers, spiegelmers, CpG oligomers and combinations thereof.

37.-39. (canceled)

40. The compound of claim 33, wherein the oligonucleotide modulates expression of oncogenes, pro-angiogenesis pathway genes, pro-cell proliferation pathway genes, viral infectious agent genes, and pro-inflammatory pathway genes.

41. The compound of claim 33, wherein the oligonucleotide is selected from the group consisting of antisense bcl-2 oligonucleotides, antisense HIF-1α oligonucleotides, antisense survivin oligonucleotides, antisense ErbB3 oligonucleotides, antisense PIK3CA oligonucleotides, antisense HSP27 oligonucleotides, antisense androgen receptor oligonucleotides, antisense Gli2 oligonucleotides, and antisense beta-catenin oligonucleotides.

42. The compound of claim 33, wherein the oligonucleotide comprises eight or more consecutive nucleotides set forth in SEQ ID NO: 1, SEQ ID NOs 2 and 3, SEQ ID NO:4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, and SEQ ID NO: 16, and each nucleic acid is a naturally occurring or modified nucleic acid.

43. The compound of claim 33, wherein the targeting group is selected from the group consisting of RGD peptides, folate, anisamide, vascular endothelial cell growth factor, FGF2, somatostatin and somatostatin analogs, transferrin, melanotropin, ApoE and ApoE peptides, von Willebrand's Factor and von Willebrand's Factor peptides, adenoviral fiber protein and adenoviral fiber protein peptides, PD1 and PD1 peptides, EGF and EGF peptides.

44. The compound of claim 1 selected from the group consisting of: 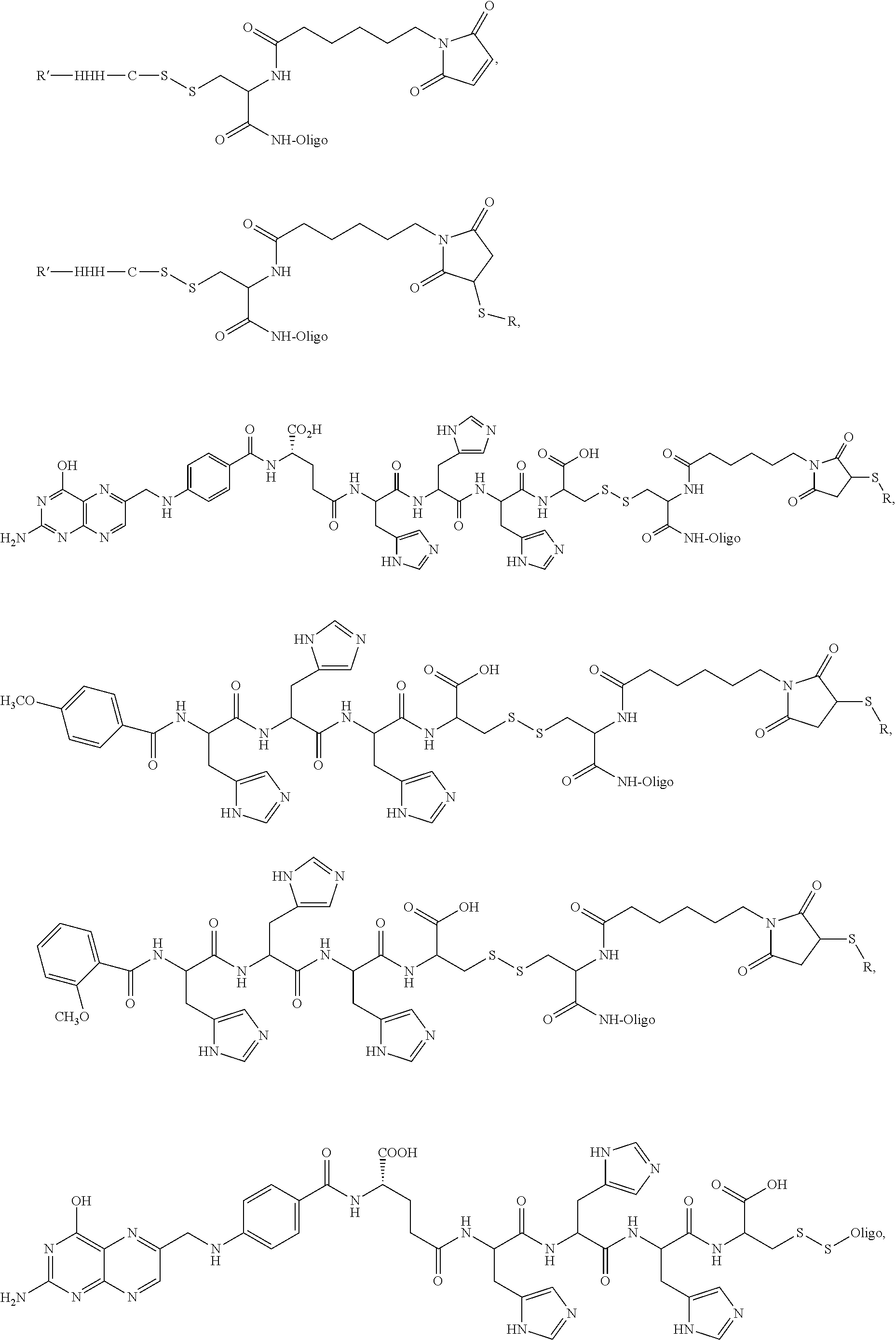 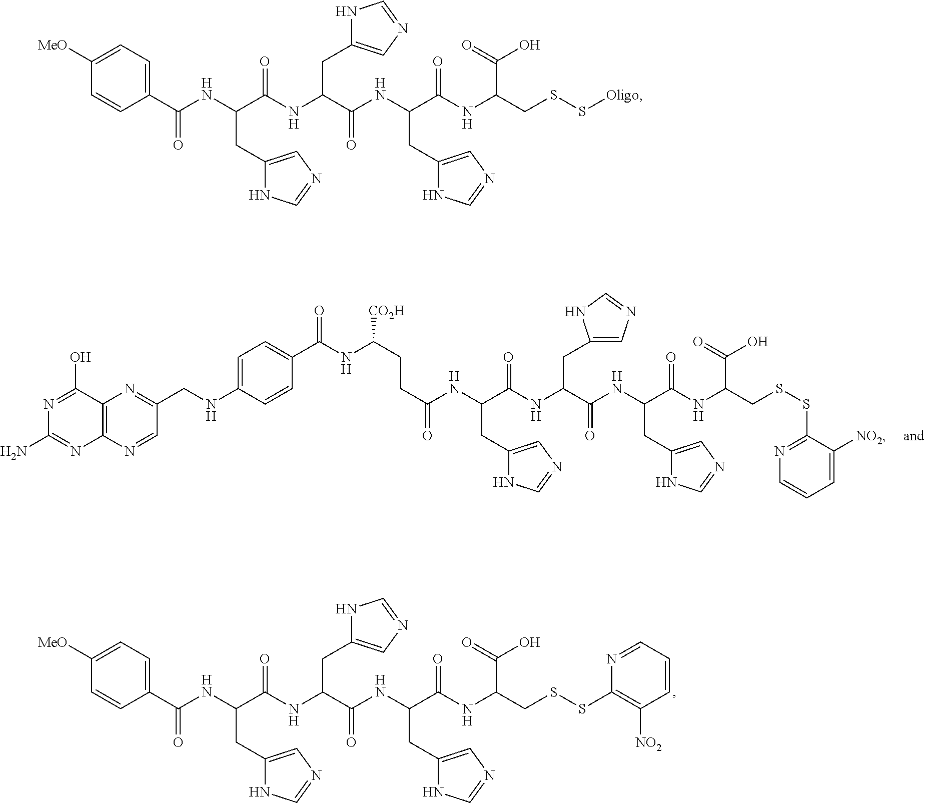
wherein
Oligo is an oligonucleotide;
R′ is a targeting group; and
R is a nuclear localization signal peptide.

45. A nanoparticle composition comprising the compound of claim 1.

46.-48. (canceled)

49. A method of inhibiting or downregulating a gene expression in human cells or tissues, comprising:
contacting human cells or tissues with a compound of claim 1,
wherein at least one of R₁₋₃ includes an endosomal release-promoting moiety, and at least one of the remaining R₁₋₃ includes an oligonucleotide.

50. The method of claim 49, wherein the cells or tissues are cancer cells or tissues.

51. (canceled)

52. A method of inhibiting the growth or proliferation of cancer cells comprising:
contacting a cancer cell with the compound of claim 1,
wherein at least one of R₁₋₃ includes an endosomal release-promoting moiety, and at least one of the remaining R₁₋₃ includes an oligonucleotide.

53. The method of claim 52, further comprising administering an anticancer agent.

54. A method of treating a disease in a mammal comprising
administering an effective amount of a compound of claim 1 to a mammal in need thereof, or a nanoparticle composition of comprising the compound of claim 1,
wherein at least one of R₁₋₃ includes an endosomal release-promoting moiety; and at least one of the remaining R₁₋₃ includes an oligonucleotide.


SEQUENCE LISTING (35 sequences)

SEQ ID NO: 1 - DNA, 16 bp, Artificial Sequence
  Description of Artifical Sequence Synthetic oligonucleotide
ctcaatccatggcagc

SEQ ID NO: 2 - DNA, 21 bp, Artificial Sequence
  Description of Combined DNA/RNA Molecule Synthetic oligonucleotide
gcaugcggccucuguuugatt

SEQ ID NO: 3 - DNA, 21 bp, Artificial Sequence
  Description of Combined DNA/RNA Molecule Synthetic oligonucleotide
ucaaacagaggccgcaugctt

SEQ ID NO: 4 - DNA, 19 bp, Artificial Sequence
  Description of Artifical Sequence Synthetic oligonucleotide
tctcccagcgtgcgcccat

SEQ ID NO: 5 - DNA, 16 bp, Artificial Sequence
  Description of Artifical Sequence Synthetic oligonucleotide
tggcaagcatcctgta

SEQ ID NO: 6 - DNA, 16 bp, Artificial Sequence
  Description of Artifical Sequence Synthetic oligonucleotide
tagcctgtcacttctc

SEQ ID NO: 7 - DNA, 16 bp, Artificial Sequence
  Description of Artifical Sequence Synthetic oligonucleotide
gctccagacatcactc

SEQ ID NO: 8 - DNA, 16 bp, Artificial Sequence
  Description of Artifical Sequence Synthetic oligonucleotide
agccattcattccacc

SEQ ID NO: 9 - DNA, 16 bp, Artificial Sequence
  Description of Artifical Sequence Synthetic oligonucleotide
ttattgtgcatctcag

SEQ ID NO: 10 - DNA, 16 bp, Artificial Sequence
  Description of Artifical Sequence Synthetic oligonucleotide
cgtgtatttccgcgtg

SEQ ID NO: 11 - DNA, 16 bp, Artificial Sequence
  Description of Artifical Sequence Synthetic oligonucleotide
ggcacagccagtggcg

SEQ ID NO: 12 - DNA, 16 bp, Artificial Sequence
  Description of Artifical Sequence Synthetic oligonucleotide
cccaaggcactgcaga

SEQ ID NO: 13 - DNA, 16 bp, Artificial Sequence
  Description of Artifical Sequence Synthetic oligonucleotide
accaagtttcttcagc

SEQ ID NO: 14 - DNA, 16 bp, Artificial Sequence
  Description of Artifical Sequence Synthetic oligonucleotide
ctccttggtgcagtct

SEQ ID NO: 15 - DNA, 15 bp, Artificial Sequence
  Description of Artifical Sequence Synthetic oligonucleotide
tcagattcaaaccca

SEQ ID NO: 16 - DNA, 16 bp, Artificial sequence
  Description of Artifical Sequence Synthetic oligonucleotide
gtgttctacaccatta

SEQ ID NO: 17 - protein, 12 aa, Artificial sequence
  Description of Artifical Sequence Synthetic peptide
CYGRKKRRQRRR

SEQ ID NO: 18 - protein, 10 aa, Artificial sequence
  Description of Artifical Sequence Synthetic peptide
CRRRRRRRRR

SEQ ID NO: 19 - protein, 23 aa, Artificial sequence
  Description of Artifical Sequence Synthetic peptide
CYGRKKRRQRRRYGRKKRRQRRR

SEQ ID NO: 20 - protein, 4 aa, Artificial sequence
  Description of Artifical Sequence Synthetic peptide
RGDC

SEQ ID NO: 21 - protein, 5 aa, Artificial sequence
  Description of Artifical Sequence Synthetic peptide
RGDFC

SEQ ID NO: 22 - protein, 5 aa, Artificial sequence
  Description of Artifical Sequence Synthetic peptide
RGDFK

SEQ ID NO: 23 - protein, 19 aa, Artificial sequence
  Description of Artifical Sequence Synthetic peptide
CYGRKKRRQRRRGGGRGDS

SEQ ID NO: 24 - protein, 9 aa, Artificial sequence
  Description of Artifical Sequence Synthetic peptide
RRRRRRRRR

SEQ ID NO: 25 - protein, 4 aa, Artificial sequence
  Description of Artifical Sequence Synthetic peptide
HHHK

SEQ ID NO: 26 - protein, 8 aa, Artificial sequence
  Description of Artifical Sequence Synthetic peptide
HHHKHHHK

SEQ ID NO: 27 - protein, 8 aa, Artificial sequence
  Description of Artifical Sequence Synthetic peptide
HHHHHHHH

SEQ ID NO: 28 - protein, 9 aa, Artificial sequence
  Description of Artifical Sequence Synthetic peptide
CGVKRKKKP

SEQ ID NO: 29 - protein, 12 aa, Artificial sequence
  Description of Artifical Sequence Synthetic peptide
CYGRKKRRQRRR

SEQ ID NO: 30 - protein, 12 aa, Artificial sequence
  Description of Artifical Sequence Synthetic peptide
YGRKKRRQRRRC

SEQ ID NO: 31 - protein, 11 aa, Artificial sequence
  Description of Artifical Sequence Synthetic peptide
YGRKKRRQRRR

SEQ ID NO: 32 - protein, 12 aa, Artificial sequence
  Description of Artifical Sequence Synthetic peptide
PKKKRKVEDPYC

SEQ ID NO: 33 - protein, 9 aa, Artificial sequence
  Description of Artifical Sequence Synthetic peptide
VQRKRQKLM

SEQ ID NO: 34 - protein, 13 aa, Artificial sequence
  Description of Artifical Sequence Synthetic peptide
CGYGPKKKRKVGG

SEQ ID NO: 35 - DNA, 16 bp, Artificial Sequence
  Description of Artifical Sequence Synthetic oligonucleotide
tagcttgtcccatctc